(19) United States
(12) Patent Application Publication
(10) Pub. No.: US 2025/0320482 A1
(43) Pub. Date: Oct. 16, 2025

(54) 3-METHYLCROTONIC ACID DECARBOXYLASE (MDC) VARIANTS

(71) Applicant: Global Bioenergies, Evry Cedex (FR)

(72) Inventors: Benoît Villiers, Tours (FR); François Stricher, Wolfisheim (FR)

(73) Assignee: Global Bioenergies, Evry Cedex (FR)

(21) Appl. No.: 18/477,601

(22) PCT Filed: Mar. 30, 2022
(86) PCT No.: PCT/EP2022/058372

§ 371 Date: Sep. 29, 2023

(30) Foreign Application Priority Data
Mar. 31, 2021 (EP) 21166368.7

(51) Int. Cl. C12N 9/88; C12P 5/02

(52) U.S. Cl. CPC C12N 9/88; C12P 5/026; C12Y 401/01035

(57) ABSTRACT

Described are 3-methylcrotonic acid decarboxylase (MDC) variants showing an improved activity in converting 3-methylcrotonic acid into isobutene as well as methods for the production of isobutene using such enzyme variants.

### CROSS-REFERENCE TO RELATED APPLICATIONS

This application is a § 371 National Stage Application of PCT/EP2022/058372 filed on Mar. 30, 2022, which claims priority to EP 21166368.7 filed on Mar. 31, 2021, both documents are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Mar. 11, 2024, is named Second_CorrectedSL_GB-36_US_ST25.txt and is 19,512 bytes in size.

Described are 3-methylcrotonic acid decarboxylase (MDC) variants showing an improved activity in converting 3-methylcrotonic acid into isobutene as well as methods for the production of isobutene using such enzyme variants.

A large number of chemical compounds are currently derived from petrochemicals. Alkenes (such as ethylene, propylene, the different butenes, or else the pentenes, for example) are used in the plastics industry, for example for producing polypropylene or polyethylene, and in other areas of the chemical industry and that of fuels.

Butylene exists in four forms, one of which, isobutene (also referred to as isobutylene), enters into the composition of methyl-tert-butyl-ether (MTBE), an anti-knock additive for automobile fuel. Isobutene can also be used to produce isooctene, which in turn can be reduced to isooctane (2,2,4-trimethylpentane); the very high octane rating of isooctane makes it the best fuel for so-called “gasoline” engines. Alkenes such as isobutene are currently produced by catalytic cracking of petroleum products (or by a derivative of the Fischer-Tropsch process in the case of hexene, from coal or gas). The production costs are therefore tightly linked to the price of oil. Moreover, catalytic cracking is sometimes associated with considerable technical difficulties which increase process complexity and production costs.

The production by a biological pathway of alkenes such as isobutene is called for in the context of a sustainable industrial operation in harmony with geochemical cycles. The first generation of biofuels consisted in the fermentative production of ethanol, as fermentation and distillation processes already existed in the food processing industry. The production of second generation biofuels is in an exploratory phase, encompassing in particular the production of long chain alcohols (butanol and pentanol), terpenes, linear alkanes and fatty acids. Two recent reviews provide a general overview of research in this field: Ladygina et al. (Process Biochemistry 41 (2006), 1001) and Wackett (Current Opinions in Chemical Biology 21 (2008), 187).

Different routes for the enzymatic generation of isobutene have previously been described; see, e.g., Fujii et al. (Appl. Environ. Microbiol. 54 (1988), 583); Gogerty et al. (Appl. Environm. Microbiol. 76 (2010), 8004-8010) and van Leeuwen et al. (Appl. Microbiol. Biotechnol. 93 (2012), 1377-1387) and WO2010/001078.

In addition to these routes, there are also alternative routes for the provision of isobutene utilizing the enzymatic conversion of 3-methylcrotonic acid into isobutene by a decarboxylation reaction. A decarboxylation is a chemical reaction that removes a carboxyl group and releases carbon dioxide (CO₂).

The decarboxylation of 3-methylcrotonic acid has already been suggested in US-A1-2009/0092975 while there is no experimental evidence for this conversion. In US-A1-2009/0092975, a nucleic acid sequence called PAD1 derived from Saccharomyces cerevisiae is described and is disclosed to encode a decarboxylation enzyme. This enzyme is suggested to be useful as a selectable marker in a recombinant organism while it is described that a “weak acid” may be used as the selecting agent. 3-methylcrotonic acid is mentioned, among many others, as a potential “weak acid”.

However, it was only later found that the above PAD1, in reality, does not provide for the decarboxylase activity.

In fact, the bacterial ubiD and ubiX or the homologous eukaryotic fdc1 and pad1 genes have been implicated in the non-oxidative reversible decarboxylation. The combined action of phenylacrylic acid decarboxylase (PAD) and ferulic acid decarboxylase (FDC) is considered to be essential for the decarboxylation of phenylacrylic acid in Saccharomyces cerevisiae (J. Biosci. Bioeng. 109, (2010), 564-569; AMB Express, 5:12 (2015) 1-5; ACS Chem. Biol. 10 (2015), 1137-1144).

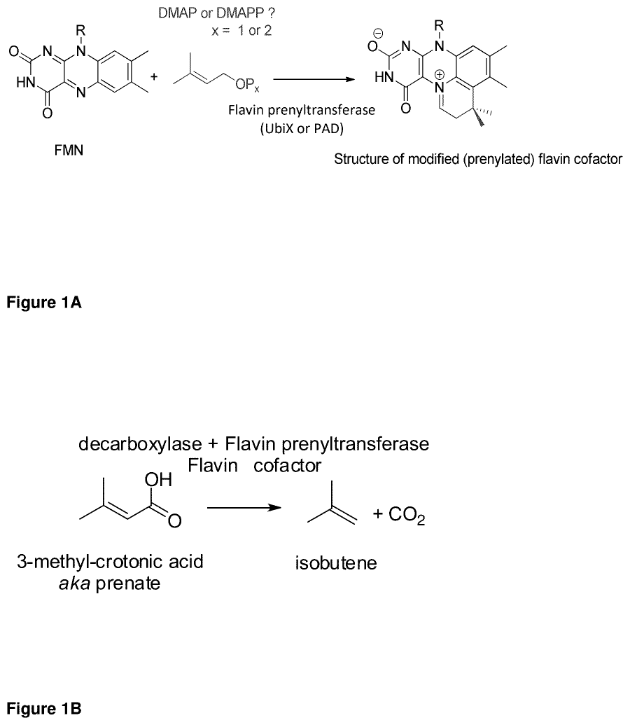

Recently, the above enzyme family described as phenylacrylic acid decarboxylase (PAD) was characterized as a Flavin prenyltransferase and no longer as a decarboxylase. It has been shown that Fdc1 (but not PAD) is solely responsible for the reversible decarboxylase activity and that it requires a new type of cofactor, namely a prenylated flavin synthesized by the associated UbiX (or Pad1) protein. Thus, the real enzymatic activity of this PAD enzyme has been identified as the transformation of a flavin mononucleotide (FMN) cofactor with a prenyl moiety (from di-methyl-allyl-phosphate or pyrophosphate called DMAP or DMAPP). This reaction is shown in FIG. 1A.

Accordingly, in contrast to the prior art's belief, the real decarboxylase is the Ferulic Acid Decarboxylase (FDC) in association with the modified FMN (prenylated-FMN). This reaction is shown in FIG. 1B. This mechanism of the Ferulic Acid Decarboxylase (FDC) in association with the modified FMN (prenylated-FMN) (the latter provided by the PAD enzyme) was recently described and involves a surprising enzymatic mechanism, i.e., an α,β-unsaturated acid decarboxylation via a 1,3-dipolar cyclo-addition. Moreover, the structure of this FDC decarboxylase has recently been elucidated (Nature 522 (2015), 497-501; Nature, 522 (2015), 502-505; Appl. Environ. Microbiol. 81 (2015), 4216-4223).

WO2017/191239 and WO2020/007886 describe enzyme variants based on FDC enzymes of Hypocrea atroviridis and Streptomyces sp. 769 which show an improved ability to convert 3-methylcrotonic acid into isobutene.

Although the above means and methods allow to produce isobutene from 3-methylcrotonic acid, there is still a need for improvements, in particular as regards a further increase in efficiency of the process so as to make it more suitable for industrial purposes.

The present application addresses this need by providing the embodiments as defined in the claims.

Thus, the present invention provides a variant of a 3-methylcrotonic acid decarboxylase (MDC) showing an improved activity in converting 3-methylcrotonic acid into isobutene over the corresponding MDC from which it is derived as defined in the claims.

An improved enzyme variant or an enzyme variant capable of catalyzing a reaction with increased activity is defined as an enzyme variant which differs from the wildtype enzyme and which catalyzes the conversion of 3-methylcrotonic acid into isobutene so that the specific activity of the enzyme variant is higher than the specific activity of the wildtype enzyme for at least one given concentration of a 3-methylcrotonic acid (preferably any 3-methylcrotonic acid higher than 0 M and up to 1 M). A specific activity is defined as the number of moles of substrate converted to moles of product by unit of time by mole of enzyme. K_{cat} (turnover number) is the specific activity at saturating concentration of substrate.

In the context of the present invention, an “improved activity” means that the activity of the enzyme in question is at least 10%, preferably at least 20%, more preferably at least 30% or 50%, even more preferably at least 70% or 80% and particularly preferred at least 90% or 100% higher than that of the enzyme from which the variant is derived, preferably higher than that of the enzyme represented by SEQ ID NO:1. In even more preferred embodiments the improved activity may be at least 150%, at least 200%, at least 300%, at least 750% or at least 1000% higher than that of the corresponding enzyme from which the variant is derived, preferably higher than that of the enzyme represented by SEQ ID NO:1. In a particularly preferred embodiment, the activity is measured by using an assay with purified enzyme and chemically synthesized substrates, as described below. The improved activity of a variant can be measured as a higher isobutene production in a given time under defined conditions, compared with the parent enzyme. This improved activity can result from a higher turnover number, e.g. a higher kcat value. It can also result from a lower Km value. It can also result from a higher kcat/Km value. Finally, it can result from a higher solubility, or stability of the enzyme. The degree of improvement can be measured as the improvement in isobutene production. The degree of improvement can also be measured in terms of kcat improvement, of kcat/Km improvement, or in terms of Km decrease, in terms of soluble protein production or in terms of protein stability.

In another embodiment, the enzyme variants which the present invention provides are capable of converting 3-methylcrotonic acid into isobutene with an activity which is at least 1.10 times as high compared to the turnover rate of the corresponding wild type enzyme having the amino acid sequence as shown in SEQ ID NO:1. In a more preferred embodiment, the enzyme variants which are capable of converting 3-methylcrotonic acid into isobutene have a turnover rate (i.e., a k_{cat}-value) which is at least 2 times, at least 3 times, at least 5 times or even at least 10 times as high compared to the turnover rate of the corresponding wild type enzyme having the amino acid sequence as shown in SEQ ID NO:1. In even more preferred embodiments, the turnover rate is at least 100 times or even at least 500 times as high compared to that of the corresponding wild type enzyme having the amino acid sequence as shown in SEQ ID NO:1.

Such enzyme variants are obtained by effecting mutations at specific positions in the amino acid sequence of an MDC and the variants obtained by effecting such mutations show an improved activity in catalyzing the conversion of 3-methylcrotonic acid into isobutene. The activity of an enzyme capable of converting 3-methylcrotonic acid into isobutene may be determined by methods known to the person skilled in the art. In one embodiment, this activity is determined as described in the Examples appended hereto. In a particular embodiment this activity can be measured by incubating the enzyme, preferably a cell lysate containing the overexpressed recombinant protein, in vitro. Alternatively, a purified enzyme can be used or an in vivo assay.

More specifically, the activity of the MDC variants for the conversion of 3-methylcrotonic acid into isobutene can be assessed by an enzymatic in vitro assay based on purified proteins and on the detection of isobutene by gas chromatography.

The MDC variant to be tested can be provided according to the following protocol: The MDC to be tested is subcloned into the pET25 (Novagen) expression vector or fused with a polynucleotide tag in 5′ or 3′ coding for a 6His purification tag before being cloned in a pET25 expression vector. The assay is based on the use of a bacterial strain (BL21 (DE3), Novagen) transformed with two expression vectors leading to the production of the last two enzymes involved in the metabolic pathway converting 3-methylcrotonic acid (3MC) to isobutene; namely the Flavin prenyltransferase UbiX protein from E. coli cloned in a pRSFDuet™ (Novagen) expression vector and the considered MDC variant cloned in one of the above expression vectors. This strain is first plated out onto LB-agar plates supplemented with the appropriate antibiotics. Cells are grown overnight at 32° C. until individual colonies reach the desired size. Single colonies are then picked and individually transferred into 50 μL of liquid LB medium supplemented with the appropriate antibiotic. Cell growth is carried out with shaking for 21 hours at 34° C. The LB cultures are used to inoculate 300 μL in 384 deepwell microplates of auto-induction medium (Studier F W, Prat. Exp. Pur. 41, (2005), 207-234) supplemented with the appropriate antibiotics and grown in a shaking incubator set at 700 rpm and 85% humidity for 24 h at 34° C. in order to produce the two types of recombinant enzymes. The cell pellet containing these two overexpressed recombinant enzymes is then resuspended in 30 μL of lysis mix (pH 7.5, Phosphate 50 mM, NaCl 20 mM, MgCl₂ 2 mM, Lysozyme 1 mg/mL, DNAse 0.03 mg/mL) and incubated for 1 hour in a shaking incubator at 34° C., 700 rpm. The mix is then supplemented with 10 μL of reaction mix (final composition: pH 7.5, Phosphate 50 mM, NaCl 20 mM, MgCl₂ 2 mM, Lysozyme 0.75 mg/mL, DNAse 0.0225 mg/mL, KCl 100 mM) supplemented with 1 to 200 mM (final) 3MC and incubated for a further 1 to 4 hours in a shaking incubator at 34° C., 700 rpm. During this step, the MDC enzyme catalyzes the decarboxylation of 3MC into isobutene (IBN). After 5 to 10 min inactivation at 80° C. or 90° C., the IBN produced is quantified by gas chromatography as followed. 100 μL of headspace gases from each enzymatic reaction are injected in a Brucker GC-450 system equipped with a Flame Ionization Detector (FID). Compounds present in samples are separated by chromatography using a RTX-1 columns at 100° C. with a 1 mL·min⁻¹ constant flow of nitrogen as carrier gas. Upon injection, peak areas of isobutene are calculated.

By providing the above described enzyme variant, the present invention allows to dramatically increase the production efficiency of isobutene from 3-methylcrotonic acid.

The term “3-methylcrotonic acid decarboxylase (MDC)” refers to an enzyme which can catalyze the decarboxylation of 3-methylcrotonic acid into isobutene. A decarboxylation is a chemical reaction that removes a carboxyl group and releases carbon dioxide. This activity can be measured by methods known in the art and as described above. In a preferred embodiment, the MDC is a Ferulic Acid Decarboxylase (FDC) or is derived from such an enzyme. FDCs belong to the enzyme class EC 4.1.1-. As mentioned above, it has originally been described that an FDC in association with a modified FMN (prenylated-FMN) is capable of catalyzing an α,β-unsaturated decarboxylation via a 1,3-dipolar cyclo-addition and, more specifically, capable of catalyzing the decarboxylation of 3-methylcrotonic acid into isobutene. Thus, in the context of the present invention, the term FDC relates to enzymes capable of catalyzing the decarboxylation of 3-methylcrotonic acid into isobutene, preferably when provided with a prenylated FMN. In a preferred embodiment the enzyme can be classified as an UbiD-like enzyme, meaning that the enzymatic activity is the same as UbiD (decarboxylation of α,β-unsaturated carboxylic acid using the same cofactor), but the substrate preference is different. An FDC is not involved in ubiquinone biosynthesis, unlike UbiD.

FDC enzymes have, e.g., been described in Saccharomyces cerevisiae, Enterobacter sp., Bacillus pumilus, Aspergillus niger or Candida dubliniensis. Hence, in preferred embodiments, the FDC is derived from Saccharomyces cerevisiae (Uniprot accession number Q03034), Enterobacter sp. (Uniprot accession number V3P7U0), Bacillus pumilus (Uniprot accession number Q45361), Aspergillus niger (Uniprot accession number A2R0P7) or Candida dubliniensis (Uniprot accession number B9WJ66). In more preferred embodiments, the FDC is a 3-polyprenyl-4-hydroxybenzoate decarboxylase (UbiD). 3-polyprenyl-4-hydroxybenzoate decarboxylases have, e.g., been described in Hypocrea atroviridis, Sphaerulina musiva, Penecillinum requeforti, Fusarium oxysporum f. sp. lycopersici, Saccharomyces kudriavzevii, Saccaromyces cerevisiae, Aspergillus parasiticus, Candida albicans, Grosmannia clavigera, Escherichia coli, Bacillus megaterium, Methanothermobacter sp. CaT2 or Mycobacterium chelonae 1518. Hence, in more preferred embodiments, the FDC enzyme variant capable of catalyzing the decarboxylation of 3-methylcrotonic acid into isobutene is derived from a 3-polyprenyl-4-hydroxybenzoate decarboxylase (UbiD) from Hypocrea atroviridis (UniProt Accession number G9NLP8), Sphaerulina musiva (UniProt Accession number M3DF95), Penecillinum roqueforti (UniProt Accession number W6QKP7), Fusarium oxysporum f. sp. lycopersici (UniProt Accession number W9LTH3), Saccharomyces kudriavzevii (UniProt Accession number J8TRN5), Saccharomyces cerevisiae, Aspergillus parasiticus, Candida albicans, Grosmannia clavigera, Escherichia coli (Uniprot accession number POAAB4), Bacillus megaterium (Uniprot accession number D5DTL4), Methanothermobacter sp. CaT2 (Uniprot accession number T2GKK5) or Mycobacterium chelonae 1518 (Uniprot accession number X8EX86). Preferably, the MDC is an enzyme which is associated with and/or depends on a Flavin prenyltransferase. As mentioned above, the enzymatic conversion of 3-methylcrotonic acid into isobutene utilizing a prenylated FMN-dependent decarboxylase is preferably associated with a Flavin prenyltransferase and relies on a reaction of two consecutive steps catalyzed by the two enzymes, i.e., the prenylated FMN-dependent decarboxylase (catalyzing the actual decarboxylation of 3-methylcrotonic acid into isobutene) with an associated Flavin prenyltransferase which provides the modified flavin cofactor. The flavin cofactor may preferably be FMN or FAD. FMN (flavin mononucleotide; also termed riboflavin-5′-phosphate) is a biomolecule produced from riboflavin (vitamin B2) by the enzyme riboflavin kinase and functions as prosthetic group of various reactions. FAD (flavin adenine dinucleotide) is a redox cofactor, more specifically a prosthetic group, involved in several important reactions in metabolism. The Flavin prenyltransferases which may be associated with the MDC variants of the present invention are described in more detail further below.

The present invention provides now improved variants of enzymes which are capable of converting 3-methylcrotonic acid into isobutene. The inventors used as a model enzyme the FDC of Yersinia frederiksenii shown in SEQ ID NO: 1 and could show that it is possible to provide variants of this enzyme which show increased activity with respect to the conversion of 3-methylcrotonic acid into isobutene.

The model enzyme, i.e., the FDC of Yersinia frederiksenii, as used by the inventors has the amino acid sequence as shown in SEQ ID NO:1.

In one preferred embodiment the variants of the present invention are characterized by the feature that they are derived from an MDC, more preferably from an MDC having the amino acid sequence shown in SEQ ID NO:1 or a highly related sequence (at least 55% identical) and in which mutations are effected at one or more of the above indicated positions and by the feature that they show the ability to convert 3-methylcrotonic acid into isobutene and that they can do this with an improved activity. In a preferred embodiment the variant according to the present invention is derived from a sequence which shows at least 60%, more preferably at least 70%, even more preferably at least 80% sequence identity to SEQ ID NO:1 and in which one or more substitutions and/or deletions and/or insertions at the positions indicated herein have been effected.

However, the teaching of the present invention is not restricted to the MDC enzyme of Yersinia frederiksenii shown in SEQ ID NO: 1 which had been used as a model enzyme but can be extended to MDC enzymes from other organisms or to enzymes which are structurally related to SEQ ID NO:1 such as, e.g., truncated variants of the enzyme. Thus, the present invention also relates to variants of MDCs which are structurally related to the Yersinia frederiksenii sequence (SEQ ID NO: 1) and which show one or more substitutions and/or deletions and/or insertions at positions corresponding to any of the positions as indicated herein below. The term “structurally related” refers to MDCs which show a sequence identity of at least n % to the sequence shown in SEQ ID NO: 1 with n being an integer between 55 and 100, preferably 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99. In a preferred embodiment the structurally related MDC stems from a bacterium, more preferably from a gram-negative bacterium, even more preferably of the genus Yersinia.

Thus, in one embodiment, the variant of an MDC according to the present invention has (or preferably is derived from) a sequence which is at least n % identical to SEQ ID NO: 1 with n being an integer between 55 and 100, preferably 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99, and it has (a) substitution(s) and/or (a) deletion(s) and/or (an) insertion(s) at a position as indicated herein. When the sequences which are compared do not have the same length, the degree of identity either refers to the percentage of amino acid residues in the shorter sequence which are identical to amino acid residues in the longer sequence or to the percentage of amino acid residues in the longer sequence which are identical to amino acid residues in the shorter sequence. Preferably, it refers to the percentage of amino acid residues in the shorter sequence which are identical to amino acid residues in the longer sequence. The degree of sequence identity can be determined according to methods well known in the art using preferably suitable computer algorithms such as CLUSTAL.

When using the Clustal analysis method to determine whether a particular sequence is, for instance, at least 55% identical to a reference sequence default settings may be used or the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap. Preferably, the degree of identity is calculated over the complete length of the sequence.

Amino acid residues located at a position corresponding to a position as indicated herein in the amino acid sequence shown in SEQ ID NO:1 can be identified by the skilled person by methods known in the art. For example, such amino acid residues can be identified by aligning the sequence in question with the sequence shown in SEQ ID NO:1 and by identifying the positions which correspond to the above or below indicated positions of SEQ ID NO:1. The alignment can be done with means and methods known to the skilled person, e.g. by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences.

In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

When the amino acid sequences of MDCs are aligned by means of such a method, regardless of insertions or deletions that occur in the amino acid sequences, the positions of the corresponding amino acid residues can be determined in each of the MDCs.

In the context of the present invention, “substituted with another amino acid residue” means that the respective amino acid residues at the indicated position can be substituted with any other possible amino acid residues, e.g. naturally occurring amino acids or non-naturally occurring amino acids (Brustad and Arnold, Curr. Opin. Chem. Biol. 15 (2011), 201-210), preferably with an amino acid residue selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Preferred substitutions for certain positions are indicated further below. Moreover, the term “substituted” or “substitution” also means that the respective amino acid residue at the indicated position is modified.

Such modifications include naturally occurring modifications and non-naturally occurring modifications. Naturally occurring modifications include but are not limited to eukaryotic post-translational modification, such as attachment of functional groups (e.g. acetate, phosphate, hydroxyl, lipids (myristoylation of glycine residues) and carbohydrates (e.g. glycosylation of arginine, asparagine etc.). Naturally occurring modifications also encompass the change in the chemical structure by citrullination, carbamylation and disulphide bond formation between cysteine residues; attachment of co-factors (FMN or FAD that can be covalently attached) or the attachment of peptides (e.g. ubiquitination or sumoylation).

Non-naturally occurring modifications include, e.g., in vitro modifications such as biotinylation of lysine residue or the inclusion of non-canonical amino acids (see Liu and Schultz, Annu. Rev. Biochem. 79 (2010), 413-44 and Wang et al., Chem. Bio. 2009 Mar. 27; 16 (3), 323-336; doi:101016/jchembiol.2009.03.001).

In the context of the present invention, “deleted” or “deletion” means that the amino acid at the corresponding position is deleted.

In the context of the present invention, “inserted” or “insertion” means that at the respective position one or two, preferably one amino acid residue is inserted after the indicated position.

The present invention provides a variant of a 3-methylcrotonic acid decarboxylase (MDC) showing an improved activity in converting 3-methylcrotonic acid into isobutene over the corresponding MDC from which it is derived, wherein the MDC variant is characterized in that it shows one or more substitutions, deletions and/or insertions in comparison to the corresponding sequence from which it is derived and wherein these substitutions, deletions and/or insertions occur at one or more of the positions corresponding to positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470 and 471 in the amino acid sequence shown in SEQ ID NO:1.

The present invention relates in a preferred embodiment to an MDC variant having an amino acid sequence as shown in SEQ ID NO:1 or an amino acid sequence having at least 55% sequence identity to SEQ ID NO:1, in which one or more amino acid residues at a position selected from the group consisting of positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470 and 471 in the amino acid sequence shown in SEQ ID NO:1 or at a position corresponding to any of these positions, are substituted with another amino acid residue or deleted or wherein an insertion has been effected at one or more of these positions and wherein said MDC variant has an improved activity in converting 3-methylcrotonic acid into isobutene.

According to one embodiment, the present invention relates to any of the above-described MDC variants having an amino acid sequence as shown in SEQ ID NO:1 or an amino acid sequence having at least 55% sequence identity to SEQ ID NO:1 in which
(1) an amino acid residue at position 7 in the amino acid sequence shown in SEQ ID NO: 1 or at a position corresponding to this position, is deleted or substituted with cysteine; and/or
(2) an amino acid residue at position 17 in the amino acid sequence shown in SEQ ID NO: 1 or at a position corresponding to this position, is deleted or substituted with serine; and/or
(3) an amino acid residue at position 27 in the amino acid sequence shown in SEQ ID NO: 1 or at a position corresponding to this position, is deleted or substituted with asparagine; and/or
(4) an amino acid residue at position 33 in the amino acid sequence shown in SEQ ID NO: 1 or at a position corresponding to this position, is deleted or substituted with proline; and/or
(5) an amino acid residue at position 35 in the amino acid sequence shown in SEQ ID NO: 1 or at a position corresponding to this position, is deleted or substituted with valine; and/or
(6) an amino acid residue at position 45 in the amino acid sequence shown in SEQ ID NO: 1 or at a position corresponding to this position, is deleted or substituted with alanine or glutamine; and/or
(7) an amino acid residue at position 46 in the amino acid sequence shown in SEQ ID NO: 1 or at a position corresponding to this position, is deleted or substituted with alanine or isoleucine; and/or
(8) an amino acid residue at position 48 in the amino acid sequence shown in SEQ ID NO: 1 or at a position corresponding to this position, is deleted or substituted with methionine; and/or
(9) an amino acid residue at position 51 in the amino acid sequence shown in SEQ ID NO: 1 or at a position corresponding to this position, is deleted or substituted with aspartate; and/or
(10) an amino acid residue at position 140 in the amino acid sequence shown in SEQ ID NO: 1 or at a position corresponding to this position, is deleted or substituted with leucine; and/or
(11) an amino acid residue at position 144 in the amino acid sequence shown in SEQ ID NO: 1 or at a position corresponding to this position, is deleted or substituted with serine; and/or
(12) an amino acid residue at position 183 in the amino acid sequence shown in SEQ ID NO: 1 or at a position corresponding to this position, is deleted or substituted with tyrosine; and/or
(13) an amino acid residue at position 184 in the amino acid sequence shown in SEQ ID NO: 1 or at a position corresponding to this position, is deleted or substituted with alanine or serine; and/or
(14) an amino acid residue at position 185 in the amino acid sequence shown in SEQ ID NO: 1 or at a position corresponding to this position, is deleted or substituted with threonine; and/or
(15) an amino acid residue at position 122 in the amino acid sequence shown in SEQ ID NO: 1 or at a position corresponding to this position, is deleted or substituted with valine; and/or
(16) an amino acid residue at position 227 in the amino acid sequence shown in SEQ ID NO: 1 or at a position corresponding to this position, is deleted or substituted with leucine or tyrosine; and/or
(17) an amino acid residue at position 284 in the amino acid sequence shown in SEQ ID NO: 1 or at a position corresponding to this position, is deleted or substituted with aspartate or glutamate or a proline is inserted; and/or
(18) an amino acid residue at position 285 in the amino acid sequence shown in SEQ ID NO: 1 or at a position corresponding to this position, is deleted or substituted with alanine, histidine, isoleucine, leucine, methionine, proline, serine, threonine or valine; and/or
(19) an amino acid residue at position 286 in the amino acid sequence shown in SEQ ID NO: 1 or at a position corresponding to this position, is deleted or substituted with alanine, cysteine, aspartate, glycine, histidine, asparagine, serine or tryptophan; and/or
(20) an amino acid residue at position 287 in the amino acid sequence shown in SEQ ID NO: 1 or at a position corresponding to this position, is deleted or substituted with isoleucine, lysine, proline, glutamine, arginine, serine, threonine or valine or a serine is inserted; and/or
(21) an amino acid residue at position 288 in the amino acid sequence shown in SEQ ID NO: 1 or at a position corresponding to this position, is deleted or substituted with asparagine or threonine; and/or
(22) an amino acid residue at position 289 in the amino acid sequence shown in SEQ ID NO: 1 or at a position corresponding to this position, is deleted or substituted with alanine, cysteine, histidine, methionine, glutamine, arginine, tryptophan or tyrosine; and/or
(23) an amino acid residue at position 290 in the amino acid sequence shown in SEQ ID NO: 1 or at a position corresponding to this position, is deleted or substituted with glutamate; and/or
(24) an amino acid residue at position 292 in the amino acid sequence shown in SEQ ID NO: 1 or at a position corresponding to this position, is deleted or substituted with leucine or valine; and/or
(25) an amino acid residue at position 321 in the amino acid sequence shown in SEQ ID NO: 1 or at a position corresponding to this position, is deleted or substituted with threonine or valine; and/or
(26) an amino acid residue at position 322 in the amino acid sequence shown in SEQ ID NO: 1 or at a position corresponding to this position, is deleted or substituted with threonine; and/or
(27) an amino acid residue at position 327 in the amino acid sequence shown in SEQ ID NO: 1 or at a position corresponding to this position, is deleted or substituted with serine; and/or
(28) an amino acid residue at position 329 in the amino acid sequence shown in SEQ ID NO: 1 or at a position corresponding to this position, is deleted or substituted with serine; and/or
(29) an amino acid residue at position 330 in the amino acid sequence shown in SEQ ID NO: 1 or at a position corresponding to this position, is deleted or substituted with valine; and/or
(30) an amino acid residue at position 331 in the amino acid sequence shown in SEQ ID NO: 1 or at a position corresponding to this position, is deleted or substituted with glycine; and/or
(30) an amino acid residue at position 331 in the amino acid sequence shown in SEQ ID NO: 1 or at a position corresponding to this position, is deleted or substituted with glycine; and/or
(31) an amino acid residue at position 337 in the amino acid sequence shown in SEQ ID NO: 1 or at a position corresponding to this position, is deleted or substituted with threonine; and/or
(32) an amino acid residue at position 338 in the amino acid sequence shown in SEQ ID NO: 1 or at a position corresponding to this position, is deleted or substituted with glycine; and/or
(33) an amino acid residue at position 355 in the amino acid sequence shown in SEQ ID NO: 1 or at a position corresponding to this position, is deleted or substituted with cysteine, threonine or valine; and/or
(34) an amino acid residue at position 365 in the amino acid sequence shown in SEQ ID NO: 1 or at a position corresponding to this position, is deleted or substituted with isoleucine, methionine, serine or threonine; and/or
(35) an amino acid residue at position 378 in the amino acid sequence shown in SEQ ID NO: 1 or at a position corresponding to this position, is deleted or substituted with methionine; and/or
(36) an amino acid residue at position 380 in the amino acid sequence shown in SEQ ID NO: 1 or at a position corresponding to this position, is deleted or substituted with valine; and/or
(37) an amino acid residue at position 384 in the amino acid sequence shown in SEQ ID NO: 1 or at a position corresponding to this position, is deleted or substituted with alanine, glycine, leucine, serine or threonine; and/or
(38) an amino acid residue at position 387 in the amino acid sequence shown in SEQ ID NO: 1 or at a position corresponding to this position, is deleted or substituted with alanine; and/or
(39) an amino acid residue at position 388 in the amino acid sequence shown in SEQ ID NO: 1 or at a position corresponding to this position, is deleted or substituted with methionine, glutamine or serine; and/or
(40) an amino acid residue at position 389 in the amino acid sequence shown in SEQ ID NO: 1 or at a position corresponding to this position, is deleted or substituted with alanine, histidine or glutamine; and/or
(41) an amino acid residue at position 391 in the amino acid sequence shown in SEQ ID NO: 1 or at a position corresponding to this position, is deleted or substituted with isoleucine or leucine; and/or
(42) an amino acid residue at position 392 in the amino acid sequence shown in SEQ ID NO: 1 or at a position corresponding to this position, is deleted or substituted with tyrosine; and/or
(43) an amino acid residue at position 393 in the amino acid sequence shown in SEQ ID NO: 1 or at a position corresponding to this position, is deleted or substituted with alanine; and/or
(44) an amino acid residue at position 394 in the amino acid sequence shown in SEQ ID NO: 1 or at a position corresponding to this position, is deleted or substituted with alanine, cysteine, glycine, histidine, leucine, glutamine or arginine; and/or
(45) an amino acid residue at position 395 in the amino acid sequence shown in SEQ ID NO: 1 or at a position corresponding to this position, is deleted or substituted with valine; and/or
(46) an amino acid residue at position 419 in the amino acid sequence shown in SEQ ID NO: 1 or at a position corresponding to this position, is deleted or substituted with alanine or proline; and/or
(47) at position 424 in the amino acid sequence shown in SEQ ID NO:1 or at a position corresponding to this position, an alanine, aspartate, leucine, serine or tryptophan is inserted; and/or
(48) at position 425 in the amino acid sequence shown in SEQ ID NO:1 or at a position corresponding to this position, a tryptophan is inserted.
(49) an amino acid residue at position 428 in the amino acid sequence shown in SEQ ID NO: 1 or at a position corresponding to this position, is deleted or substituted with methionine or threonine; and/or
(50) an amino acid residue at position 429 in the amino acid sequence shown in SEQ ID NO: 1 or at a position corresponding to this position, is deleted or substituted with alanine, aspartate or valine or a glycine or an asparagine is inserted; and/or
(51) an amino acid residue at position 431 in the amino acid sequence shown in SEQ ID NO: 1 or at a position corresponding to this position, is deleted or substituted with alanine, cysteine, lysine, methionine, asparagine, proline, threonine or valine or a glycine, leucine or asparagine is inserted; and/or
(52) an amino acid residue at position 432 in the amino acid sequence shown in SEQ ID NO: 1 or at a position corresponding to this position, is deleted or substituted with alanine, phenylalanine or tryptophan; and/or
(53) an amino acid residue at position 434 in the amino acid sequence shown in SEQ ID NO: 1 or at a position corresponding to this position, is deleted or substituted with phenylalanine, methionine, valine or tryptophan; and/or
(54) an amino acid residue at position 436 in the amino acid sequence shown in SEQ ID NO: 1 or at a position corresponding to this position, is deleted or substituted with alanine, methionine, asparagine, glutamine, threonine or tryptophan; and/or
(55) an amino acid residue at position 437 in the amino acid sequence shown in SEQ ID NO: 1 or at a position corresponding to this position, is deleted or substituted with phenylalanine, glycine, isoleucine, leucine, methionine, threonine or valine; and/or
(56) an amino acid residue at position 438 in the amino acid sequence shown in SEQ ID NO: 1 or at a position corresponding to this position, is deleted or substituted with leucine, methionine or tryptophan or an aspartate is inserted; and/or
(57) an amino acid residue at position 439 in the amino acid sequence shown in SEQ ID NO: 1 or at a position corresponding to this position, is deleted or substituted with leucine or proline; and/or
(58) an amino acid residue at position 443 in the amino acid sequence shown in SEQ ID NO: 1 or at a position corresponding to this position, is deleted or substituted with histidine or an alanine, glycine, leucine, arginine, threonine or valine is inserted; and/or
(59) an amino acid residue at position 444 in the amino acid sequence shown in SEQ ID NO: 1 or at a position corresponding to this position, is deleted or substituted with phenylalanine, isoleucine, leucine, methionine, asparagine, arginine, threonine or valine or a glutamate, glycine, histidine, lysine, leucine, asparagine, proline, arginine, serine, threonine or valine is inserted; and/or
(60) an amino acid residue at position 446 in the amino acid sequence shown in SEQ ID NO: 1 or at a position corresponding to this position, is deleted or substituted with aspartate, glycine or proline; and/or
(61) an amino acid residue at position 447 in the amino acid sequence shown in SEQ ID NO: 1 or at a position corresponding to this position, is deleted or substituted with phenylalanine, histidine, isoleucine, threonine or tryptophan or a lysine, leucine, proline or tyrosine is inserted; and/or
(62) an amino acid residue at position 448 in the amino acid sequence shown in SEQ ID NO: 1 or at a position corresponding to this position, is deleted or substituted with cysteine, glutamate, threonine or valine or a serine is inserted; and/or
(63) an amino acid residue at position 449 in the amino acid sequence shown in SEQ ID NO: 1 or at a position corresponding to this position, is deleted or substituted with asparagine or tryptophan valine or a serine is inserted; and/or
(64) an amino acid residue at position 451 in the amino acid sequence shown in SEQ ID NO: 1 or at a position corresponding to this position, is deleted or substituted with serine; and/or
(65) an amino acid residue at position 453 in the amino acid sequence shown in SEQ ID NO: 1 or at a position corresponding to this position, is deleted or substituted with leucine; and/or
(66) an amino acid residue at position 457 in the amino acid sequence shown in SEQ ID NO: 1 or at a position corresponding to this position, is deleted or substituted with alanine; and/or
(67) an amino acid residue at position 458 in the amino acid sequence shown in SEQ ID NO: 1 or at a position corresponding to this position, is deleted or substituted with cysteine, glutamate, isoleucine, leucine, methionine, glutamine or arginine or a glycine is inserted; and/or
(68) an amino acid residue at position 459 in the amino acid sequence shown in SEQ ID NO: 1 or at a position corresponding to this position, is deleted or substituted with glycine, leucine, proline, arginine or threonine; and/or
(69) an amino acid residue at position 460 in the amino acid sequence shown in SEQ ID NO: 1 or at a position corresponding to this position, is deleted or substituted glycine, histidine, isoleucine, leucine, methionine, valine or tyrosine; and/or
(70) an amino acid residue at position 462 in the amino acid sequence shown in SEQ ID NO: 1 or at a position corresponding to this position, is deleted or substituted with alanine, cysteine, isoleucine, proline, arginine, threonine or valine or an alanine is inserted; and/or
(71) an amino acid residue at position 464 in the amino acid sequence shown in SEQ ID NO: 1 or at a position corresponding to this position, is deleted or substituted with proline; and/or
(72) an amino acid residue at position 465 in the amino acid sequence shown in SEQ ID NO: 1 or at a position corresponding to this position, is deleted or substituted with alanine, isoleucine or valine; and/or
(73) an amino acid residue at position 467 in the amino acid sequence shown in SEQ ID NO: 1 or at a position corresponding to this position, is deleted or substituted with tryptophan; and/or
(74) an amino acid residue at position 468 in the amino acid sequence shown in SEQ ID NO: 1 or at a position corresponding to this position, is deleted or substituted with leucine; and/or
(75) an amino acid residue at position 470 in the amino acid sequence shown in SEQ ID NO: 1 or at a position corresponding to this position, is deleted or substituted with glycine, serine or threonine; and/or
(76) an amino acid residue at position 471 in the amino acid sequence shown in SEQ ID NO: 1 or at a position corresponding to this position, is deleted or substituted with phenylalanine.

The invention also relates to variants as defined in (1) to (76) hereinabove, wherein the amino acid residue indicated as substituting the amino acid residue at the position in SEQ ID NO: 1 or as being inserted at a certain position is not that particular amino acid residue but an amino acid residue which is conservative in relation to the indicated substituting amino acid.

Whether an amino acid is conservative with respect to another amino acid can be judged according to means and methods known in the art and as described herein above. One possibility is the PAM 250 matrix; alternatively, the Blosum Family Matrices can be used.

The present invention also relates to an MDC variant as described herein above which has an amino acid sequence as shown in SEQ ID NO:1 or an amino acid sequence having at least 55% sequence identity to SEQ ID NO:1, in which one or more amino acid residues at a position selected from the group consisting of positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470 and 471 in the amino acid sequence shown in SEQ ID NO:1 or at a position corresponding to any of these positions, are substituted with another amino acid residue or deleted or wherein an insertion has been effected at one or more of these positions and which furthermore shows at least one modification at a position selected from the group consisting of 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463 and 469 in the amino acid sequence shown in SEQ ID NO:1.

According to one embodiment, such an MDC variant as described herein above which furthermore shows at least one modification at a position selected from the group consisting of 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463 and 469 in the amino acid sequence shown in SEQ ID NO:1 or at a position corresponding to any of these positions is an MDC variant, wherein
(1) an amino acid residue at position 6 in the amino acid sequence shown in SEQ ID NO: 1 or at a position corresponding to this position, is deleted or substituted with methionine; and/or
(2) an amino acid residue at position 15 in the amino acid sequence shown in SEQ ID NO: 1 or at a position corresponding to this position, is deleted or substituted with histidine or leucine; and/or
(3) an amino acid residue at position 25 in the amino acid sequence shown in SEQ ID NO: 1 or at a position corresponding to this position, is deleted or substituted with leucine; and/or
(4) an amino acid residue at position 39 in the amino acid sequence shown in SEQ ID NO: 1 or at a position corresponding to this position, is deleted or substituted with glycine; and/or
(5) an amino acid residue at position 43 in the amino acid sequence shown in SEQ ID NO: 1 or at a position corresponding to this position, is deleted or substituted with glutamate; and/or
(6) an amino acid residue at position 72 in the amino acid sequence shown in SEQ ID NO: 1 or at a position corresponding to this position, is deleted or substituted with lysine; and/or
(7) an amino acid residue at position 89 in the amino acid sequence shown in SEQ ID NO: 1 or at a position corresponding to this position, is deleted or substituted with asparagine; and/or
(8) an amino acid residue at position 126 in the amino acid sequence shown in SEQ ID NO: 1 or at a position corresponding to this position, is deleted or substituted with lysine; and/or
(9) an amino acid residue at position 141 in the amino acid sequence shown in SEQ ID NO: 1 or at a position corresponding to this position, is deleted or substituted with cysteine, leucine, glutamine, serine or tyrosine; and/or
(10) an amino acid residue at position 189 in the amino acid sequence shown in SEQ ID NO: 1 or at a position corresponding to this position, is deleted or substituted with cysteine; and/or
(11) an amino acid residue at position 223 in the amino acid sequence shown in SEQ ID NO: 1 or at a position corresponding to this position, is deleted or substituted with tyrosine; and/or
(12) an amino acid residue at position 224 in the amino acid sequence shown in SEQ ID NO: 1 or at a position corresponding to this position, is deleted or substituted with alanine, proline or glutamine; and/or
(13) an amino acid residue at position 272 in the amino acid sequence shown in SEQ ID NO: 1 or at a position corresponding to this position, is deleted or substituted with isoleucine, lysine, leucine or arginine; and/or
(14) an amino acid residue at position 283 in the amino acid sequence shown in SEQ ID NO: 1 or at a position corresponding to this position, is deleted or substituted with cysteine, phenylalanine, histidine, leucine, glutamine, serine, threonine or valine; and/or
(15) an amino acid residue at position 332 in the amino acid sequence shown in SEQ ID NO: 1 or at a position corresponding to this position, is deleted or substituted with histidine, arginine or serine; and/or
(16) an amino acid residue at position 333 in the amino acid sequence shown in SEQ ID NO: 1 or at a position corresponding to this position, is deleted or substituted with methionine; and/or
(17) an amino acid residue at position 336 in the amino acid sequence shown in SEQ ID NO: 1 or at a position corresponding to this position, is deleted or substituted with histidine, asparagine or glutamine; and/or
(18) an amino acid residue at position 361 in the amino acid sequence shown in SEQ ID NO: 1 or at a position corresponding to this position, is deleted or substituted with lysine; and/or
(19) an amino acid residue at position 3665 in the amino acid sequence shown in SEQ ID NO:1 or at a position corresponding to this position, is deleted or substituted with alanine; and/or
(20) an amino acid residue at position 368 in the amino acid sequence shown in SEQ ID NO: 1 or at a position corresponding to this position, is deleted or substituted with serine; and/or
(21) an amino acid residue at position 379 in the amino acid sequence shown in SEQ ID NO: 1 or at a position corresponding to this position, is deleted or substituted with isoleucine; and/or
(22) an amino acid residue at position 382 in the amino acid sequence shown in SEQ ID NO: 1 or at a position corresponding to this position, is deleted or substituted with alanine or leucine; and/or
(23) an amino acid residue at position 385 in the amino acid sequence shown in SEQ ID NO: 1 or at a position corresponding to this position, is deleted or substituted with aspartate, phenylalanine, glycine, leucine, proline, arginine, serine, threonine or tryptophan; and/or
(24) an amino acid residue at position 418 in the amino acid sequence shown in SEQ ID NO: 1 or at a position corresponding to this position, is deleted or substituted with valine; and/or
(25) an amino acid residue at position 421 in the amino acid sequence shown in SEQ ID NO: 1 or at a position corresponding to this position, is deleted or substituted with phenylalanine, lysine, leucine, threonine, valine or tryptophan or an aspartate, glutamate, glycine, isoleucine, proline, arginine serine or threonine is inserted; and/or
(26) an amino acid residue at position 422 in the amino acid sequence shown in SEQ ID NO: 1 or at a position corresponding to this position, is deleted or substituted with aspartate, phenylalanine, glycine, histidine, leucine, glutamine, serine or valine or a lysine is inserted; and/or
(27) an amino acid residue at position 423 in the amino acid sequence shown in SEQ ID NO: 1 or at a position corresponding to this position, is deleted or substituted with cysteine or glycine; and/or
(28) an amino acid residue at position 426 in the amino acid sequence shown in SEQ ID NO: 1 or at a position corresponding to this position, is deleted or substituted with histidine, isoleucine, leucine, threonine, valine, tryptophan or tyrosine or a leucine is inserted; and/or
(29) an amino acid residue at position 430 in the amino acid sequence shown in SEQ ID NO: 1 or at a position corresponding to this position, is deleted or substituted with alanine, lysine, asparagine, proline, arginine, serine, threonine or valine or a cysteine, glycine, histidine, isoleucine, leucine, asparagine, glutamine, arginine or serine is inserted; and/or
(30) an amino acid residue at position 440 in the amino acid sequence shown in SEQ ID NO: 1 or at a position corresponding to this position, is deleted or substituted with aspartate or isoleucine; and/or
(31) an amino acid residue at position 441 in the amino acid sequence shown in SEQ ID NO: 1 or at a position corresponding to this position, is deleted or substituted with aspartate, glycine, histidine, isoleucine, leucine, proline, threonine or valine; and/or
(32) an amino acid residue at position 445 in the amino acid sequence shown in SEQ ID NO: 1 or at a position corresponding to this position, is deleted or substituted with alanine, glutamine or threonine or a glutamine is inserted; and/or
(33) an amino acid residue at position 461 in the amino acid sequence shown in SEQ ID NO: 1 or at a position corresponding to this position, is deleted or substituted with alanine, aspartate, glutamate, glycine, asparagine, serine, threonine, valine or tyrosine; and/or
(34) an amino acid residue at position 463 in the amino acid sequence shown in SEQ ID NO: 1 or at a position corresponding to this position, is deleted or substituted with glutamate, phenylalanine, glycine, isoleucine, leucine, methionine, proline, serine, threonine or valine; and/or
(35) an amino acid residue at position 469 in the amino acid sequence shown in SEQ ID NO: 1 or at a position corresponding to this position, is deleted or substituted with alanine, aspartate, glutamate, histidine, asparagine, serine or threonine.

The invention also relates to variants as defined in (1) to (35) hereinabove, wherein the amino acid residue indicated as substituting the amino acid residue at the position in SEQ ID NO: 1 is not that particular amino acid residue but an amino acid residue which is conservative in relation to the indicated substituting amino acid.

Whether an amino acid is conservative with respect to another amino acid can be judged according to means and methods known in the art and as described herein above. One possibility is the PAM 250 matrix; alternatively, the Blosum Family Matrices can be used.

In a preferred embodiment the present invention relates in particular to a variant of a 3-methylcrotonic acid decarboxylase (MDC) showing an improved activity in converting 3-methylcrotonic acid into isobutene over the 3-methylcrotonic acid decarboxylase (MDC) of SEQ ID NO: 1, wherein the 3-methylcrotonic acid decarboxylase (MDC) is characterized in that:
(a) it has a sequence identity of at least 55% to the sequence shown in SEQ ID NO: 1; and
(b) it shows one or more substitutions at a position selected from the group consisting of positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 428, 429, 431, 432, 434, 436, 437, 438, 439, 443, 444, 446, 447, 448, 449, 451, 453, 457, 458, 459, 460, 462, 464, 465, 467, 468, 470 and 471 in the amino acid sequence shown in SEQ ID NO:1 or at a position corresponding to any of these positions; and/or
   it shows one or more deletions at a position selected from the group consisting of positions 422, 458, 459, 460, 461, 462 and 463 in the amino acid sequence shown in SEQ ID NO:1 or at a position corresponding to any of these positions; and/or
   it shows one or more insertions at a position selected from the group consisting of positions 284, 287, 421, 422, 424, 425, 426, 429, 430, 431, 438, 443, 444, 445, 447, 448, 449, 458 and 462 in the amino acid sequence shown in SEQ ID NO: 1 or at a position corresponding to any of these positions.

As regards the preferred modifications at the different positions, in particular in connection with substitutions and insertions, the same applies as has been set forth above.

In one preferred embodiment, the MDC variant according to the invention showing an improved activity in converting 3-methylcrotonic acid into isobutene is characterized in that it contains at least one deletion, substitution and/or insertion wherein the deletion/insertion/substitution is at position 432 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 434, 436, 437, 438, 439, 443, 444, 446, 447, 448, 449, 451, 453, 457, 458, 459, 460, 462, 464, 465, 467, 468, 470, 471, 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463 and 469 in the amino acid sequence shown in SEQ ID NO:1.

As regards the preferred embodiments of substitutions, deletions and/or insertions at a particular position, the same applies as has been set forth herein above.

In another preferred embodiment, the MDC variant according to the invention showing an improved activity in converting 3-methylcrotonic acid into isobutene is characterized in that it contains at least one deletion, substitution and/or insertion wherein the deletion/insertion/substitution is at position 434 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 436, 437, 438, 439, 443, 444, 446, 447, 448, 449, 451, 453, 457, 458, 459, 460, 462, 464, 465, 467, 468, 470, 471, 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463 and 469 in the amino acid sequence shown in SEQ ID NO:1.

As regards the preferred embodiments of substitutions, deletions and/or insertions at a particular position, the same applies as has been set forth herein above.

In a further preferred embodiment, the MDC variant according to the invention showing an improved activity in converting 3-methylcrotonic acid into isobutene is characterized in that it contains at least one deletion, substitution and/or insertion wherein the deletion/insertion/substitution is at position 436 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 437, 438, 439, 443, 444, 446, 447, 448, 449, 451, 453, 457, 458, 459, 460, 462, 464, 465, 467, 468, 470, 471, 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463 and 469 in the amino acid sequence shown in SEQ ID NO:1.

As regards the preferred embodiments of substitutions, deletions and/or insertions at a particular position, the same applies as has been set forth herein above.

In a further preferred embodiment, the MDC variant according to the invention showing an improved activity in converting 3-methylcrotonic acid into isobutene is characterized in that it contains at least one deletion, substitution and/or insertion wherein the deletion/insertion/substitution is at position 387 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470, 471, 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463 and 469 in the amino acid sequence shown in SEQ ID NO:1.

In a more preferred embodiment, the MDC variant according to the invention showing an improved activity in converting 3-methylcrotonic acid into isobutene is characterized in that contains at least two deletions, substitutions and/or insertions wherein one deletion/insertion/substitution is at position 432 in the amino acid sequence shown in SEQ ID NO:1 or at a position corresponding to this position and another deletion/insertion/substitution is at position 434 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 436, 437, 438, 439, 443, 444, 446, 447, 448, 449, 451, 453, 457, 458, 459, 460, 462, 464, 465, 467, 468, 470, 471, 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463 and 469 in the amino acid sequence shown in SEQ ID NO:1. It is particularly preferred that such a variant which contains modifications at positions 432 and 434 in the amino acid sequence shown in SEQ ID NO:1 or at positions corresponding to these positions furthermore shows a substitution, deletion or substitution at position 430 in the amino acid sequence shown in SEQ ID NO:1 or at a position corresponding to this position.

It is also particularly preferred that such a variant which contains modifications at positions 432 and 434 in the amino acid sequence shown in SEQ ID NO:1 or at positions corresponding to these positions furthermore shows a substitution, deletion or substitution at position 436 in the amino acid sequence shown in SEQ ID NO:1 or at a position corresponding to this position.

It is even more preferred that such a variant which contains modifications at positions 432 and 434 in the amino acid sequence shown in SEQ ID NO:1 or at positions corresponding to these positions furthermore shows a substitution, deletion or substitution at positions 430 and at position 436 in the amino acid sequence shown in SEQ ID NO:1 or at position corresponding to these positions.

As regards the preferred embodiments of substitutions, deletions and/or insertions at a particular position, the same applies as has been set forth herein above.

In one particularly preferred embodiment, the MDC variant according to the invention showing an improved activity in converting 3-methylcrotonic acid into isobutene is characterized in that it contains at least one deletion, substitution and/or insertion wherein the deletion/insertion/substitution is at position 430 in the amino acid sequence shown in SEQ ID NO:1 or at a position corresponding to this position and in that it contains deletions, substitutions and/or insertions at at least two further positions selected from the group consisting of positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470, 471, 6, 15, 25, 39, 43, 72, 89, 126, 141, 189, 223, 224, 272, 283, 332, 333, 336, 361, 366, 368, 379, 382, 385, 418, 421, 422, 423, 426, 440, 441, 445, 461, 463 and 469 in the amino acid sequence shown in SEQ ID NO:1 or at positions corresponding to these positions. In a preferred embodiment the at least two further positions are 432 and 434. In another preferred embodiment the at least two further positions are 432, 434 and 436.

As regards the preferred embodiments of substitutions, deletions and/or insertions at a particular position, the same applies as has been set forth herein above.

In preferred embodiments, the MDC variant according to the invention showing an improved activity in converting 3-methylcrotonic acid into isobutene is characterized in that it contains two or more modifications. Preferred combinations of positions at which such modifications occur are specified in the following Table 1. Thus, in preferred embodiments, the MDC variant shows modifications at two or more positions (in the amino acid sequence shown in SEQ ID NO:1 or at positions corresponding to these positions) as reflected by the positions indicated for any one of the variants with more than one modification shown in Table 1.

For Example, Table 1 shows the variant “K391|-L432W-L434M”. Accordingly, the MDC variant according to the invention in one preferred embodiment contains three modifications which occur at positions 391, 432 and 434 (in the amino acid sequence shown in SEQ ID NO:1 or at positions corresponding to these positions). Alternative combinations of positions are reflected by the other mutants which are listed in Table 1.

In more preferred embodiments, an MDC variant which contains two or more modifications at positions as indicated for any of the mutants listed in Table 1, contains the modifications as shown in Table 1.

Thus, for example, an MDC variant which shows modifications at positions 391, 432 and 434, preferably shows, in one embodiment, a substitutions from K to I at position 391, a substitution from L to W at position 432 and a substitution from L to M at position 434.

TABLE 1

| | Activity |
|---|---|
| | relative |
| Mutants | to WT |
| R387A | + |
| L432A | + |
| L434V | + |
| S436A | + |
| G380V-K391L-L432W-L434M | + |
| L432F-L434W | + |
| L432F-L434F | + |
| L432W-L434W | + |
| K391I-L432W-L434M | + |
| L432A-S436A | + |
| L432F-L434W-S436A | + |
| R387A-L432F-L434W-S436A | + |
| N388M-L432F-L434W | + |
| K394R-L432F-L434W | + |
| C430S-L432F-L434W | + |
| L432F-L434W-S436N | + |
| N388M-L432F-L434W-S436A | + |
| C430P-L432F-L434W-S436A | + |
| C430S-L432F-L434W-S436A | + |
| K394R-L432F-L434W-S436A | + |
| N388M-L432F-L434W-S436N | + |
| C430P-L432F-L434W-S436N | + |
| C430S-L432F-L434W-S436N | + |
| K394R-L432F-L434W-S436N | + |
| N388M-C430P-L432F-L434W-S436A | + |
| N388M-C430S-L432F-L434W-S436A | + |
| N388M-K394R-L432F-L434W-S436A | + |
| K394R-C430P-L432F-L434W-S436A | + |
| K394R-C430S-L432F-L434W-S436A | + |
| N388M-C430P-L432F-L434W-S436N | + |
| N388M-C430S-L432F-L434W-S436N | + |
| N388M-K394R-L432F-L434W-S436N | + |
| K394R-C430S-L432F-L434W-S436N | + |
| K394R-C430P-L432F-L434W-S436N | + |
| N388M-K394R-C430S-L432F-L434W-S436A | + |
| N388M-K394R-C430P-L432F-L434W-S436A | + |
| N388M-K394R-C430P-L432F-L434W-S436N | + |
| N388M-K394R-C430S-L432F-L434W-S436N | + |
| H141Y-N388M-K394R-C430P-L432F-L434W-S436N | + |
| N388M-K394R-C430K-L432F-L434W-S436N | + |
| N388M-K394R-C430N-L432F-L434W-S436N | + |
| N388M-K394R-I395V-C430P-L432F-L434W-S436N | + |
| N388M-C430A-L432F-L434W-S436N | + |
| N388M-K394A-C430P-L432F-L434W-S436N | + |
| N388M-K394C-C430P-L432F-L434W-S436N | + |
| N388M-K394H-C430P-L432F-L434W-S436N | + |
| N388Q-C430P-L432F-L434W-S436N | + |
| S185T-N388M-C430P-L432F-L434W-S436N | + |
| N388M-K394H-C430S-L432F-L434W-S436N | + |
| S185T-N388M-C430S-L432F-L434W-S436N | + |
| N388M-K394R-C430A-L432F-L434W-S436N | + |
| N388M-K394R-C430N-L432F-L434W-S436N-H444L | + |
| N388M-K394R-C430N-L432F-L434W-S436N-H444N | + |
| N388M-K394R-C430N-L432F-L434W-S436N-H444R | + |
| N388M-K394R-C430N-L432F-L434W-S436N-H444T | + |
| N388M-K394R-C430N-L432F-L434W-S436N-H444V | + |
| N285M-N388M-K394R-C430N-L432F-L434W-S436N | + |
| N287I-N388M-K394R-C430N-L432F-L434W-S436N | + |
| N287K-N388M-K394R-C430N-L432F-L434W-S436N | + |
| N287Q-N388M-K394R-C430N-L432F-L434W-S436N | + |
| N287R-N388M-K394R-C430N-L432F-L434W-S436N | + |
| N287T-N388M-K394R-C430N-L432F-L434W-S436N | + |
| N287V-N388M-K394R-C430N-L432F-L434W-S436N | + |
| N332H-N388M-K394R-C430N-L432F-L434W-S436N | + |
| P284E-N388M-K394R-C430N-L432F-L434W-S436N | + |
| S322T-N388M-K394R-C430N-L432F-L434W-S436N | + |
| N388M-K394R-S429D-C430N-L432F-L434W-S436N | + |
| V382A-N388M-K394R-C430N-L432F-L434W-S436N | + |
| V382L-N388M-K394R-C430N-L432F-L434W-S436N | + |
| P284E-N285M-N388M-K394R-C430N-L432F-L434W-S436N | ++ |
| P284E-N287Q-N388M-K394R-C430N-L432F-L434W-S436N | ++ |
| P284E-N287V-N388M-K394R-C430N-L432F-L434W-S436N | ++ |
| P284E-N287R-N388M-K394R-C430N-L432F-L434W-S436N | ++ |
| P284E-N287T-N388M-K394R-C430N-L432F-L434W-S436N | ++ |
| P284E-N287K-N388M-K394R-C430N-L432F-L434W-S436N | ++ |
| P284E-S322T-N388M-K394R-C430N-L432F-L434W-S436N | ++ |
| N285M-N287Q-N388M-K394R-C430N-L432F-L434W-S436N | ++ |
| N285M-N287V-N388M-K394R-C430N-L432F-L434W-S436N | ++ |
| N285M-N287R-N388M-K394R-C430N-L432F-L434W-S436N | ++ |
| N285M-N287T-N388M-K394R-C430N-L432F-L434W-S436N | ++ |
| N285M-N287K-N388M-K394R-C430N-L432F-L434W-S436N | ++ |
| N285M-S322T-N388M-K394R-C430N-L432F-L434W-S436N | ++ |
| N287Q-S322T-N388M-K394R-C430N-L432F-L434W-S436N | ++ |
| N287V-S322T-N388M-K394R-C430N-L432F-L434W-S436N | ++ |
| N287R-S322T-N388M-K394R-C430N-L432F-L434W-S436N | ++ |
| N287T-S322T-N388M-K394R-C430N-L432F-L434W-S436N | ++ |
| N287K-S322T-N388M-K394R-C430N-L432F-L434W-S436N | ++ |
| P284E-N285M-N287Q-N388M-K394R-C430N-L432F-L434W-S436N | ++ |
| P284E-N285M-N287V-N388M-K394R-C430N-L432F-L434W-S436N | ++ |
| P284E-N285M-N287Q-S322T-N388M-K394R-C430N-L432F-L434W-S436N | ++ |
| P284E-N285M-N287T-N388M-K394R-C430N-L432F-L434W-S436N | ++ |
| P284E-N285M-N287K-N388M-K394R-C430N-L432F-L434W-S436N | ++ |
| P284E-N285M-S322T-N388M-K394R-C430N-L432F-L434W-S436N | ++ |
| P284E-N287Q-S322T-N388M-K394R-C430N-L432F-L434W-S436N | ++ |
| P284E-N287V-S322T-N388M-K394R-C430N-L432F-L434W-S436N | ++ |
| P284E-N287R-S322T-N388M-K394R-C430N-L432F-L434W-S436N | ++ |
| P284E-N287T-S322T-N388M-K394R-C430N-L432F-L434W-S436N | ++ |
| P284E-N287K-S322T-N388M-K394R-C430N-L432F-L434W-S436N | ++ |
| N285M-N287Q-S322T-N388M-K394R-C430N-L432F-L434W-S436N | ++ |
| N285M-N287V-S322T-N388M-K394R-C430N-L432F-L434W-S436N | ++ |
| N285M-N287R-S322T-N388M-K394R-C430N-L432F-L434W-S436N | ++ |
| N285M-N287T-S322T-N388M-K394R-C430N-L432F-L434W-S436N | ++ |
| N285M-N287K-S322T-N388M-K394R-C430N-L432F-L434W-S436N | ++ |
| P284E-N285M-N287R-N388M-K394R-C430N-L432F-L434W-S436N | ++ |
| P284E-N285M-N287V-S322T-N388M-K394R-C430N-L432F-L434W-S436N | ++ |
| P284E-N285M-N287R-S322T-N388M-K394R-C430N-L432F-L434W-S436N | ++ |
| P284E-N285M-N287T-S322T-N388M-K394R-C430N-L432F-L434W-S436N | ++ |
| P284E-N285M-N287K-S322T-N388M-K394R-C430N-L432F-L434W-S436N | ++ |
| I283T-N285P-N287T-Q290E-S322T-N388M-K394R-C430A-L432F-L434W-S436N-dG462-dA463 | ++ |
| S189C-P284E-N287R-S322T-N388M-K394R-C430N-L432F-L434W-S436N | +++ |
| I283S-P284E-N287R-S322T-N388M-K394R-C430N-L432F-L434W-S436N | +++ |
| I283F-P284E-N287R-S322T-N388M-K394R-C430N-L432F-L434W-S436N | +++ |
| I283H-P284E-N287R-S322T-N388M-K394R-C430N-L432F-L434W-S436N | +++ |
| I283V-P284E-N287R-S322T-N388M-K394R-C430N-L432F-L434W-S436N | +++ |
| P284D-N287R-S322T-N388M-K394R-C430N-L432F-L434W-S436N | +++ |
| I283T-P284E-N287R-S322T-N388M-K394R-C430N-L432F-L434W-S436N | +++ |
| I283L-P284E-N287R-S322T-N388M-K394R-C430N-L432F-L434W-S436N | +++ |
| P284E-N285T-N287R-S322T-N388M-K394R-C430N-L432F-L434W-S436N | +++ |
| P284E-E286W-N287R-S322T-N388M-K394R-C430N-L432F-L434W-S436N | +++ |
| P284E-N287S-S322T-N388M-K394R-C430N-L432F-L434W-S436N | +++ |
| P284E-N285H-N287R-S322T-N388M-K394R-C430N-L432F-L434W-S436N | +++ |
| P284E-N287P-S322T-N388M-K394R-C430N-L432F-L434W-S436N | +++ |
| P284E-N287R-S288N-S322T-N388M-K394R-C430N-L432F-L434W-S436N | +++ |
| P284E-N287R-C321V-S322T-N388M-K394R-C430N-L432F-L434W-S436N | +++ |
| P284E-N287R-S322T-A329S-N388M-K394R-C430N-L432F-L434W-S436N | +++ |
| L6M-P284E-N287R-S322T-N388M-K394R-C430N-L432F-L434W-S436N | +++ |
| P284E-N287R-S322T-N338G-N388M-K394R-C430N-L432F-L434W-S436N | +++ |
| P284E-N287R-S322T-V379I-N388M-K394R-C430N-L432F-L434W-S436N | +++ |
| P284E-N287R-S322T-V382L-N388M-K394R-C430N-L432F-L434W-S436N | +++ |
| P284E-N287R-S322T-N388Q-K394R-C430N-L432F-L434W-S436N | +++ |
| P284E-N287R-S322T-N388M-K394R-F426V-C430N-L432F-L434W-S436N | +++ |
| P284E-N287R-S322T-N388M-K394R-F426Y-C430N-L432F-L434W-S436N | +++ |
| P284E-N287R-S322T-N388M-K394R-F426W-C430N-L432F-L434W-S436N | +++ |
| V46I-P284E-N287R-S322T-N388M-K394R-C430N-L432F-L434W-S436N | +++ |
| P284E-N287R-S322T-N388M-K394R-C430N-L432F-L434W-S436W | +++ |
| P284E-N287R-S322T-N388M-K394R-C430N-L432F-L434W-S436N-C437M | +++ |
| P284E-N287R-S322T-N388M-K394R-C430N-L432F-L434W-S436N-E441D | +++ |
| P284E-N287R-S322T-N388M-K394R-C430N-L432F-L434W-S436N-E441T | +++ |
| P284E-N287R-S322T-N388M-K394R-C430N-L432F-L434W-S436N-E441H | +++ |
| P284E-N287R-S322T-N388M-K394R-C430N-L432F-L434W-S436N-H444L | +++ |
| P284E-N287R-S322T-N388M-K394R-C430N-L432F-L434W-S436N-K447W | +++ |
| P284E-N287R-S322T-N388M-K394R-C430N-L432F-L434W-S436N-K447F | +++ |
| F183Y-N285M-N287T-S322T-N388M-K394R-C430N-L432F-L434W-S436N | +++ |
| N285M-N287T-S322T-V333M-N388M-K394R-C430N-L432F-L434W-S436N | +++ |
| I283T-N285M-N287T-S322T-N388M-K394R-C430N-L432F-L434W-S436N | +++ |
| I283V-N285M-N287T-S322T-N388M-K394R-C430N-L432F-L434W-S436N | +++ |
| P284D-N285M-N287T-S322T-N388M-K394R-C430N-L432F-L434W-S436N | +++ |
| N285P-N287T-S322T-N388M-K394R-C430N-L432F-L434W-S436N | +++ |
| N285M-N287T-Q290E-S322T-N388M-K394R-C430N-L432F-L434W-S436N | +++ |
| N285M-N287T-I292L-S322T-N388M-K394R-C430N-L432F-L434W-S436N | +++ |
| N285M-N287T-I292V-S322T-N388M-K394R-C430N-L432F-L434W-S436N | +++ |
| N51D-N285M-N287T-S322T-N388M-K394R-C430N-L432F-L434W-S436N | +++ |
| N285M-N287T-C321T-S322T-N388M-K394R-C430N-L432F-L434W-S436N | +++ |
| N285M-N287T-C321V-S322T-N388M-K394R-C430N-L432F-L434W-S436N | +++ |
| N285M-N287T-S322T-N332R-N388M-K394R-C430N-L432F-L434W-S436N | +++ |
| N285M-N287T-S322T-N332S-N388M-K394R-C430N-L432F-L434W-S436N | +++ |
| N285M-N287T-S322T-T366A-Q385W-N388M-K394R-C430N-L432F-L434W-S436N | +++ |
| N285M-N287T-S322T-Q385L-N388M-K394R-C430N-L432F-L434W-S436N | +++ |
| N285M-N287T-S322T-Q385P-N388M-K394R-C430N-L432F-L434W-S436N | +++ |
| N285M-N287T-S322T-Q385S-N388M-K394R-C430N-L432F-L434W-S436N | +++ |
| N285M-N287T-S322T-N388M-S389A-K394R-C430N-L432F-L434W-S436N | +++ |
| N285M-N287T-S322T-N388M-S389Q-K394R-C430N-L432F-L434W-S436N | +++ |
| N285M-N287T-S322T-N388M-L392Y-K394R-C430N-L432F-L434W-S436N | +++ |
| R7C-N285M-N287T-S322T-N388M-K394Q-C430N-L432F-L434W-S436N | +++ |
| N285M-N287T-S322T-N388M-K394H-C430N-L432F-L434W-S436N | +++ |
| N285M-N287T-S322T-N388M-K394L-C430N-L432F-L434W-S436N | +++ |
| N285M-N287T-S322T-N388M-K394R-F426H-C430N-L432F-L434W-S436N | +++ |
| N285M-N287T-S322T-N388M-K394R-F426I-C430N-L432F-L434W-S436N | +++ |
| N285M-N287T-S322T-N388M-K394R-F426L-C430N-L432F-L434W-S436N | +++ |
| N285M-N287T-S322T-N388M-K394R-F426T-C430N-L432F-L434W-S436N | +++ |
| N285M-N287T-S322T-N388M-K394R-F426V-C430N-L432F-L434W-S436N | +++ |
| N285M-N287T-S322T-N388M-K394R-F426W-C430N-L432F-L434W-S436N | +++ |
| N285M-N287T-S322T-N388M-K394R-C430A-L432F-L434W-S436N | +++ |
| N285M-N287T-S322T-N388M-K394R-C430N-L431P-L432F-L434W-S436N | +++ |
| N285M-N287T-S322T-N388M-K394R-C430N-L432F-L434W-S436N-C437L | +++ |
| N285M-N287T-S322T-N388M-K394R-C430N-L432F-L434W-S436N-C437T | +++ |
| N285M-N287T-S322T-N388M-K394R-C430N-L432F-L434W-S436N-C437V | +++ |
| N285M-N287T-S322T-N388M-K394R-C430N-L432F-L434W-S436N-E441G | +++ |
| N285M-N287T-S322T-N388M-K394R-C430N-L432F-L434W-S436N-E441I | +++ |
| N285M-N287T-S322T-N388M-K394R-C430N-L432F-L434W-S436N-E441L | +++ |
| N285M-N287T-S322T-N388M-K394R-C430N-L432F-L434W-S436N-E441P | +++ |
| N285M-N287T-S322T-N388M-K394R-C430N-L432F-L434W-S436N-R443H | +++ |
| N285M-N287T-S322T-N388M-K394R-C430N-L432F-L434W-S436N-H444F | +++ |
| N285M-N287T-S322T-N388M-K394R-C430N-L432F-L434W-S436N-H444I | +++ |
| N285M-N287T-S322T-N388M-K394R-C430N-L432F-L434W-S436N-H444L | +++ |
| N285M-N287T-S322T-N388M-K394R-C430N-L432F-L434W-S436N-H444M | +++ |
| N285M-N287T-S322T-N388M-K394R-C430N-L432F-L434W-S436N-H444V | +++ |
| N285M-N287T-S322T-N388M-K394R-C430N-L432F-L434W-S436N-N445A | +++ |
| N285M-N287T-S322T-N388M-K394R-C430N-L432F-L434W-S436N-N445Q | +++ |
| N285M-N287T-S322T-N388M-K394R-C430N-L432F-L434W-S436N-N445T | +++ |
| N285M-N287T-S322T-N388M-K394R-C430N-L432F-L434W-S436N-R446D | +++ |
| N285M-N287T-S322T-N388M-K394R-C430N-L432F-L434W-S436N-R446G | +++ |
| N285M-N287T-S322T-N388M-K394R-C430N-L432F-L434W-S436N-R446P | +++ |
| N285M-N287T-S322T-N388M-K394R-C430N-L432F-L434W-S436N-K449W | +++ |
| N285M-N287T-S322T-N388M-K394R-C430N-L432F-L434W-S436N-K449N | +++ |
| E72K-P284E-N287Q-S322T-N388M-K394R-C430N-L432F-L434W-S436N | +++ |
| P284E-N287Q-S322T-N388M-K394R-C430N-L432F-L434W-S436N-K447H | +++ |
| P284E-N287Q-S322T-N332S-N388M-K394R-C430N-L432F-L434W-S436N | +++ |
| P284E-N287Q-S322T-N388M-K394R-C430N-L432F-L434W-S436M | +++ |
| P284E-N287Q-S322T-N388M-K394R-C430N-L432F-L434W-S436W | +++ |
| P284E-N287Q-S322T-T336H-N388M-K394R-C430N-L432F-L434W-S436N | +++ |
| P284E-N287Q-S322T-T336N-N388M-K394R-C430N-L432F-L434W-S436N | +++ |
| P284E-N287Q-S322T-T336Q-N388M-K394R-C430N-L432F-L434W-S436N | +++ |
| S89N-P284E-N287Q-S322T-A327S-N388M-K394R-C430N-L432F-L434W-S436N | +++ |
| I283C-P284E-N287Q-S322T-N388M-K394R-C430N-L432F-L434W-S436N | +++ |
| I283H-P284E-N287Q-S322T-N388M-K394R-C430N-L432F-L434W-S436N | +++ |
| I283S-P284E-N287Q-S322T-N388M-K394R-C430N-L432F-L434W-S436N | +++ |
| I283T-P284E-N287Q-S322T-N388M-K394R-C430N-L432F-L434W-S436N | +++ |
| I283V-P284E-N287Q-S322T-N388M-K394R-C430N-L432F-L434W-S436N | +++ |
| I283V-P284D-N287Q-S322T-N388M-K394R-C430N-L432F-L434W-S436N | +++ |
| P284E-N287Q-S322T-Q331G-N388M-K394R-C430N-L432F-L434W-S436N | +++ |
| P284E-N287Q-S322T-Q385F-N388M-K394R-C430N-L432F-L434W-S436N | +++ |
| P284E-N287Q-S322T-Q385P-N388M-K394R-C430N-L432F-L434W-S436N | +++ |
| P284E-N287Q-S322T-Q385R-N388M-K394R-C430N-L432F-L434W-S436N | +++ |
| P284E-N287Q-S322T-H384S-N388M-K394R-C430N-L432F-L434W-S436N | +++ |
| P284E-N287Q-S322T-N388M-K394G-C430N-L432F-L434W-S436N | +++ |
| P284E-N287Q-S322T-N388M-C430N-L432F-L434W-S436N | +++ |
| P284E-N287Q-S322T-N388M-K394L-C430N-L432F-L434W-S436N | +++ |
| R7C-P284E-N287Q-S322T-N388M-K394R-C430N-L432F-L434W-S436N | +++ |
| L48M-P284E-N287Q-S322T-N388M-K394R-C430N-L432F-L434W-S436N | +++ |
| P284E-N287Q-S322T-N388M-K394R-C430N-L432F-L434W-S436N-E440D | +++ |
| P284E-N287Q-S322T-N388M-K394R-C430N-L432F-L434W-S436N-E440I | +++ |
| P284E-N287Q-S322T-N388M-K394R-C430N-L432F-L434W-S436N-E441L | +++ |
| P284E-N287Q-S322T-N388M-K394R-C430N-L432F-L434W-S436N-E441T | +++ |
| P284E-N287Q-S322T-N388M-K394R-E428M-C430N-L432F-L434W-S436N | +++ |
| P284E-N287Q-S322T-N388M-K394R-E428T-C430N-L432F-L434W-S436N | +++ |
| P284E-N287Q-S322T-A327S-N388M-K394R-C430N-L432F-L434W-S436N | +++ |
| S189C-P284E-N287Q-S322T-N388M-K394R-C430N-L432F-L434W-S436N | +++ |
| P284E-N287Q-C321V-S322T-N388M-K394R-C430N-L432F-L434W-S436N | +++ |
| P284E-N287Q-S322T-N388M-K394R-F426L-C430N-L432F-L434W-S436N | +++ |
| P284E-N287Q-S322T-N388M-K394R-F426Y-C430N-L432F-L434W-S436N | +++ |
| N285M-N287T-Q290E-S322T-N388M-K394R-C430A-L432F-L434W-S436N | +++ |
| I283T-N285M-N287T-Q290E-S322T-N388M-K394R-C430A-L432F-L434W-S436N | +++ |
| N285P-N287T-Q290E-S322T-N388M-K394R-C430A-L432F-L434W-S436N | +++ |
| I283T-N285P-N287T-Q290E-S322T-N388M-K394R-C430A-L432F-L434W-S436N | +++ |
| P284D-N285P-N287T-Q290E-S322T-N388M-K394R-C430A-L432F-L434W-S436N | +++ |
| I283T-N285P-N287T-S322T-N388M-K394R-C430N-L432F-L434W-S436N | +++ |
| I283T-N285M-N287T-Q290E-S322T-N388M-K394R-C430N-L432F-L434W-S436N | +++ |
| I283T-N285M-N287T-S322T-N388M-K394R-C430A-L432F-L434W-S436N | +++ |
| P284D-N285P-N287T-S322T-N388M-K394R-C430N-L432F-L434W-S436N | +++ |
| I283T-N285P-N287T-S322T-N388M-K394R-C430A-L432F-L434W-S436N | +++ |
| P284D-N285P-N287T-Q290E-S322T-N388M-K394R-C430N-L432F-L434W-S436N | +++ |
| P284D-N285P-N287T-S322T-N388M-K394R-C430A-L432F-L434W-S436N | +++ |
| I283T-P284D-N285M-N287T-S322T-N388M-K394R-C430N-L432F-L434W-S436N | +++ |
| I283T-P284D-N285P-N287T-S322T-N388M-K394R-C430N-L432F-L434W-S436N | +++ |
| I283T-P284D-N285M-N287T-Q290E-S322T-N388M-K394R-C430N-L432F-L434W-S436N | +++ |
| I283T-P284D-N285M-N287T-S322T-N388M-K394R-C430A-L432F-L434W-S436N | +++ |
| I283T-P284D-N285P-N287T-Q290E-S322T-N388M-K394R-C430N-L432F-L434W-S436N | +++ |
| I283T-P284D-N285P-N287T-S322T-N388M-K394R-C430A-L432F-L434W-S436N | +++ |
| I283T-P284D-N285M-N287T-Q290E-S322T-N388M-K394R-C430A-L432F-L434W-S436N | +++ |
| I283T-P284D-N285P-N287T-Q290E-S322T-N388M-K394R-C430A-L432F-L434W-S436N | +++ |
| P284D-N285M-N287T-Q290E-S322T-N388M-K394R-C430N-L432F-L434W-S436N | +++ |
| P284D-N285M-N287T-S322T-N388M-K394R-C430A-L432F-L434W-S436N | +++ |
| N285P-N287T-Q290E-S322T-N388M-K394R-C430N-L432F-L434W-S436N | +++ |
| N285P-N287T-S322T-N388M-K394R-C430A-L432F-L434W-S436N | +++ |
| I283T-N285P-N287T-Q290E-S322T-N388M-K394R-C430N-L432F-L434W-S436N | +++ |
| I283T-P284D-N287R-S322T-N388M-K394R-C430N-L432F-L434W-S436N | +++ |
| I283T-P284D-N287P-S322T-N388M-K394R-C430N-L432F-L434W-S436N | +++ |
| I283T-P284D-N287R-S322T-N388M-C430N-L432F-L434W-S436N | +++ |
| I283T-P284D-E286W-N287P-S322T-N388M-K394R-C430N-L432F-L434W-S436N | +++ |
| I283T-P284D-N287P-S322T-N388M-C430N-L432F-L434W-S436N | +++ |
| I283C-P284E-E286W-N287P-S322T-N388M-K394R-C430N-L432F-L434W-S436N | +++ |
| I283T-P284E-E286W-N287P-S322T-N388M-K394R-C430N-L432F-L434W-S436N | +++ |
| I283C-P284E-E286W-N287P-S322T-N388M-C430N-L432F-L434W-S436N | +++ |
| I283T-P284E-E286W-N287P-S322T-N388M-C430N-L432F-L434W-S436N | +++ |
| I283T-P284D-E286W-N287P-S322T-N388M-C430N-L432F-L434W-S436N | +++ |
| I283C-P284E-N287R-S322T-N388M-K394R-C430N-L432F-L434W-S436N | +++ |
| I283C-P284D-N287P-S322T-N388M-K394R-C430N-L432F-L434W-S436N | +++ |
| I283C-P284D-N287R-S322T-N388M-C430N-L432F-L434W-S436N | +++ |
| I283C-P284D-N287P-S322T-N388M-C430N-L432F-L434W-S436N | +++ |
| I283C-P284E-N287R-S322T-N388M-C430N-L432F-L434W-S436N | +++ |
| I283T-P284E-N287R-S322T-N388M-C430N-L432F-L434W-S436N | +++ |
| P284D-N287R-S322T-N388M-C430N-L432F-L434W-S436N | +++ |
| I283C-P284E-E286W-N287R-S322T-N388M-C430N-L432F-L434W-S436N | +++ |
| I283C-P284E-N287P-S322T-N388M-C430N-L432F-L434W-S436N | +++ |
| I283T-P284E-N287P-S322T-N388M-C430N-L432F-L434W-S436N | +++ |
| I283T-P284E-E286W-N287R-S322T-N388M-K394R-C430N-L432F-L434W-S436N | +++ |
| I283T-P284E-N287P-S322T-N388M-K394R-C430N-L432F-L434W-S436N | +++ |
| P284E-E286W-N287R-S322T-N388M-C430N-L432F-L434W-S436N | +++ |
| P284E-N287P-S322T-N388M-C430N-L432F-L434W-S436N | +++ |
| I283T-P284E-E286W-N287R-S322T-N388M-C430N-L432F-L434W-S436N | +++ |
| P284D-N287P-S322T-N388M-C430N-L432F-L434W-S436N | +++ |
| I283C-P284E-E286W-N287R-S322T-N388M-K394R-C430N-L432F-L434W-S436N | +++ |
| I283C-P284E-N287P-S322T-N388M-K394R-C430N-L432F-L434W-S436N | +++ |
| P284D-N287P-S322T-N388M-K394R-C430N-L432F-L434W-S436N | +++ |
| I283C-P284D-N287Q-S322T-N388M-K394R-C430N-L432F-L434W-S436N | +++ |
| I283C-P284E-N287Q-S322T-N388M-C430N-L432F-L434W-S436N | +++ |
| I283C-P284D-N287Q-S322T-N388M-C430N-L432F-L434W-S436N | +++ |
| I283C-P284E-E286W-N287Q-S322T-N388M-C430N-L432F-L434W-S436N | +++ |
| I283T-P284D-N287Q-S322T-N388M-K394R-C430N-L432F-L434W-S436N | +++ |
| I283T-P284E-E286W-N287Q-S322T-N388M-K394R-C430N-L432F-L434W-S436N | +++ |
| I283T-P284E-N287Q-S322T-N388M-C430N-L432F-L434W-S436N | +++ |
| I283T-P284D-N287Q-S322T-N388M-C430N-L432F-L434W-S436N | +++ |
| I283T-P284E-E286W-N287Q-S322T-N388M-C430N-L432F-L434W-S436N | +++ |
| P284D-N287Q-S322T-N388M-C430N-L432F-L434W-S436N | +++ |
| P284E-E286W-N287Q-S322T-N388M-C430N-L432F-L434W-S436N | +++ |
| N285M-N287T-S322T-N388M-K394R-C430N-L432F-L434W-S436N-P458E | +++ |
| N285M-N287T-S322T-N388M-K394R-C430N-L432F-L434W-S436N-G462V | +++ |
| N285M-N287T-S322T-N388M-K394R-C430N-L432F-L434W-S436N-E459P | +++ |
| N285M-N287T-S322T-N388M-K394R-C430N-L432F-L434W-S436N-E459R | +++ |
| N285M-N287T-S322T-N388M-K394R-C430N-L432F-L434W-S436N-H461A | +++ |
| N285M-N287T-S322T-N388M-K394R-C430N-L432F-L434W-S436N-H461D | +++ |
| N285M-N287T-S322T-N388M-K394R-C430N-L432F-L434W-S436N-H461E | +++ |
| N285M-N287T-S322T-N388M-K394R-C430N-L432F-L434W-S436N-H461G | +++ |
| N285M-N287T-S322T-N388M-K394R-C430N-L432F-L434W-S436N-H461N | +++ |
| P45Q-N285M-N287T-S322T-N388M-K394R-C430N-L432F-L434W-S436N-H461S | +++ |
| N285M-N287T-S322T-N388M-K394R-C430N-L432F-L434W-S436N-G462A | +++ |
| N285M-N287T-S322T-N388M-K394R-C430N-L432F-L434W-S436N-G462C | +++ |
| N285M-N287T-S322T-N388M-K394R-C430N-L432F-L434W-S436N-G462P | +++ |
| N285M-N287T-S322T-N388M-K394R-C430N-L432F-L434W-S436N-G462I | +++ |
| N285M-N287T-S322T-N388M-K394R-C430N-L432F-L434W-S436N-A463L | +++ |
| N285M-N287T-S322T-N388M-K394R-C430N-L432F-L434W-S436N-A463P | +++ |
| N285M-N287T-S322T-N388M-K394R-C430N-L432F-L434W-S436N-Y469D | +++ |
| N285M-N287T-S322T-N388M-K394R-C430N-L432F-L434W-S436N-Y469E | +++ |
| N285M-N287T-S322T-N388M-K394R-C430N-L432F-L434W-S436N-Y469S | +++ |
| N285M-N287T-S322T-N388M-K394R-C430N-L432F-L434W-S436N-Y469T | +++ |
| N285M-N287T-S322T-N388M-K394R-C430N-L432F-L434W-S436N-Y471F | +++ |
| H141L-N285M-N287T-S322T-N388M-K394R-C430N-L432F-L434W-S436N | +++ |
| N144S-N285M-N287T-S322T-N388M-K394R-C430N-L432F-L434W-S436N | +++ |
| R15H-N285M-N287T-S322T-N388M-K394R-C430N-L432F-L434W-S436N | +++ |
| P284E-N287Q-S322T-N388M-K394R-C430N-L432F-L434W-S436N-E459L | +++ |
| V46I-P284E-N287Q-S322T-N388M-K394R-C430N-L432F-L434W-S436N-N460V | +++ |
| P284E-N287Q-S322T-N388M-K394R-C430N-L432F-L434W-S436N-N460G | +++ |
| P284E-N287Q-S322T-N388M-K394R-C430N-L432F-L434W-S436N-N460I | +++ |
| P284E-N287Q-S322T-N388M-K394R-C430N-L432F-L434W-S436N-H461A | +++ |
| P284E-N287Q-S322T-N388M-K394R-C430N-L432F-L434W-S436N-H461G | +++ |
| P284E-N287Q-S322T-N388M-K394R-C430N-L432F-L434W-S436N-H461N | +++ |
| P284E-N287Q-S322T-N388M-K394R-C430N-L432F-L434W-S436N-H461T | +++ |
| P284E-N287Q-S322T-N388M-K394R-C430N-L432F-L434W-S436N-H461D | +++ |
| P284E-N287Q-S322T-N388M-K394R-C430N-L432F-L434W-S436N-A463E | +++ |
| P284E-N287Q-S322T-N388M-K394R-C430N-L432F-L434W-S436N-A463G | +++ |
| P284E-N287Q-S322T-N388M-K394R-C430N-L432F-L434W-S436N-F467W | +++ |
| P284E-N287Q-S322T-N388M-K394R-C430N-L432F-L434W-S436N-Y469D | +++ |
| P284E-N287Q-S322T-N388M-K394R-C430N-L432F-L434W-S436N-Y469N | +++ |
| P284E-N287Q-S322T-N388M-K394R-C430N-L432F-L434W-S436N-Y469S | +++ |
| P284E-N287Q-S322T-N388M-K394R-C430N-L432F-L434W-S436N-Y469T | +++ |
| P284E-N287Q-S322T-N388M-K394R-C430N-L432F-L434W-S436N-A470G | +++ |
| P284E-N287Q-S322T-N388M-K394R-C430N-L432F-L434W-S436N-Y471F | +++ |
| H141Q-P284E-N287Q-S322T-N388M-K394R-C430N-L432F-L434W-S436N | +++ |
| H141S-P284E-N287Q-S322T-N388M-K394R-C430N-L432F-L434W-S436N | +++ |
| H141Y-P284E-N287Q-S322T-N388M-K394R-C430N-L432F-L434W-S436N | +++ |
| N285M-N287T-S322T-N388M-K394R-C430N-L432F-L434W-S436N-dP458 | +++ |
| N285M-N287T-S322T-N388M-K394R-C430N-L432F-L434W-S436N-dE459 | +++ |
| N285M-N287T-S322T-N388M-K394R-C430N-L432F-L434W-S436N-dN460 | +++ |
| N285M-N287T-S322T-N388M-K394R-C430N-L432F-L434W-S436N-dH461 | +++ |
| N285M-N287T-S322T-N388M-K394R-C430N-L432F-L434W-S436N-dG462 | +++ |
| N285M-N287T-S322T-N388M-K394R-C430N-L432F-L434W-S436N-dA463 | +++ |
| P284E-N287Q-S322T-N388M-K394R-C430N-L432F-L434W-S436N-dP458 | +++ |
| P284E-N287Q-S322T-N388M-K394R-C430N-L432F-L434W-S436N-dE459 | +++ |
| P284E-N287Q-S322T-N388M-K394R-C430N-L432F-L434W-S436N-dN460 | +++ |
| P284E-N287Q-S322T-N388M-K394R-C430N-L432F-L434W-S436N-dH461 | +++ |
| P284E-N287Q-S322T-N388M-K394R-C430N-L432F-L434W-S436N-dG462 | +++ |
| P284E-N287Q-S322T-N388M-K394R-C430N-L432F-L434W-S436N-dA463 | +++ |
| P284E-N287Q-S322T-N388M-K394R-C430N-L432F-L434W-S436N-H461G-Y469D | +++ |
| P284E-N287Q-S322T-N388M-K394R-C430N-L432F-L434W-S436N-A463G-Y469D | +++ |
| P284E-N287Q-S322T-N388M-K394R-C430N-L432F-L434W-S436N-H461G-A463G-Y469D | +++ |
| P284E-N287Q-S322T-N388M-K394R-C430N-L432F-L434W-S436N-N460G-H461G-A463G-Y469D | +++ |
| P284D-N287P-S322T-N388M-C430N-L432F-L434W-S436N-N460G | +++ |
| P284D-N287P-S322T-N388M-C430N-L432F-L434W-S436N-N460I | +++ |
| P284D-N287P-S322T-N388M-C430N-L432F-L434W-S436N-H461G | +++ |
| P284D-N287P-S322T-N388M-C430N-L432F-L434W-S436N-A463G | +++ |
| P284D-N287P-S322T-N388M-C430N-L432F-L434W-S436N-Y469D | +++ |
| P284D-N287P-S322T-N388M-C430N-L432F-L434W-S436N-N460G-H461G | +++ |
| P284D-N287P-S322T-N388M-C430N-L432F-L434W-S436N-N460G-A463G | +++ |
| P284D-N287P-S322T-N388M-C430N-L432F-L434W-S436N-N460G-Y469D | +++ |
| P284D-N287P-S322T-N388M-C430N-L432F-L434W-S436N-N460I-A463G | +++ |
| P284D-N287P-S322T-N388M-C430N-L432F-L434W-S436N-N460I-Y469D | +++ |
| P284D-N287P-S322T-N388M-C430N-L432F-L434W-S436N-H461G-A463G | +++ |
| P284D-N287P-S322T-N388M-C430N-L432F-L434W-S436N-H461G-Y469D | +++ |
| P284D-N287P-S322T-N388M-C430N-L432F-L434W-S436N-A463G-Y469D | +++ |
| P284D-N287P-S322T-N388M-C430N-L432F-L434W-S436N-N460G-H461G-A463G | +++ |
| P284D-N287P-S322T-N388M-C430N-L432F-L434W-S436N-N460G-H461G-Y469D | +++ |
| P284D-N287P-S322T-N388M-C430N-L432F-L434W-S436N-N460G-A463G-Y469D | +++ |
| P284D-N287P-S322T-N388M-C430N-L432F-L434W-S436N-N460I-H461G-Y469D | +++ |
| P284D-N287P-S322T-N388M-C430N-L432F-L434W-S436N-N460I-A463G-Y469D | +++ |
| P284D-N287P-S322T-N388M-C430N-L432F-L434W-S436N-H461G-A463G-Y469D | +++ |
| P284D-N287P-S322T-N388M-C430N-L432F-L434W-S436N-N460G-H461G-A463G-Y469D | +++ |
| P284D-N287P-S322T-N388M-C430N-L432F-L434W-S436N-N460I-H461G-A463G-Y469D | +++ |
| I283T-P284D-N287P-S322T-N388M-C430N-L432F-L434W-S436N-N460G | +++ |
| I283T-P284D-N287P-S322T-N388M-C430N-L432F-L434W-S436N-A463G | +++ |
| I283T-P284D-N287P-S322T-N388M-C430N-L432F-L434W-S436N-Y469D | +++ |
| I283T-P284D-N287P-S322T-N388M-C430N-L432F-L434W-S436N-N460G-A463G | +++ |
| I283T-P284D-N287P-S322T-N388M-C430N-L432F-L434W-S436N-N460G-Y469D | +++ |
| I283T-P284D-N287P-S322T-N388M-C430N-L432F-L434W-S436N-N460G-A463G-Y469D | +++ |
| I283T-P284D-N287P-S322T-N388M-C430N-L432F-L434W-S436N-N460I-H461G-Y469D | +++ |
| I283T-P284D-N287P-S322T-N388M-C430N-L432F-L434W-S436N-N460I-A463G-Y469D | +++ |
| I283T-P284D-N287P-S322T-N388M-C430N-L432F-L434W-S436N-H461G-A463G-Y469D | +++ |
| I283T-P284D-N287P-S322T-N388M-C430N-L432F-L434W-S436N-N460G-H461G-A463G-Y469D | +++ |
| I283T-P284D-N287P-S322T-N388M-C430N-L432F-L434W-S436N-N460I-H461G | +++ |
| I283T-P284D-N287P-S322T-N388M-C430N-L432F-L434W-S436N-N460I-A463G | +++ |
| I283T-P284D-N287P-S322T-N388M-C430N-L432F-L434W-S436N-N460I-Y469D | +++ |
| I283T-P284D-N287P-S322T-N388M-C430N-L432F-L434W-S436N-H461G-A463G | +++ |
| I283T-P284D-N287P-S322T-N388M-C430N-L432F-L434W-S436N-H461G-Y469D | +++ |
| I283T-P284D-N287P-S322T-N388M-C430N-L432F-L434W-S436N-A463G-Y469D | +++ |
| I283T-P284D-N287P-S322T-N388M-C430N-L432F-L434W-S436N-N460G-H461G-A463G | +++ |
| I283T-P284D-N287P-S322T-N388M-C430N-L432F-L434W-S436N-N460G-H461G-Y469D | +++ |
| I283T-P284D-N287Q-S322T-N388M-C430N-L432F-L434W-S436N-H461G | +++ |
| I283T-P284D-N287Q-S322T-N388M-C430N-L432F-L434W-S436N-A463G | +++ |
| I283T-P284D-N287Q-S322T-N388M-C430N-L432F-L434W-S436N-Y469D | +++ |
| I283T-P284D-N287Q-S322T-N388M-C430N-L432F-L434W-S436N-N460G-H461G | +++ |
| I283T-P284D-N287Q-S322T-N388M-C430N-L432F-L434W-S436N-N460G-A463G | +++ |
| I283T-P284D-N287Q-S322T-N388M-C430N-L432F-L434W-S436N-N460G-Y469D | +++ |
| I283T-P284D-N287Q-S322T-N388M-C430N-L432F-L434W-S436N-N460I-Y469D | +++ |
| I283T-P284D-N287Q-S322T-N388M-C430N-L432F-L434W-S436N-H461G-Y469D | +++ |
| I283T-P284D-N287Q-S322T-N388M-C430N-L432F-L434W-S436N-A463G-Y469D | +++ |
| I283T-P284D-N287Q-S322T-N388M-C430N-L432F-L434W-S436N-N460G-H461G-A463G | +++ |
| I283T-P284D-N287Q-S322T-N388M-C430N-L432F-L434W-S436N-N460G-H461G-Y469D | +++ |
| I283T-P284D-N287Q-S322T-N388M-C430N-L432F-L434W-S436N-N460I-H461G-Y469D | +++ |
| I283T-P284D-N287Q-S322T-N388M-C430N-L432F-L434W-S436N-N460G-H461G-A463G-Y469D | +++ |
| P284D-N287P-S322T-N388M-C430N-L432F-L434W-S436N-dG462 | +++ |
| I283T-P284D-N287P-S322T-N388M-C430N-L432F-L434W-S436N-dG462 | +++ |
| P284D-N287P-S322T-N388M-C430N-L432F-L434W-S436N-dA463 | +++ |
| I283T-P284D-N287P-S322T-N388M-C430N-L432F-L434W-S436N-dA463 | +++ |
| I283T-P284D-N287Q-S322T-N388M-C430N-L432F-L434W-S436N-dG462 | +++ |
| I283T-P284D-N287Q-S322T-N388M-C430N-L432F-L434W-S436N-dA463 | +++ |
| I283T-P284D-N285P-N287T-S322T-N388M-K394R-C430N-L432F-L434W-S436N-G462A | +++ |
| I283T-P284D-N285P-N287T-S322T-N388M-K394R-C430N-L432F-L434W-S436N-G462P | +++ |
| I283T-P284D-N285P-N287T-S322T-N388M-K394R-C430N-L432F-L434W-S436N-G462V | +++ |
| I283T-P284D-N285P-N287T-S322T-N388M-K394R-C430N-L432F-L434W-S436N-Y469D | +++ |
| I283T-P284D-N285P-N287T-S322T-N388M-K394R-C430N-L432F-L434W-S436N-G462A-A463P | +++ |
| I283T-P284D-N285P-N287T-S322T-N388M-K394R-C430N-L432F-L434W-S436N-G462A-Y469D | +++ |
| I283T-P284D-N285P-N287T-S322T-N388M-K394R-C430N-L432F-L434W-S436N-G462P-A463P | +++ |
| I283T-P284D-N285P-N287T-S322T-N388M-K394R-C430N-L432F-L434W-S436N-G462P-Y469D | +++ |
| N285M-N287T-S322T-N388M-K394R-C430N-L432F-L434W-S436N-G462V-A463P | +++ |
| N285M-N287T-S322T-N388M-K394R-C430N-L432F-L434W-S436N-G462V-Y469D | +++ |
| N285M-N287T-S322T-N388M-K394R-C430N-L432F-L434W-S436N-A463P-Y469D | +++ |
| N285M-N287T-S322T-N388M-K394R-C430N-L432F-L434W-S436N-G462A-A463P-Y469D | +++ |
| N285M-N287T-S322T-N388M-K394R-C430N-L432F-L434W-S436N-G462P-A463P-Y469D | +++ |
| I283T-P284D-N285P-N287T-S322T-N388M-K394R-C430N-L432F-L434W-S436N-G462V-A463P | +++ |
| I283T-P284D-N285P-N287T-S322T-N388M-K394R-C430N-L432F-L434W-S436N-G462V-Y469D | +++ |
| I283T-P284D-N285P-N287T-S322T-N388M-K394R-C430N-L432F-L434W-S436N-A463P-Y469D | +++ |
| I283T-P284D-N285P-N287T-S322T-N388M-K394R-C430N-L432F-L434W-S436N-G462A-A463P-Y469D | +++ |
| I283T-P284D-N285P-N287T-S322T-N388M-K394R-C430N-L432F-L434W-S436N-G462P-A463P-Y469D | +++ |
| I283T-P284D-N285P-N287T-S322T-N388M-K394R-C430N-L432F-L434W-S436N-G462V-A463P-Y469D | +++ |
| I283T-P284D-N285M-N287T-S322T-N388M-K394R-C430A-L432F-L434W-S436N-G462A | +++ |
| I283T-P284D-N285M-N287T-S322T-N388M-K394R-C430A-L432F-L434W-S436N-G462P | +++ |
| I283T-P284D-N285M-N287T-S322T-N388M-K394R-C430A-L432F-L434W-S436N-G462V | +++ |
| I283T-P284D-N285M-N287T-S322T-N388M-K394R-C430A-L432F-L434W-S436N-Y469D | +++ |
| I283T-P284D-N285M-N287T-S322T-N388M-K394R-C430A-L432F-L434W-S436N-G462A-A463P | +++ |
| I283T-P284D-N285M-N287T-S322T-N388M-K394R-C430A-L432F-L434W-S436N-G462A-Y469D | +++ |
| I283T-P284D-N285M-N287T-S322T-N388M-K394R-C430A-L432F-L434W-S436N-G462P-A463P | +++ |
| I283T-P284D-N285M-N287T-S322T-N388M-K394R-C430A-L432F-L434W-S436N-G462P-Y469D | +++ |
| I283T-P284D-N285M-N287T-S322T-N388M-K394R-C430A-L432F-L434W-S436N-G462V-A463P | +++ |
| I283T-P284D-N285M-N287T-S322T-N388M-K394R-C430A-L432F-L434W-S436N-G462V-Y469D | +++ |
| I283T-P284D-N285M-N287T-S322T-N388M-K394R-C430A-L432F-L434W-S436N-A463P-Y469D | +++ |
| I283T-P284D-N285M-N287T-S322T-N388M-K394R-C430A-L432F-L434W-S436N-G462A-A463P-Y469D | +++ |
| I283T-P284D-N285M-N287T-S322T-N388M-K394R-C430A-L432F-L434W-S436N-G462P-A463P-Y469D | +++ |
| I283T-P284D-N285M-N287T-S322T-N388M-K394R-C430A-L432F-L434W-S436N-G462V-A463P-Y469D | +++ |
| I283T-N285M-N287T-Q290E-S322T-N388M-K394R-C430A-L432F-L434W-S436N-G462A | +++ |
| I283T-N285M-N287T-Q290E-S322T-N388M-K394R-C430A-L432F-L434W-S436N-G462P | +++ |
| I283T-N285M-N287T-Q290E-S322T-N388M-K394R-C430A-L432F-L434W-S436N-G462V | +++ |
| I283T-N285M-N287T-Q290E-S322T-N388M-K394R-C430A-L432F-L434W-S436N-A463P | +++ |
| I283T-N285M-N287T-Q290E-S322T-N388M-K394R-C430A-L432F-L434W-S436N-Y469D | +++ |
| I283T-N285M-N287T-Q290E-S322T-N388M-K394R-C430A-L432F-L434W-S436N-G462A-A463P | +++ |
| I283T-N285M-N287T-Q290E-S322T-N388M-K394R-C430A-L432F-L434W-S436N-G462A-Y469D | +++ |
| I283T-N285M-N287T-Q290E-S322T-N388M-K394R-C430A-L432F-L434W-S436N-G462P-A463P | +++ |
| I283T-N285M-N287T-Q290E-S322T-N388M-K394R-C430A-L432F-L434W-S436N-G462P-Y469D | +++ |
| I283T-N285M-N287T-Q290E-S322T-N388M-K394R-C430A-L432F-L434W-S436N-G462V-A463P | +++ |
| I283T-N285M-N287T-Q290E-S322T-N388M-K394R-C430A-L432F-L434W-S436N-G462V-Y469D | +++ |
| I283T-N285M-N287T-Q290E-S322T-N388M-K394R-C430A-L432F-L434W-S436N-A463P-Y469D | +++ |
| I283T-N285M-N287T-Q290E-S322T-N388M-K394R-C430A-L432F-L434W-S436N-G462A-A463P-Y469D | +++ |
| I283T-N285M-N287T-Q290E-S322T-N388M-K394R-C430A-L432F-L434W-S436N-G462P-A463P-Y469D | +++ |
| I283T-N285M-N287T-Q290E-S322T-N388M-K394R-C430A-L432F-L434W-S436N-G462V-A463P-Y469D | +++ |
| I283T-N285P-N287T-Q290E-S322T-N388M-K394R-C430A-L432F-L434W-S436N-G462A | +++ |
| I283T-N285P-N287T-Q290E-S322T-N388M-K394R-C430A-L432F-L434W-S436N-G462P | +++ |
| I283T-N285P-N287T-Q290E-S322T-N388M-K394R-C430A-L432F-L434W-S436N-G462V | +++ |
| I283T-N285P-N287T-Q290E-S322T-N388M-K394R-C430A-L432F-L434W-S436N-A463P | +++ |
| I283T-N285P-N287T-Q290E-S322T-N388M-K394R-C430A-L432F-L434W-S436N-Y469D | +++ |
| I283T-N285P-N287T-Q290E-S322T-N388M-K394R-C430A-L432F-L434W-S436N-G462A-A463P | +++ |
| I283T-N285P-N287T-Q290E-S322T-N388M-K394R-C430A-L432F-L434W-S436N-G462A-Y469D | +++ |
| I283T-N285P-N287T-Q290E-S322T-N388M-K394R-C430A-L432F-L434W-S436N-G462P-A463P | +++ |
| I283T-N285P-N287T-Q290E-S322T-N388M-K394R-C430A-L432F-L434W-S436N-G462P-Y469D | +++ |
| I283T-N285P-N287T-Q290E-S322T-N388M-K394R-C430A-L432F-L434W-S436N-G462V-A463P | +++ |
| I283T-N285P-N287T-Q290E-S322T-N388M-K394R-C430A-L432F-L434W-S436N-G462V-Y469D | +++ |
| I283T-N285P-N287T-Q290E-S322T-N388M-K394R-C430A-L432F-L434W-S436N-A463P-Y469D | +++ |
| I283T-N285P-N287T-Q290E-S322T-N388M-K394R-C430A-L432F-L434W-S436N-G462A-A463P-Y469D | +++ |
| I283T-N285P-N287T-Q290E-S322T-N388M-K394R-C430A-L432F-L434W-S436N-G462P-A463P-Y469D | +++ |
| I283T-N285P-N287T-Q290E-S322T-N388M-K394R-C430A-L432F-L434W-S436N-G462V-A463P-Y469D | +++ |
| N285M-N287T-S322T-N388M-K394R-C430N-L432F-L434W-S436N-G462A-A463P | +++ |
| N285M-N287T-S322T-N388M-K394R-C430N-L432F-L434W-S436N-G462A-Y469D | +++ |
| N285M-N287T-S322T-N388M-K394R-C430N-L432F-L434W-S436N-G462P-A463P | +++ |
| N285M-N287T-S322T-N388M-K394R-C430N-L432F-L434W-S436N-G462P-Y469D | +++ |
| R7C-I283T-N285P-N287T-Q290E-S322T-Q385D-N388M-K394R-C430A-L432F-L434W-S436N-dG462-dA463 | +++ |
| I283T-N285P-N287T-Q290E-S322T-N388M-K394R-i421aP-C430A-L432F-L434W-S436N-dG462-dA463 | +++ |
| P184A-I283T-N285P-N287T-Q290E-S322T-N388M-K394R-C430A-L432F-L434W-S436N-dG462-dA463 | +++ |
| I283T-N285P-N287T-Q290E-S322T-N388M-K394C-C430A-L432F-L434W-S436N-dG462-dA463 | +++ |
| I283T-N285P-N287T-Q290E-S322T-N388M-K394R-R422S-C430A-L432F-L434W-S436N-dG462-dA463 | +++ |
| I283T-N285P-N287T-Q290E-S322T-N388M-K394R-H419P-C430A-L432F-L434W-S436N-dG462-dA463 | +++ |
| I283T-N285P-N287T-Q290E-S322T-N388M-K394R-C430A-L432F-L434W-S436N-N460L-dG462-dA463 | +++ |
| I283T-N285P-N287T-Q290E-S322T-N388M-K394R-C430A-L432F-L434W-S436N-S439L-dG462-dA463 | +++ |
| I283T-N285P-N287T-Q290E-S322T-N388M-K394R-C430A-L432F-L434W-S436N-P458L-dG462-dA463 | +++ |
| I283T-N285P-N287T-Q290E-S322T-N388M-K394R-C430A-L432F-L434W-S436N-L457A-dG462-dA463 | +++ |
| I283T-N285P-N287T-Q290E-S322T-N388M-K394R-C430A-L432F-L434W-S436N-P458C-dG462-dA463 | +++ |
| I283T-N285P-N287T-Q290E-S322T-N388M-K394R-C430A-L432F-L434W-S436N-E459T-dG462-dA463 | +++ |
| I283T-N285P-N287T-Q290E-S322T-N388M-K394R-C430A-L432F-L434W-S436N-P458M-dG462-dA463 | +++ |
| I283T-N285P-N287T-Q290E-S322T-N388M-K394R-C430A-L432F-L434W-S436N-P458I-dG462-dA463 | +++ |
| I283T-N285P-N287T-Q290E-S322T-N388M-K394R-C430A-L432F-L434W-S436N-E459G-dG462-dA463 | +++ |
| I283T-N285P-N287T-Q290E-S322T-N388M-K394R-C430A-L432F-L434W-S436N-P458R-dG462-dA463 | +++ |
| I283T-N285P-N287T-Q290E-S322T-N388M-K394R-C430A-L432F-L434W-S436N-P458Q-dG462-dA463 | +++ |
| I283T-N285P-N287T-Q290E-S322T-N388M-K394R-C430A-L432F-L434W-S436N-N460H-dG462-dA463 | +++ |
| I283T-N285P-N287T-Q290E-S322T-N388M-K394R-C430A-L432F-L434W-S436N-N460Y-dG462-dA463 | +++ |
| I283T-N285P-N287T-Q290E-S322T-N388M-K394R-C430A-L432F-L434W-S436N-N460I-dG462-dA463 | +++ |
| I283T-N285P-N287T-Q290E-S322T-N388M-K394R-C430A-L432F-L434W-S436N-N460M-dG462-dA463 | +++ |
| I283T-P284D-N287P-S322T-N388M-K423C-C430N-L432F-L434W-S436N-A463G-Y469D | ++++ |
| I283T-P284D-N285A-N287P-S322T-N388M-C430N-L432F-L434W-S436N-A463G-Y469D | ++++ |
| I283T-P284D-N285I-N287P-S322T-N388M-C430N-L432F-L434W-S436N-A463G-Y469D | ++++ |
| I283T-P284D-N285L-N287P-S322T-N388M-C430N-L432F-L434W-S436N-A463G-Y469D | ++++ |
| I283T-P284D-N287P-S322T-N388M-C430N-L432F-L434W-S436T-A463G-Y469D | ++++ |
| I283F-P284D-N287P-S322T-N388M-C430N-L432F-L434W-S436N-A463G-Y469D | ++++ |
| I283S-P284D-N287P-S322T-N388M-C430N-L432F-L434W-S436N-A463G-Y469D | ++++ |
| I283T-P284D-N287P-S288T-S322T-N388M-C430N-L432F-L434W-S436N-A463G-Y469D | ++++ |
| I283T-P284D-N287P-S322T-N388M-S429A-C430N-L432F-L434W-S436N-A463G-Y469D | ++++ |
| I283T-P284D-N287P-S322T-H384A-N388M-C430N-L432F-L434W-S436N-A463G-Y469D | ++++ |
| I283T-P284D-N287P-S322T-H384T-N388M-C430N-L432F-L434W-S436N-A463G-Y469D | ++++ |
| I283T-P284D-N287P-S322T-N388M-C430N-L432F-L434W-S436N-H444R-A463G-Y469D | ++++ |
| I283T-P284D-N287P-S322T-N388M-P393A-C430N-L432F-L434W-S436N-A463G-Y469D | ++++ |
| I283T-P284D-N287P-S322T-N388M-R422F-C430N-L432F-L434W-S436N-A463G-Y469D | ++++ |
| I283T-P284D-N287P-S322T-N388M-R422Q-C430N-L432F-L434W-S436N-A463G-Y469D | ++++ |
| I283T-P284D-N287P-S322T-N388M-R422V-C430N-L432F-L434W-S436N-A463G-Y469D | ++++ |
| I283T-P284D-N287P-S322T-N388M-E421T-C430N-L432F-L434W-S436N-A463G-Y469D | ++++ |
| I283T-P284D-N287P-S322T-N388M-E421V-C430N-L432F-L434W-S436N-A463G-Y469D | ++++ |
| I283T-P284D-N287P-S322T-N388M-C430N-L432F-L434W-S436N-E459P-A463G-Y469D | ++++ |
| I283T-P284D-N287P-S322T-N388M-C430N-L432F-L434W-S436N-A463G-Y469T | ++++ |
| I283T-P284D-E286C-N287P-S322T-N388M-C430N-L432F-L434W-S436N-A463G-Y469D | ++++ |
| I283T-P284D-N287P-S322T-N388M-C430N-L432F-L434W-S436N-A463F-Y469D | ++++ |
| I283T-P284D-N287P-S322T-N388M-C430N-L432F-L434W-S436N-G462P-A463G-Y469D | ++++ |
| I283T-P284D-N287P-S322T-N388M-i421aG-C430N-L432F-L434W-S436N-A463G-Y469D | ++++ |
| I283F-P284E-N287Q-S322T-N388M-K394R-C430N-L432F-L434W-S436N-dG462 | ++++ |
| P284E-N287Q-S322T-N388M-K394R-C430N-L432F-L434W-S436N-dG462-Y469S | ++++ |
| P284E-N287Q-S322T-N388M-K394R-C430N-L432F-L434W-S436N-dG462-Y469D | ++++ |
| I283L-N285M-N287T-S322T-A337T-N388M-K394R-C430N-L432F-L434W-S436N-dG462 | ++++ |
| N285M-N287T-S322T-N388M-K394R-C430N-L432F-L434W-S436N-N460G-dG462 | ++++ |
| N285M-N287T-S322T-N388M-K394R-C430N-L432F-L434W-S436N-dG462-A463M | ++++ |
| N285M-N287T-S322T-N388M-K394R-C430N-L432F-L434W-S436N-dG462-A463F | ++++ |
| N285M-N287T-S322T-N388M-K394R-C430N-L432F-L434W-S436N-dG462-A463V | ++++ |
| N285M-N287T-S322T-N388M-K394R-C430N-L432F-L434W-S436N-dG462-Y469S | ++++ |
| N285M-N287T-S322T-N388M-K394R-C430N-L432F-L434W-S436N-dG462-Y469T | ++++ |
| N285M-N287T-S322T-N388M-K394R-C430N-L432F-L434W-S436N-dG462-Y469N | ++++ |
| N285M-N287T-S322T-N388M-K394R-C430N-L432F-L434W-S436N-dG462-A470S | ++++ |
| N285M-N287T-S322T-N388M-K394R-C430N-L432F-L434W-S436N-dA463-A470S | ++++ |
| N285M-N287T-S322T-N388M-K394R-C430N-L432F-L434W-S436N-dA463-S465I | ++++ |
| N285M-N287T-S322T-N388M-K394R-C430N-L432F-L434W-S436N-dA463-S465V | ++++ |
| I283Q-P284E-N287Q-S322T-N388M-K394R-C430N-L432F-L434W-S436N-dA463 | ++++ |
| I283C-P284E-N287Q-S322T-N388M-K394R-C430N-L432F-L434W-S436N-dA463 | ++++ |
| I283L-P284E-N287Q-S322T-N388M-K394R-C430N-L432F-L434W-S436N-dA463 | ++++ |
| P284D-N287Q-S322T-N388M-K394R-C430N-L432F-L434W-S436N-dA463 | ++++ |
| P284E-N287Q-L289H-S322T-N388M-K394R-C430N-L432F-L434W-S436N-dA463 | ++++ |
| P284E-N287Q-L289R-S322T-N388M-K394R-C430N-L432F-L434W-S436N-dA463 | ++++ |
| P284E-N287Q-S322T-N388M-K394R-R422H-C430N-L432F-L434W-S436N-dA463 | ++++ |
| P284E-N287Q-S322T-N388M-K394R-C430N-L432F-L434W-S436N-dA463-A470T | ++++ |
| P284E-N287Q-S322T-N388M-K394R-C430N-L432F-L434W-S436N-G462R-dA463 | ++++ |
| P284E-N287Q-S322T-N388M-K394R-C430N-L432F-L434W-S436N-dA463-S465A | ++++ |
| P284E-N287Q-S322T-N388M-K394R-C430N-L432F-L434W-S436N-dA463-Y469T | ++++ |
| P284E-N287Q-S322T-N388M-K394R-C430N-L432F-L434W-S436N-dA463-Y469E | ++++ |
| P284E-N287Q-S322T-N388M-K394R-C430N-L432F-L434W-S436N-dA463-A470S | ++++ |
| I283T-N285P-E286D-N287T-Q290E-S322T-N388M-K394R-C430A-L432F-L434W-S436N-G462P-A463P | ++++ |
| I283T-N285P-N287T-L289Y-Q290E-S322T-N388M-K394R-C430A-L432F-L434W-S436N-G462P-A463P | ++++ |
| I283T-N285P-N287T-L289A-Q290E-S322T-N388M-K394R-C430A-L432F-L434W-S436N-G462P-A463P | ++++ |
| I283T-N285P-N287T-Q290E-S322T-Q365M-N388M-K394R-C430A-L432F-L434W-S436N-G462P-A463P | ++++ |
| I283T-N285P-N287T-Q290E-S322T-Q365S-N388M-K394R-C430A-L432F-L434W-S436N-G462P-A463P | ++++ |
| I283L-N285P-N287T-Q290E-S322T-N388M-K394R-C430A-L432F-L434W-S436N | ++++ |
| I283H-P284D-N285M-N287T-S322T-N388M-K394R-C430A-L432F-L434W-S436N-G462P-A463P | ++++ |
| I283Q-P284D-N285M-N287T-S322T-N388M-K394R-C430A-L432F-L434W-S436N-G462P-A463P | ++++ |
| I283T-P284D-N285H-N287T-S322T-N388M-K394R-C430A-L432F-L434W-S436N-G462P-A463P | ++++ |
| I283T-P284D-N285V-N287T-S322T-N388M-K394R-C430A-L432F-L434W-S436N-G462P-A463P | ++++ |
| I283T-P284D-N285L-N287T-S322T-N388M-K394R-C430A-L432F-L434W-S436N-G462P-A463P | ++++ |
| I283T-P284D-N285T-N287T-S322T-N388M-K394R-C430A-L432F-L434W-S436N-G462P-A463P | ++++ |
| I283T-P284D-N285S-N287T-S322T-N388M-K394R-C430A-L432F-L434W-S436N-G462P-A463P | ++++ |
| I283T-P284D-N285M-E286N-N287T-S322T-N388M-K394R-C430A-L432F-L434W-S436N-G462P-A463P | ++++ |
| I283T-P284D-N285M-E286A-N287T-S322T-N388M-K394R-C430A-L432F-L434W-S436N-G462P-A463P | ++++ |
| I283T-P284D-N285M-E286G-N287T-S322T-N388M-K394R-C430A-L432F-L434W-S436N-G462P-A463P | ++++ |
| I283T-P284D-N285M-E286H-N287T-S322T-N388M-K394R-C430A-L432F-L434W-S436N-G462P-A463P | ++++ |
| I283T-P284D-N285M-E286D-N287T-S322T-N388M-K394R-C430A-L432F-L434W-S436N-G462P-A463P | ++++ |
| I283T-P284D-N285M-N287T-S288T-S322T-N388M-K394R-C430A-L432F-L434W-S436N-G462P-A463P | ++++ |
| I283T-P284D-N285M-N287T-L289Q-S322T-N388M-K394R-C430A-L432F-L434W-S436N-G462P-A463P | ++++ |
| I283T-P284D-N285M-N287T-L289R-S322T-N388M-K394R-C430A-L432F-L434W-S436N-G462P-A463P | ++++ |
| I283T-P284D-N285M-N287T-L289M-S322T-N388M-K394R-C430A-L432F-L434W-S436N-G462P-A463P | ++++ |
| I283T-P284D-N285M-N287T-S322T-N388M-K394R-C430T-L432F-L434W-S436N-G462P-A463P | ++++ |
| I283T-P284D-N287P-S322T-H384A-N388M-C430N-L432F-L434W-S436N-dG462 | ++++ |
| P25L-I283T-P284D-N287P-S322T-N388M-C430N-L432F-L434W-S436N-dG462 | ++++ |
| I283T-P284D-N287P-S322T-N388M-R422G-C430N-L432F-L434W-S436N-dG462 | ++++ |
| I283T-P284D-N287P-S322T-P368S-N388M-E421V-C430N-L432F-L434W-S436N-dG462 | ++++ |
| I283T-P284D-N287P-S322T-N388M-R422L-C430N-L432F-L434W-S436N-dG462 | ++++ |
| I283T-P284D-N287P-S322T-N388M-C430N-L432F-L434W-S436N-dG462-A463I | ++++ |
| I283T-P284D-N287P-S322T-N388M-C430N-L432F-L434W-S436N-dG462-A463T | ++++ |
| I283T-P284D-N287P-S322T-N388M-C430N-L432F-L434W-S436N-dG462-A463F | ++++ |
| I283T-P284D-N287P-S322T-N388M-C430N-L432F-L434W-S436N-H461V-dG462 | ++++ |
| I283T-P284D-N287P-S322T-N388M-C430N-L432F-L434W-S436N-dG462-S465V | ++++ |
| I283T-P284D-N287P-S322T-N388M-C430N-L432F-L434W-S436N-H461A-dG462 | ++++ |
| I283T-P284D-N287P-S322T-N388M-C430N-L432F-L434W-S436N-H461T-dG462 | ++++ |
| I283T-P284D-N287P-S322T-N388M-C430N-L432F-L434W-S436N-H461Y-dG462 | ++++ |
| I283T-P284D-N287P-S322T-N388M-C430N-L432F-L434W-S436N-dG462-A463S | ++++ |
| I283T-P284D-N287P-S322T-N388M-C430N-L432F-L434W-S436N-H461A-dG462-A463T | ++++ |
| I283T-P284D-N287P-S322T-N388M-C430N-L432F-L434W-S436N-H461A-dG462-S465V | ++++ |
| I283T-P284D-N287P-S322T-N388M-C430N-L432F-L434W-S436N-H461A-dG462-S465I | ++++ |
| I283T-P284D-N287P-S322T-N388M-C430N-L432F-L434W-S436N-H461Y-dG462-A463T | ++++ |
| I283T-P284D-N287P-S322T-N388M-C430N-L432F-L434W-S436N-H461Y-dG462-S465V | ++++ |
| I283T-P284D-N287P-S322T-N388M-C430N-L432F-L434W-S436N-H461V-dG462-A463T | ++++ |
| I283T-P284D-N287P-S322T-N388M-C430N-L432F-L434W-S436N-H461V-dG462-S465V | ++++ |
| I283T-P284D-N287P-S322T-N388M-C430N-L432F-L434W-S436N-dG462-A463T-S465V | ++++ |
| I283T-P284D-N287P-S322T-N388M-C430N-L432F-L434W-S436N-H461A-dG462-A463T-S465V | ++++ |
| I283T-P284D-N287P-S322T-N388M-C430N-L432F-L434W-S436N-H461Y-dG462-A463T-S465V | ++++ |
| I283T-P284D-N287P-S322T-N388M-C430N-L432F-L434W-S436N-H461V-dG462-A463T-S465V | ++++ |
| I283T-P284D-N287P-S322T-N388M-E421V-R422F-C430N-L432F-L434W-S436N-dG462 | ++++ |
| I283T-P284D-N287P-S322T-N388M-E421V-C430N-L432F-L434W-S436N-H461T-dG462 | ++++ |
| I283T-P284D-N287P-S322T-N388M-E421V-C430N-L432F-L434W-S436N-H461V-dG462 | ++++ |
| I283T-P284D-N287P-S322T-N388M-E421V-C430N-L432F-L434W-S436N-dG462-A463T | ++++ |
| I283T-P284D-N287P-S322T-N388M-R422F-C430N-L432F-L434W-S436N-H461T-dG462 | ++++ |
| I283T-P284D-N287P-S322T-N388M-R422F-C430N-L432F-L434W-S436N-H461V-dG462 | ++++ |
| I283T-P284D-N287P-S322T-N388M-R422F-C430N-L432F-L434W-S436N-dG462-A463T | ++++ |
| I283T-P284D-N287P-S322T-N388M-C430N-L432F-L434W-S436N-H461T-dG462-A463T | ++++ |
| I283T-P284D-N287P-S322T-N388M-E421V-R422F-C430N-L432F-L434W-S436N-H461T-dG462 | ++++ |
| I283T-P284D-N287P-S322T-N388M-E421V-R422F-C430N-L432F-L434W-S436N-H461V-dG462 | ++++ |
| I283T-P284D-N287P-S322T-N388M-E421V-R422F-C430N-L432F-L434W-S436N-dG462-A463T | ++++ |
| I283T-P284D-N287P-S322T-N388M-E421V-C430N-L432F-L434W-S436N-H461T-dG462-A463T | ++++ |
| I283T-P284D-N287P-S322T-N388M-E421V-C430N-L432F-L434W-S436N-H461V-dG462-A463T | ++++ |
| I283T-P284D-N287P-S322T-N388M-R422F-C430N-L432F-L434W-S436N-H461T-dG462-A463T | ++++ |
| I283T-P284D-N287P-S322T-N388M-R422F-C430N-L432F-L434W-S436N-H461V-dG462-A463T | ++++ |
| I283T-P284D-N287P-S322T-N388M-E421V-R422F-C430N-L432F-L434W-S436N-H461T-dG462-A463T | ++++ |
| I283T-P284D-N287P-S322T-N388M-E421V-R422F-C430N-L432F-L434W-S436N-H461V-dG462-A463T | ++++ |
| I283T-P284D-N287P-S322T-N388M-E421V-C430N-L432F-L434W-S436N-dG462 | ++++ |
| I283T-P284D-N287P-S322T-N388M-R422F-C430N-L432F-L434W-S436N-dG462 | ++++ |
| I283T-N285P-N287T-Q290E-S322T-N388M-K394R-C430A-L432F-L434W-S436N-dH461 | ++++ |
| I283T-N285P-N287T-Q290E-S322T-N388M-K394R-C430A-L432F-L434W-S436N-dG462 | ++++ |
| I283T-N285P-N287T-Q290E-S322T-N388M-K394R-C430A-L432F-L434W-S436N-dA463 | ++++ |
| I283T-P284D-i284aP-N287P-S322T-N388M-C430N-L432F-L434W-S436N-dG462 | ++++ |
| I283T-P284D-N285M-N287T-S322T-N388M-K394R-C430A-L432F-L434W-S436N-C437M-G462P-A463P | ++++ |
| I283T-P284D-N285M-N287T-S322T-N388M-K394R-C430A-L432F-L434W-S436N-G462P-A463P-I464P | ++++ |
| I283T-P284D-N285M-N287T-L289H-S322T-N388M-K394R-C430A-L432F-L434W-S436N-G462P-A463P | ++++ |
| I283T-P284D-N285I-N287T-i287aS-S322T-N388M-K394R-C430A-L432F-L434W-S436N-G462P-A463P | ++++ |
| I283T-P284D-N285M-N287T-S322T-N388M-K394R-C430A-L432F-L434W-S436N-G462T-A463P | ++++ |
| I283T-N285P-N287T-Q290E-S322T-N388M-K394R-dR422-C430A-L432F-L434W-S436N-dG462 | ++++ |
| L223Y-I283T-N285P-N287T-Q290E-S322T-N388M-K394R-K423G-C430A-L432F-L434W-S436N-dG462 | ++++ |
| I283T-P284D-i284aP-N287P-S322T-N388M-E421V-C430N-L432F-L434W-S436N-dG462-A463T | ++++ |
| I283T-P284D-i284aP-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-H461V-dG462-A463T | ++++ |
| I283T-P284D-i284aP-N287P-S322T-N388M-E421V-R422F-C430N-L432F-L434W-S436N-dG462-A463T | ++++ |
| I283T-P284D-i284aP-N287P-S322T-N388M-E421V-R422F-C430N-L432F-L434W-S436N-H461T-dG462-A463T | ++++ |
| I283T-P284D-i284aP-N287P-S322T-N388M-E421V-R422F-C430N-L432F-L434W-S436N-H461T-dG462 | ++++ |
| I283T-P284D-i284aP-N287P-S322T-N388M-C430N-L432F-L434W-S436N-H461A-dG462-A463T | ++++ |
| I283T-P284D-i284aP-N287P-S322T-N388M-C430N-L432F-L434W-S436N-H461A-dG462 | ++++ |
| E126K-H141Q-I283T-N285P-N287T-Q290E-S322T-N388M-K394R-C430A-L432F-L434W-S436N-dG462 | ++++ |
| P184S-I283T-N285P-N287T-Q290E-S322T-N388M-K394R-C430A-L432F-L434W-S436N-dG462 | ++++ |
| L223Y-I283T-N285P-N287T-Q290E-S322T-N388M-K394R-C430A-L432F-L434W-S436N-dG462 | ++++ |
| V227Y-I283T-N285P-N287T-Q290E-S322T-N388M-K394R-C430A-L432F-L434W-S436N-dG462 | ++++ |
| I283L-N285P-N287T-Q290E-S322T-N388M-K394R-C430A-L432F-L434W-S436N-dG462 | ++++ |
| I283T-N285P-N287T-L289W-Q290E-S322T-N388M-K394R-C430A-L432F-L434W-S436N-dG462 | ++++ |
| I283T-N285P-N287T-L289R-Q290E-S322T-N388M-K394R-C430A-L432F-L434W-S436N-dG462 | ++++ |
| I283T-N285P-N287T-L289C-Q290E-S322T-N388M-K394R-C430A-L432F-L434W-S436N-dG462 | ++++ |
| I283T-N285P-N287T-Q290E-S322T-Q365T-N388M-K394R-C430A-L432F-L434W-S436N-dG462 | ++++ |
| I283T-N285P-N287T-Q290E-S322T-N388M-K394R-K423G-C430A-L432F-L434W-S436N-dG462 | ++++ |
| I283T-N285P-N287T-Q290E-S322T-N388M-K394R-C430V-L432F-L434W-S436N-dG462 | ++++ |
| I283T-N285P-N287T-Q290E-S322T-N388M-K394R-C430A-L432F-L434W-S436N-H444T-dG462 | ++++ |
| I283T-N285P-N287T-Q290E-S322T-N388M-K394R-C430A-L432F-L434W-S436N-H444R-dG462 | ++++ |
| I283T-N285P-N287T-Q290E-S322T-N388M-K394R-C430A-L432F-L434W-S436N-H444F-dG462 | ++++ |
| I283T-N285P-N287T-Q290E-S322T-N388M-K394R-C430A-L432F-L434W-S436N-N460G-dG462 | ++++ |
| I283T-N285P-N287T-Q290E-S322T-N388M-K394R-C430A-L432F-L434W-S436N-dG462-A463F | ++++ |
| I283T-N285P-N287T-Q290E-S322T-N388M-K394R-C430A-L432F-L434W-S436N-dG462-A470S | ++++ |
| I283T-N285P-N287T-Q290E-S322T-N388M-K394R-R422D-C430A-L432F-L434W-S436N-dG462 | ++++ |
| I283T-N285P-N287T-Q290E-S322T-N388M-K394R-C430A-L432F-L434W-S436N-dG462-A463V | ++++ |
| I283T-N285P-N287T-Q290E-S322T-N388M-K394R-C430A-L432F-L434W-S436N-dG462-Y471F | ++++ |
| I35V-Q140L-I283T-N285P-N287T-Q290E-S322T-N388M-K394R-C430A-L432F-L434W-S436N-dG462 | ++++ |
| E224P-I283T-N285P-N287T-Q290E-S322T-N388M-K394R-C430A-L432F-L434W-S436N-dG462 | ++++ |
| E224Q-I283T-N285P-N287T-Q290E-S322T-N388M-K394R-C430A-L432F-L434W-S436N-dG462 | ++++ |
| L222V-I283T-N285P-N287T-Q290E-S322T-N388M-K394R-C430A-L432F-L434W-S436N-dG462 | ++++ |
| V227L-I283T-N285P-N287T-Q290E-S322T-N388M-K394R-C430A-L432F-L434W-S436N-dG462 | ++++ |
| I283T-N285P-E286A-N287T-Q290E-S322T-N388M-K394R-C430A-L432F-L434W-S436N-dG462 | ++++ |
| I283T-N285P-N287T-Q290E-S322T-R361K-N388M-K394R-C430A-L432F-L434W-S436N-dG462 | ++++ |
| I283T-N285P-N287T-Q290E-S322T-M355V-N388M-K394R-C430A-L432F-L434W-S436N-dG462 | ++++ |
| I283T-N285P-N287T-Q290E-S322T-M355T-N388M-K394R-C430A-L432F-L434W-S436N-dG462 | ++++ |
| I283T-N285P-N287T-Q290E-S322T-M355C-N388M-K394R-C430A-L432F-L434W-S436N-dG462 | ++++ |
| I283T-N285P-N287T-Q290E-S322T-N388M-K394R-E421W-C430A-L432F-L434W-S436N-dG462 | ++++ |
| I283T-N285P-N287T-Q290E-S322T-N388S-K394R-C430A-L432F-L434W-S436N-dG462 | ++++ |
| I283T-N285P-N287T-Q290E-S322T-N388M-K394H-C430A-L432F-L434W-S436N-dG462 | ++++ |
| I283T-N285P-N287T-Q290E-S322T-N388M-K394R-H419P-C430A-L432F-L434W-S436N-dG462 | ++++ |
| I283T-N285P-N287T-Q290E-S322T-N388M-K394R-H419A-C430A-L432F-L434W-S436N-dG462 | ++++ |
| I283T-N285P-N287T-Q290E-S322T-N388M-K394R-E421L-C430A-L432F-L434W-S436N-dG462 | ++++ |
| I283T-N285P-N287T-Q290E-S322T-N388M-K394R-S429V-C430A-L432F-L434W-S436N-dG462 | ++++ |
| I283T-N285P-N287T-Q290E-S322T-N388M-K394R-C430A-I431K-L432F-L434W-S436N-dG462 | ++++ |
| I283T-N285P-N287T-Q290E-S322T-N388M-K394R-C430A-I431N-L432F-L434W-S436N-dG462 | ++++ |
| I283T-N285P-N287T-Q290E-S322T-N388M-K394R-C430A-I431P-L432F-L434W-S436N-dG462 | ++++ |
| I283T-N285P-N287T-Q290E-S322T-N388M-K394R-C430A-I431C-L432F-L434W-S436N-dG462 | ++++ |
| I283T-N285P-N287T-Q290E-S322T-N388M-K394R-C430A-I431T-L432F-L434W-S436N-dG462 | ++++ |
| I283T-N285P-N287T-Q290E-S322T-N388M-K394R-C430A-I431A-L432F-L434W-S436N-dG462 | ++++ |
| I283T-N285P-N287T-Q290E-S322T-N388M-K394R-C430S-L432F-L434W-S436N-dG462 | ++++ |
| I283T-N285P-N287T-Q290E-S322T-N388M-K394R-C430A-L432F-L434W-S436N-C437L-dG462 | ++++ |
| I283T-N285P-N287T-Q290E-S322T-N388M-K394R-C430A-L432F-L434W-S436N-Y438W-dG462 | ++++ |
| I283T-N285P-N287T-Q290E-S322T-N388M-K394R-C430A-L432F-L434W-S436N-C437T-dG462 | ++++ |
| I283T-N285P-N287T-Q290E-S322T-N388M-K394R-C430A-L432F-L434W-S436N-C437M-dG462 | ++++ |
| I283T-N285P-N287T-Q290E-S322T-N388M-K394R-C430A-L432F-L434W-S436N-C437I-dG462 | ++++ |
| I283T-N285P-N287T-Q290E-S322T-N388M-K394R-C430A-L432F-L434W-S436N-Y438M-dG462 | ++++ |
| I283T-N285P-N287T-Q290E-S322T-N388M-K394R-C430A-L432F-L434W-S436N-S439P-dG462 | ++++ |
| I283T-N285P-N287T-Q290E-S322T-N388M-K394R-C430A-L432F-L434W-S436N-K447T-dG462 | ++++ |
| D27N-I283T-N285P-N287T-Q290E-S322T-N388M-K394R-C430A-L432F-L434W-S436N-G448T-dG462 | ++++ |
| I283T-N285P-N287T-Q290E-S322T-N388M-K394R-C430A-L432F-L434W-S436N-G448C-dG462 | ++++ |
| I283T-N285P-N287T-Q290E-S322T-N388M-K394R-E421F-C430A-L432F-L434W-S436N-dG462 | ++++ |
| I283T-N285P-N287T-Q290E-S322T-N388M-K394R-i421aD-C430A-L432F-L434W-S436N-dG462 | ++++ |
| I283T-N285P-N287T-Q290E-S322T-N388M-K394R-i421aT-C430A-L432F-L434W-S436N-dG462 | ++++ |
| I283T-N285P-N287T-Q290E-S322T-N388M-K394R-i421aS-C430A-L432F-L434W-S436N-dG462 | ++++ |
| I283T-N285P-N287T-Q290E-S322T-N388M-K394R-i421aI-C430A-L432F-L434W-S436N-dG462 | ++++ |
| I283T-N285P-N287T-Q290E-S322T-N388M-K394R-i421aE-C430A-L432F-L434W-S436N-dG462 | ++++ |
| I283T-N285P-N287T-Q290E-S322T-N388M-K394R-i421aR-C430A-L432F-L434W-S436N-dG462 | ++++ |
| I283T-N285P-N287T-Q290E-S322T-N388M-K394R-i421aP-C430A-L432F-L434W-S436N-dG462 | ++++ |
| I283T-N285P-N287T-Q290E-S322T-N388M-K394R-i424aA-C430A-L432F-L434W-S436N-dG462 | ++++ |
| I283T-N285P-N287T-Q290E-S322T-N388M-K394R-i424aW-C430A-L432F-L434W-S436N-dG462 | ++++ |
| I283T-N285P-N287T-Q290E-S322T-N388M-K394R-i424aL-C430A-L432F-L434W-S436N-dG462 | ++++ |
| I283T-N285P-N287T-Q290E-S322T-N388M-K394R-i424aS-C430A-L432F-L434W-S436N-dG462 | ++++ |
| I283T-N285P-N287T-Q290E-S322T-N388M-K394R-i424aD-C430A-L432F-L434W-S436N-dG462 | ++++ |
| I283T-N285P-N287T-Q290E-S322T-N388M-K394R-i426aL-C430A-L432F-L434W-S436N-dG462 | ++++ |
| I283T-N285P-N287T-Q290E-S322T-N388M-K394R-i429aN-C430A-L432F-L434W-S436N-dG462 | ++++ |
| I283T-N285P-N287T-Q290E-S322T-N388M-K394R-C430A-i430aC-L432F-L434W-S436N-dG462 | ++++ |
| I283T-N285P-N287T-Q290E-S322T-N388M-K394R-C430A-i430aG-L432F-L434W-S436N-dG462 | ++++ |
| I283T-N285P-N287T-Q290E-S322T-N388M-K394R-C430A-i430aN-L432F-L434W-S436N-dG462 | ++++ |
| I283T-N285P-N287T-Q290E-S322T-N388M-K394R-C430A-i430aS-L432F-L434W-S436N-dG462 | ++++ |
| I283T-N285P-N287T-Q290E-S322T-N388M-K394R-C430A-i430aR-L432F-L434W-S436N-dG462 | ++++ |
| I283T-N285P-N287T-Q290E-S322T-N388M-K394R-C430A-i430aL-L432F-L434W-S436N-dG462 | ++++ |
| I283T-N285P-N287T-Q290E-S322T-N388M-K394R-C430A-i430aI-L432F-L434W-S436N-dG462 | ++++ |
| I283T-N285P-N287T-Q290E-S322T-N388M-K394R-C430A-i430aQ-L432F-L434W-S436N-dG462 | ++++ |
| I283T-N285P-N287T-Q290E-S322T-N388M-K394R-C430A-i430aH-L432F-L434W-S436N-dG462 | ++++ |
| I283T-N285P-N287T-Q290E-S322T-N388M-K394R-C430A-L432F-L434W-S436N-i438aD-dG462 | ++++ |
| I283T-N285P-N287T-Q290E-S322T-N388M-K394R-C430A-L432F-L434W-S436N-i443aR-dG462 | ++++ |
| I283T-N285P-N287T-Q290E-S322T-N388M-K394R-C430A-L432F-L434W-S436N-i443aV-dG462 | ++++ |
| I283T-N285P-N287T-Q290E-S322T-N388M-K394R-C430A-L432F-L434W-S436N-i443aL-dG462 | ++++ |
| I283T-N285P-N287T-Q290E-S322T-N388M-K394R-C430A-L432F-L434W-S436N-i443aT-dG462 | ++++ |
| I283T-N285P-N287T-Q290E-S322T-N388M-K394R-C430A-L432F-L434W-S436N-i444aH-dG462 | ++++ |
| I283T-N285P-N287T-Q290E-S322T-N388M-K394R-C430A-L432F-L434W-S436N-i444aP-dG462 | ++++ |
| I283T-N285P-N287T-Q290E-S322T-N388M-K394R-C430A-L432F-L434W-S436N-i444aV-dG462 | ++++ |
| I283T-N285P-N287T-Q290E-S322T-N388M-K394R-C430A-L432F-L434W-S436N-i444aR-dG462 | ++++ |
| I283T-N285P-N287T-Q290E-S322T-N388M-K394R-C430A-L432F-L434W-S436N-i444aN-dG462 | ++++ |
| I283T-N285P-N287T-Q290E-S322T-N388M-K394R-C430A-L432F-L434W-S436N-i444aG-dG462 | ++++ |
| I283T-N285P-N287T-Q290E-S322T-N388M-K394R-C430A-L432F-L434W-S436N-i444aT-dG462 | ++++ |
| I283T-N285P-N287T-Q290E-S322T-N388M-K394R-C430A-L432F-L434W-S436N-i444aL-dG462 | ++++ |
| I283T-N285P-N287T-Q290E-S322T-N388M-K394R-C430A-L432F-L434W-S436N-i444aE-dG462 | ++++ |
| I283T-N285P-N287T-Q290E-S322T-N388M-K394R-C430A-L432F-L434W-S436N-i444aK-dG462 | ++++ |
| I283T-N285P-N287T-Q290E-S322T-N388M-K394R-C430A-L432F-L434W-S436N-i444aS-dG462 | ++++ |
| I283T-N285P-N287T-Q290E-S322T-N388M-K394R-C430A-L432F-L434W-S436N-i445aQ-dG462 | ++++ |
| I283T-N285P-N287T-Q290E-S322T-N388M-K394R-C430A-L432F-L434W-S436N-i447aY-dG462 | ++++ |
| I283T-N285P-N287T-Q290E-S322T-N388M-K394R-C430A-L432F-L434W-S436N-i447aL-dG462 | ++++ |
| I283T-N285P-N287T-Q290E-S322T-N388M-K394R-C430A-L432F-L434W-S436N-i447aK-dG462 | ++++ |
| I283T-N285P-N287T-Q290E-S322T-N388M-K394R-C430A-L432F-L434W-S436N-i447aP-dG462 | ++++ |
| I283T-N285P-N287T-Q290E-S322T-N388M-K394R-C430A-L432F-L434W-S436N-i448aS-dG462 | ++++ |
| I283T-N285P-N287T-Q290E-S322T-N388M-K394R-C430A-i431aG-L432F-L434W-S436N-dG462 | ++++ |
| I283T-N285P-N287T-Q290E-S322T-N388M-K394R-C430A-i431aL-L432F-L434W-S436N-dG462 | ++++ |
| I283T-N285P-N287T-Q290E-S322T-N388M-K394R-C430A-i431aN-L432F-L434W-S436N-dG462 | ++++ |
| I283T-N285P-N287T-Q290E-S322T-N388M-K394R-C430A-L432F-L434W-S436N-i449aS-dG462 | ++++ |
| L223Y-I283T-N285P-N287T-Q290E-S322T-N388M-K394R-E421F-C430A-L432F-L434W-S436N-dG462 | ++++ |
| I283T-N285P-N287T-Q290E-S322T-N388M-K394R-E421F-C430A-i430aG-L432F-L434W-S436N-dG462 | ++++ |
| I283T-N285P-N287T-Q290E-S322T-N388M-K394R-E421F-C430A-i430aL-L432F-L434W-S436N-dG462 | ++++ |
| I283T-N285P-N287T-Q290E-S322T-N388M-K394R-E421F-C430A-i430aN-L432F-L434W-S436N-dG462 | ++++ |
| I283T-N285P-N287T-Q290E-S322T-N388M-K394R-E421F-C430A-L432F-L434W-S436N-i448aS-dG462 | ++++ |
| L223Y-I283T-N285P-N287T-Q290E-S322T-N388M-K394R-E421F-K423G-C430A-L432F-L434W-S436N-dG462 | ++++ |
| L223Y-I283T-N285P-N287T-Q290E-S322T-N388M-K394R-K423G-C430A-i430aG-L432F-L434W-S436N-dG462 | ++++ |
| L223Y-I283T-N285P-N287T-Q290E-S322T-N388M-K394R-K423G-C430A-i430aL-L432F-L434W-S436N-dG462 | ++++ |
| L223Y-I283T-N285P-N287T-Q290E-S322T-N388M-K394R-K423G-C430A-i430aN-L432F-L434W-S436N-dG462 | ++++ |
| L223Y-I283T-N285P-N287T-Q290E-S322T-N388M-K394R-K423G-C430A-L432F-L434W-S436N-i448aS-dG462 | ++++ |
| L223Y-I283T-N285P-N287T-Q290E-S322T-N388M-K394R-i429aN-C430A-L432F-L434W-S436N-dG462 | ++++ |
| L223Y-I283T-N285P-N287T-Q290E-S322T-N388M-K394R-C430A-i430aG-L432F-L434W-S436N-dG462 | ++++ |
| L223Y-I283T-N285P-N287T-Q290E-S322T-N388M-K394R-i429aN-i429bG-C430A-L432F-L434W-S436N-dG462 | ++++ |
| I283T-N285P-N287T-Q290E-S322T-N388M-K394R-C430A-i430aG-L432F-L434W-S436N-C437L-dG462 | ++++ |
| L223Y-I283T-N285P-N287T-Q290E-S322T-N388M-K394R-C430A-i430aG-L432F-L434W-S436N-C437L-dG462 | ++++ |
| L223Y-I283T-N285P-N287T-Q290E-S322T-N388M-K394R-C430A-i430aN-L432F-L434W-S436N-dG462 | ++++ |
| L223Y-I283T-N285P-N287T-Q290E-S322T-N388M-K394R-C430A-L432F-L434W-S436N-C437L-dG462 | ++++ |
| L223Y-I283T-N285P-N287T-Q290E-S322T-N388M-K394R-C430A-i430aL-L432F-L434W-S436N-dG462 | ++++ |
| I283T-N285P-N287T-Q290E-S322T-N388M-K394R-C430A-i430aG-L432F-L434W-S436N-V451S-dG462 | ++++ |
| I283T-N285P-N287T-Q290E-S322T-N388M-K394R-i425aW-C430A-i430aG-L432F-L434W-S436N-dG462 | ++++ |
| H141C-I283T-N285P-N287T-Q290E-S322T-N388M-K394R-C430A-i430aG-L432F-L434W-S436N-dG462 | ++++ |
| L222V-I283T-N285P-N287T-Q290E-S322T-N388M-K394R-C430A-i430aG-L432F-L434W-S436N-dG462 | ++++ |
| I283T-N285P-N287T-Q290E-S322T-I330V-N388M-K394R-C430A-i430aG-L432F-L434W-S436N-dG462 | ++++ |
| I283T-N285P-N287T-Q290E-S322T-N388M-S389H-K394R-C430A-i430aG-L432F-L434W-S436N-dG462 | ++++ |
| H141C-I283T-N285P-N287T-Q290E-S322T-N388M-S389H-K394R-C430A-i430aG-L432F-L434W-S436N-dG462 | ++++ |
| I283T-N285P-N287T-Q290E-S322T-I330V-N388M-S389H-K394R-C430A-i430aG-L432F-L434W-S436N-dG462 | ++++ |
| H141C-I283T-N285P-N287T-Q290E-S322T-I330V-N388M-S389H-K394R-C430A-i430aG-L432F-L434W-S436N-dG462 | ++++ |
| I283T-N285P-N287T-Q290E-S322T-I330V-N388M-S389H-K394R-C430A-i430aG-L432F-L434W-S436N-V451S-dG462 | ++++ |
| E224P-I283T-N285P-N287T-Q290E-S322T-N388M-K394R-C430A-i430aG-L432F-L434W-S436N-dG462 | ++++ |
| L222V-E224P-I283T-N285P-N287T-Q290E-S322T-N388M-K394R-C430A-i430aG-L432F-L434W-S436N-dG462 | ++++ |
| I283T-N285P-N287T-Q290E-S322T-N388M-S389H-K394R-C430A-i430aG-L432F-L434W-S436N-I453L-dG462 | ++++ |
| E224A-I283T-N285P-N287T-Q290E-S322T-N388M-S389H-K394R-C430A-i430aG-L432F-L434W-S436N-dG462 | ++++ |
| E224P-I283T-N285P-N287T-Q290E-S322T-N388M-S389H-K394R-C430A-i430aG-L432F-L434W-S436N-dG462 | ++++ |
| I283T-P284D-i284aP-N287P-S322T-N388M-E421V-i421aR-C430N-L432F-L434W-S436N-H461T-dG462-A463T | ++++ |
| I283T-P284D-i284aP-N287P-S322T-N388M-E421V-i422aK-C430N-L432F-L434W-S436N-H461T-dG462-A463T | ++++ |
| I283T-P284D-i284aP-N287P-S322T-N388M-E421V-C430N-I431V-L432F-L434W-S436N-H461T-dG462-A463T | ++++ |
| I283T-P284D-i284aP-N287P-S322T-Q365I-N388M-E421V-C430N-L432F-L434W-S436N-H461T-dG462-A463T | ++++ |
| I283T-P284D-i284aP-N287P-S322T-Q385G-N388M-E421V-C430N-L432F-L434W-S436N-H461T-dG462-A463T | ++++ |
| I283T-P284D-i284aP-N287P-S322T-Q385L-N388M-E421V-C430N-L432F-L434W-S436N-H461T-dG462-A463T | ++++ |
| I283T-P284D-i284aP-N287P-S322T-Q385S-N388M-E421V-C430N-L432F-L434W-S436N-H461T-dG462-A463T | ++++ |
| I283T-P284D-i284aP-N287P-S322T-Q385T-N388M-E421V-C430N-L432F-L434W-S436N-H461T-dG462-A463T | ++++ |
| I283T-P284D-i284aP-N287P-S322T-H384G-N388M-E421V-C430N-L432F-L434W-S436N-H461T-dG462-A463T | ++++ |
| I283T-P284D-i284aP-N287P-S322T-H384L-N388M-E421V-C430N-L432F-L434W-S436N-H461T-dG462-A463T | ++++ |
| I283T-P284D-i284aP-N287P-L289R-S322T-N388M-E421V-C430N-L432F-L434W-S436N-H461T-dG462-A463T | ++++ |
| I283T-P284D-i284aP-E286A-N287P-S322T-N388M-E421V-C430N-L432F-L434W-S436N-H461T-dG462-A463T | ++++ |
| I283T-P284D-i284aP-E286S-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-H461T-dG462-A463T-D468L | ++++ |
| I283T-P284D-i284aP-N287P-S322T-N388M-E421V-C430N-L432F-L434W-S436N-G448C-H461T-dG462-A463T | ++++ |
| I283T-P284D-i284aP-N287P-S322T-N388M-E421V-C430N-L432F-L434W-S436N-G448E-H461T-dG462-A463T | ++++ |
| I283T-P284D-i284aP-N287P-S322T-N388M-E421V-C430N-L432F-L434W-S436N-G448V-H461T-dG462-A463T | ++++ |
| I283T-P284D-i284aP-N287P-S322T-N388M-E421V-C430N-L432F-L434W-S436N-H461T-dG462-A463T-Y469A | ++++ |
| I283T-P284D-i284aP-N287P-S322T-N388M-E421V-C430N-L432F-L434W-S436N-H461T-dG462-A463T-Y469H | ++++ |
| I283T-P284D-i284aP-N287P-S322T-N388M-E421V-C430N-L432F-L434W-S436N-C437F-H461T-dG462-A463T | ++++ |
| I283T-P284D-i284aP-N287P-S322T-N388M-E421V-C430N-L432F-L434W-S436N-C437G-H461T-dG462-A463T | ++++ |
| I283T-P284D-i284aP-N287P-S322T-N388M-E421V-C430N-i430aR-L432F-L434W-S436N-H461T-dG462-A463T | ++++ |
| I283T-P284D-i284aP-N287P-S322T-N388M-E421V-C430N-I431M-L432F-L434W-S436N-H461T-dG462-A463T | ++++ |
| P45A-I283T-P284D-i284aP-N287P-S322T-N388M-E421V-C430N-L432F-L434W-S436N-i443aG-i443bA-H461T-dG462-A463T | ++++ |
| I283T-P284D-i284aP-N287P-S322T-N388M-E421V-C430N-L432F-L434W-S436N-H461T-G462R-i462aA-A463T | ++++ |
| R15L-I283T-P284D-i284aP-N287P-S322T-K378M-N388M-E421V-C430N-L432F-L434W-S436N-H461T-dG462-A463T | ++++ |
| V272L-I283T-P284D-i284aP-N287P-S322T-N388M-E421V-C430N-L432F-L434W-S436N-H461T-dG462-A463T | ++++ |
| V272R-I283T-P284D-i284aP-N287P-S322T-N388M-E421V-C430N-L432F-L434W-S436N-H461T-dG462-A463T | ++++ |
| V272I-I283T-P284D-i284aP-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-C437F-G448E-H461T-dG462-A463T | ++++ |
| I283T-P284D-i284aP-N287P-S322T-N388M-E421V-C430N-L432F-L434W-S436N-C437F-G448E-H461T-G462R-i462aA-A463T | ++++ |
| I283T-P284D-i284aP-N287P-S322T-N388M-E421V-C430N-L432F-L434W-S436N-C437F-H461T-G462R-i462aA-A463T | ++++ |
| I283T-P284D-i284aP-N287P-S322T-N388M-E421V-C430N-L432F-L434W-S436N-G448E-H461T-G462R-i462aA-A463T | ++++ |
| V272R-I283T-P284D-i284aP-N287P-S322T-N388M-E421V-C430N-L432F-L434W-S436N-C437F-H461T-dG462-A463T | ++++ |
| V272R-I283T-P284D-i284aP-N287P-S322T-N388M-E421V-C430N-L432F-L434W-S436N-C437F-G448E-H461T-dG462-A463T | ++++ |
| V272R-I283T-P284D-i284aP-N287P-S322T-N388M-E421V-C430N-L432F-L434W-S436N-C437F-G448E-H461T-G462R-i462aA-A463T | ++++ |
| V272R-I283T-P284D-i284aP-N287P-S322T-N388M-E421V-C430N-L432F-L434W-S436N-C437F-H461T-G462R-i462aA-A463T | ++++ |
| V272R-I283T-P284D-i284aP-N287P-S322T-N388M-E421V-C430N-L432F-L434W-S436N-G448E-H461T-dG462-A463T | ++++ |
| V272R-I283T-P284D-i284aP-N287P-S322T-N388M-E421V-C430N-L432F-L434W-S436N-G448E-H461T-G462R-i462aA-A463T | ++++ |
| V272R-I283T-P284D-i284aP-N287P-S322T-N388M-E421V-C430N-L432F-L434W-S436N-H461T-G462R-i462aA-A463T | ++++ |
| V272I-I283T-P284D-i284aP-N287P-S322T-N388M-E421V-C430N-L432F-L434W-S436N-C437F-H461T-G462R-i462aA-A463T | ++++ |
| V272K-I283T-P284D-i284aP-N287P-S322T-N388M-E421V-C430N-L432F-L434W-S436N-C437F-H461T-G462R-i462aA-A463T | ++++ |
| A33P-I283T-P284D-i284aP-N287P-S322T-N388M-E421V-C430N-L432F-L434W-S436N-C437F-H461T-G462R-i462aA-A463T | ++++ |
| I283T-P284D-i284aP-N287P-S322T-H384S-N388M-E421V-C430N-L432F-L434W-S436N-C437F-H461T-G462R-i462aA-A463T | ++++ |
| I283T-P284D-i284aP-N287P-S322T-N388M-I418V-E421V-C430N-L432F-L434W-S436N-C437F-H461T-G462R-i462aA-A463T | ++++ |
| K43E-I283T-P284D-i284aP-N287P-S322T-N388M-E421V-C430P-L432F-L434W-S436N-C437F-H461T-G462R-i462aA-A463T | ++++ |
| I283T-P284D-i284aP-N287P-S322T-N388M-E421V-C430R-L432F-L434W-S436N-C437F-H461T-G462R-i462aA-A463T | ++++ |
| I283T-P284D-i284aP-N287P-S322T-N388M-E421V-C430N-L432F-L434W-S436N-C437F-Y438L-H461T-G462R-i462aA-A463T | ++++ |
| I283T-P284D-i284aP-N287P-S322T-N388M-E421V-C430N-L432F-L434W-S436Q-C437F-H461T-G462R-i462aA-A463T | ++++ |
| I283T-P284D-i284aP-N287P-S322T-N388M-E421V-C430N-L432F-L434W-S436N-C437F-E441V-H461T-G462R-i462aA-A463T | ++++ |
| I283T-P284D-i284aP-N287P-S322T-N388M-E421V-C430N-L432F-L434W-S436N-C437F-i444aT-H461T-G462R-i462aA-A463T | ++++ |
| I283T-P284D-i284aP-N287P-S322T-N388M-E421V-C430R-L432F-L434W-S436N-C437F-Y438L-H461T-G462R-i462aA-A463T | ++++ |
| V272I-I283T-P284D-i284aP-N287P-S322T-N388M-E421V-C430R-L432F-L434W-S436N-C437F-H461T-G462R-i462aA-A463T | ++++ |
| V272I-I283T-P284D-i284aP-N287P-S322T-N388M-E421V-C430R-L432F-L434W-S436N-C437F-Y438L-H461T-G462R-i462aA-A463T | ++++ |
| V272I-I283T-P284D-i284aP-N287P-S322T-N388M-E421V-C430N-L432F-L434W-S436N-C437F-Y438L-H461T-G462R-i462aA-A463T | ++++ |
| V272R-I283T-P284D-i284aP-N287P-S322T-N388M-I418V-E421V-C430N-L432F-L434W-S436N-G448E-H461T-G462R-i462aA-A463T | ++++ |
| G17S-V272R-I283T-P284D-i284aP-N287P-S322T-N388M-E421K-C430N-L432F-L434W-S436N-G448E-H461T-G462R-i462aA-A463T | ++++ |
| V272R-I283T-P284D-i284aP-N287P-S322T-N388M-E421V-E428T-C430N-L432F-L434W-S436N-G448E-H461T-G462R-i462aA-A463T | ++++ |
| V272R-I283T-P284D-i284aP-N287P-S322T-N388M-E421V-C430N-L432F-L434W-S436N-E441H-G448E-H461T-G462R-i462aA-A463T | ++++ |
| V46A-V272R-I283T-P284D-i284aP-N287P-S322T-N388M-E421V-C430N-L432F-L434W-S436N-G448E-i458aG-H461T-G462R-i462aA- | ++++ |
| A463T | |
| S39G-V272R-I283T-P284D-i284aP-N287P-S322T-N388M-E421V-F426L-E428T-C430N-L432F-L434W-S436N-G448E-H461T-G462R- | ++++ |
| i462aA-A463T | |
| V272R-I283T-P284D-i284aP-N287P-S322T-N388M-E421V-E428T-C430N-L432F-L434W-S436N-K447I-G448E-H461T-G462R-i462aA- | |
| A463T | ++++ |
| E224P-I283T-N285P-N287T-Q290E-S322T-N388M-S389H-K394R-C430A-i430aG-L432F-L434W-S436N-I453L-dG462 | ++++ |

Preferably, any of the above described variants having multiple mutations further has one or more substitutions, deletions and/or insertions in comparison to the corresponding sequence from which it is derived and wherein these substitutions, deletions and/or insertions occur at one or more of the positions corresponding to positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470, 471, 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463 and 469 in the amino acid sequence shown in SEQ ID NO:1.

The present invention also relates to a method for providing a variant of an MDC wherein said variant shows an improved activity of converting 3-methylcrotonic acid into isobutene, said method comprising the step of effecting one or more changes in the sequence of the MDC wherein said change(s) is/are effected at one or more amino acid positions selected from the group consisting of the amino acid positions corresponding to positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470 and 471 in the amino acid sequence shown in SEQ ID NO:1. “Corresponding to” means corresponding to any of these positions in a related sequence.

As regards the preferred embodiments of an MDC to be mutated according to such a method, the same applies as has been set forth herein-above.

In one preferred embodiment the MDC from which the MDC variant is derived is an MDC which shows the amino acid sequence as shown in SEQ ID NO:1 or an amino acid sequence having at least 55%, 60%, 70%, 80%, 81%, 82%, 83%, 84%, 85%, 86%, 87%, 88%, 89% or 90% sequence identity to SEQ ID NO:1 or any of the preferred degrees of sequence identity as specified herein above.

Moreover, as regards preferred embodiments of the degree of improvement in activity and the changes to be effected, the same applies as described herein above.

The change(s) which is/are effected at any of the above position(s) is/are substitution(s), deletion(s) and/or insertion(s) as defined herein above.

An MDC variant of the present invention can be fused to a homologous or heterologous polypeptide or protein, an enzyme, a substrate or a tag to form a fusion protein. Fusion proteins in accordance with the present invention will have the same improved activity as the MDC variant of the present invention. Polypeptides, enzymes, substrates or tags that can be added to another protein are known in the art. They may useful for purifying or detecting the proteins of the invention. For instance, tags that can be used for detection and/or purification are e.g. FLAG-tag, His6-tag or a Strep-tag. Alternatively, the protein of the invention can be fused to an enzyme e.g. luciferase, for the detection or localisation of said protein. Other fusion partners include, but are not limited to, bacterial β-galactosidase, trpE, Protein A, β-lactamase, alpha amylase, alcohol dehydrogenase or yeast alpha mating factor. It is also conceivable that the polypeptide, enzyme, substrate or tag is removed from the protein of the invention after e.g. purification. Fusion proteins can typically be made by either recombinant nucleic acid methods or by synthetic polypeptide methods known in art.

The present invention further relates to a nucleic acid molecule encoding an MDC variant of the present invention and to a vector comprising said nucleic acid molecules. Vectors that can be used in accordance with the present invention are known in the art. The vectors can further comprise expression control sequences operably linked to the nucleic acid molecules of the present invention contained in the vectors. These expression control sequences may be suited to ensure transcription and synthesis of a translatable RNA in bacteria or fungi. Expression control sequences can for instance be promoters. Promoters for use in connection with the nucleic acid molecules of the present invention may be homologous or heterologous with regard to its origin and/or with regard to the gene to be expressed. Suitable promoters are for instance promoters which lend themselves to constitutive expression. However, promoters which are only activated at a point in time determined by external influences can also be used. Artificial and/or chemically inducible promoters may be used in this context.

Preferably, the vector of the present invention is an expression vector. Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-ß-D-thiogalactopyranoside) (deBoer et al., Proc. Natl. Acad. Sci. USA 80 (1983), 21-25). Termination signals for transcription are also described in the literature.

In addition, the present invention relates to a host cell comprising the nucleic acid molecule or the vector of the present invention.

In a preferred embodiment, the host cell according to the presenting invention is a microorganism, in particular a bacterium or a fungus. In a more preferred embodiment, the host cell of the present invention is E. coli, a bacterium of the genus Clostridium or a yeast cell, such as S. cerevisiae. In another preferred embodiment the host cell is a plant cell or a non-human animal cell.

The transformation of the host cell with a vector according to the invention can be carried out by standard methods, as for instance described in Sambrook and Russell (2001), Molecular Cloning: A Laboratory Manual, CSH Press, Cold Spring Harbor, NY, USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc.

As mentioned above, the enzymatic conversion of 3-methylcrotonic acid into isobutene utilizing an MDC is preferably performed in the presence of a Flavin prenyltransferase and relies on a reaction of two consecutive steps catalyzed by the two enzymes, i.e., the MDC (catalyzing the actual decarboxylation of 3-methylcrotonic acid into isobutene) with a Flavin prenyltransferase which provides the modified flavin cofactor. The flavin cofactor may preferably be FMN or FAD. FMN (flavin mononucleotide; also termed riboflavin-5′-phosphate) is a biomolecule produced from riboflavin (vitamin B2) by the enzyme riboflavin kinase and functions as prosthetic group of various reactions. FAD (flavin adenine dinucleotide) is a redox cofactor, more specifically a prosthetic group, involved in several important reactions in metabolism. Thus, in a preferred embodiment, when producing isobutene from 3-methylcrotonic acid comprising the step of incubating an MDC variant of the invention with 3-methylcrotonic acid a Flavin prenyltransferase is present which, in a first step, modifies a flavin cofactor (FMN or FAD) into a (modified) flavin-derived cofactor. Flavin prenyltransferase prenylates the flavin ring of the flavin cofactor (FMN or FAD) into a (modified) prenylated flavin cofactor. This reaction is schematically illustrated in FIG. 1A.

In a second step, the actual conversion of 3-methylcrotonic acid into isobutene is catalyzed by said MDC variant via a 1,3-dipolar cycloaddition based mechanism wherein said FMN-dependent decarboxylase uses the prenylated flavin cofactor (FMN or FAD) provided by the associated Flavin prenyltransferase. This reaction is schematically illustrated in FIG. 1B.

Thus, preferably, the host cell of the present invention is a cell which expresses an Flavin prenyltransferase capable of modifying a flavin cofactor (FMN or FAD) into a (modified) flavin-derived cofactor. In a preferred embodiment, the host cell is a cell which naturally (endogenously) expresses a Flavin prenyltransferase. In another preferred embodiment, the host cell is a cell which recombinantly expresses a Flavin prenyltransferase by, e.g., introducing a nucleic acid molecule encoding a Flavin prenyltransferase or a vector comprising such a nucleic acid molecule.

In a preferred embodiment, said Flavin prenyltransferase which modifies the flavin cofactor (FMN or FAD) into a (modified) flavin-derived cofactor is a phenylacrylic acid decarboxylase (PAD)-type protein, or the closely related prokaryotic enzyme UbiX, an enzyme which is involved in ubiquinone biosynthesis in prokaryotes.

In Escherichia coli, the protein UbiX (also termed Flavin prenyltransferase) has been shown to be involved in the third step of ubiquinone biosynthesis:

3-octaprenyl-4-hydroxybenzoate [symbol] 2-octaprenylphenol+CO₂.

Thus, in a preferred embodiment, the modification of a flavin cofactor (FMN or FAD) into the corresponding (modified) flavin-derived cofactor is catalyzed by the FMN-containing protein phenylacrylic acid decarboxylase (PAD). The enzymes involved in the modification of the flavin cofactor (FMN or FAD) into the corresponding modified flavin-derived cofactor were initially annotated as decarboxylases (EC 4.1.1.-). Some phenylacrylic acid decarboxylases (PAD) are now annotated as flavin prenyl transferases as EC 2.5.1.-.

In a more preferred embodiment, the conversion of 3-methylcrotonic acid into isobutene makes use of a phenylacrylic acid decarboxylase (PAD)-type protein as the Flavin prenyltransferase which modifies a flavin cofactor (FMN or FAD) into the corresponding (modified) flavin-derived cofactor wherein said phenylacrylic acid decarboxylase (PAD)-type protein is derived from Candida albicans (Uniprot accession number Q5A8L8), Aspergillus niger (Uniprot accession number A3F715), Saccharomyces cerevisiae (Uniprot accession number P33751) or Cryptococcus gattii (Uniprot accession number E6R9Z0).

In a preferred embodiment, the phenylacrylic acid decarboxylase (PAD)-type protein employed in the method of the present invention is a phenylacrylic acid decarboxylase (PAD)-type protein derived from Candida albicans (Uniprot accession number Q5A8L8; SEQ ID NO:3), Aspergillus niger (Uniprot accession number A3F715; SEQ ID NO: 4), Saccharomyces cerevisiae (Uniprot accession number P33751; SEQ ID NO: 5) or Cryptococcus gattii (Uniprot accession number E6R9Z0; SEQ ID NO:6) having the amino acid sequence as shown in SEQ ID NO:3, SEQ ID NO:4, SEQ ID NO: 5 and SEQ ID NO:6, respectively.

In a preferred embodiment of the present invention the phenylacrylic acid decarboxylase (PAD)-type protein is an enzyme comprising an amino acid sequence selected from the group consisting of SEQ ID NOs: 3 to 6 or a sequence which is at least n % identical to any of SEQ ID NOs: 3 to 6 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of modifying a flavin cofactor (FMN or FAD) into the corresponding (modified) flavin-derived cofactor.

As regards the determination of sequence identity, the same applies as has been set forth above.

In another preferred embodiment, the modification of a flavin cofactor (FMN or FAD) into the corresponding (modified) flavin-derived cofactor is catalyzed by the Flavin prenyltransferase also termed UbiX (initially annotated EC 4.1.1.-). As mentioned above, the enzymes involved in the modification of the flavin cofactor (FMN or FAD) into the corresponding modified flavin-derived cofactor were initially annotated as decarboxylases. Some phenylacrylic acid decarboxylases (PAD) are now annotated as flavin prenyl transferases as EC 2.5.1.-.

In a more preferred embodiment, the conversion of 3-methylcrotonic acid into isobutene makes use of a Flavin prenyltransferase (also termed UbiX) as the Flavin prenyltransferase which modifies the flavin cofactor (FMN or FAD) into the corresponding (modified) flavin-derived cofactor wherein said Flavin prenyltransferase (also termed UbiX) is derived from Escherichia coli (Uniprot accession number P0AG03), Bacillus subtilis (Uniprot accession, number A0A086WXG4), Pseudomonas aeruginosa (Uniprot accession number A0A072ZCW8) or Enterobacter sp. DC4 (Uniprot accession number W7P6B1).

In an even more preferred embodiment, the Flavin prenyltransferase (also termed UbiX) employed in the method of the present invention is a Flavin prenyltransferase (also termed UbiX) derived from Escherichia coli (Uniprot accession number P0AG03; SEQ ID NO:2), Bacillus subtilis (Uniprot accession, number A0A086WXG4; SEQ ID NO: 7), Pseudomonas aeruginosa (Uniprot accession number A0A072ZCW8; SEQ ID NO: 8) or Enterobacter sp. DC4 (Uniprot accession number W7P6B1; SEQ ID NO:9) having the amino acid sequence as shown in SEQ ID NO:2, SEQ ID NO:7, SEQ ID NO: 8 and SEQ ID NO:9, respectively.

In a preferred embodiment of the present invention the Flavin prenyltransferase is an enzyme comprising an amino acid sequence selected from the group consisting of SEQ ID NOs: 2 and 7 to 9 or a sequence which is at least n % identical to any of SEQ ID NOs: 2 and 7 to 9 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of modifying a flavin cofactor (FMN or FAD) into the corresponding (modified) flavin-derived cofactor. As regards the determination of the sequence identity, the same applies as has been set forth above.

The present invention also relates to a method for producing isobutene from 3-methylcrotonic acid comprising the step of incubating an MDC variant of the invention with 3-methylcrotonic acid under conditions allowing said conversion (preferably further in the presence of a Flavin prenyltransferase as described above) or comprising the step of culturing a host cell of the present invention expressing an MDC variant (and preferably further expressing a Flavin prenyltransferase as described above) in a suitable medium and recovering the produced isobutene.

It is also conceivable in this context that in such a method not only one enzyme according to the present invention is employed but a combination of two or more enzymes.

The present invention also relates to the use of an MDC variant or a host cell of the present invention as described above for the conversion of 3-methylcrotonic acid into isobutene, preferably in the presence of a Flavin prenyltransferase or in the presence of a host co-expressing a Flavin prenyltransferase as described herein above. Moreover, in a further embodiment, the present invention relates to a method for producing isobutene from 3-methylcrotonic acid by bringing 3-methylcrotonic acid into contact with the MDC variant of the present invention, preferably in the presence of a Flavin prenyltransferase, or with a host cell comprising a nucleic acid molecule encoding the MDC variant of the present invention, wherein said host cell preferably expresses a Flavin prenyltransferase. Thus, in a preferred embodiment, the present invention relates to a method for converting 3-methylcrotonic acid into isobutene comprising the steps of: (i) culturing the above-described host cell of the invention in a suitable medium; and (ii) achieving the production of isobutene from 3-methylcrotonic acid.

Thus, in a preferred embodiment, the present invention relates to methods and uses utilizing a host cell of the present invention which expresses an MDC variant of the present invention and, preferably, further expressing a Flavin prenyltransferase as described herein above.

In another preferred embodiment, such a host cell is an organism which is capable of producing 3-methylcrotonic acid.

In another preferred embodiment, the method according to the invention is carried out in culture, in the presence of an organism, preferably a microorganism, producing an enzyme variant of the present invention and, preferably, also producing a Flavin prenyltransferase. In such an embodiment of the invention, an organism, preferably a microorganism, that produces an enzyme of the present invention and, preferably, also producing a Flavin prenyltransferase, is used. In a preferred embodiment, the (micro) organism is recombinant in that the enzyme produced by the host is heterologous relative to the production host. The method can thus be carried out directly in the culture medium, without the need to separate or purify the enzymes. In an especially advantageous manner, a (micro) organism is used having the natural or artificial property of endogenously producing 3-methylcrotonic acid so as to produce isobutene directly from the substrate already present in the culture in solution.

In connection with the above described methods and uses, the microorganisms are cultivated under suitable culture conditions allowing the occurrence of the enzymatic reaction of the MDC variants of the present invention (and, preferably, also the Flavin prenyltransferases as described above). The specific culture conditions depend on the specific microorganism employed but are well known to the person skilled in the art. The culture conditions are generally chosen in such a manner that they allow the expression of the genes encoding the MDC variant of the present invention (and, preferably, also a Flavin prenyltransferase as described above). Various methods are known to the person skilled in the art in order to improve and fine-tune the expression of certain genes at certain stages of the culture such as induction of gene expression by chemical inducers or by a temperature shift.

In another embodiment, the above described methods of the invention comprise the step of providing the organism, preferably the microorganism carrying the respective enzyme activity or activities in the form of a (cell) culture, preferably in the form of a liquid cell culture, a subsequent step of cultivating the organism, preferably the microorganism in a fermenter (often also referred to a bioreactor) under suitable conditions allowing the expression of the respective enzyme and further comprising the step of effecting an enzymatic conversion of a method of the invention as described herein above. Suitable fermenter or bioreactor devices and fermentation conditions are known to the person skilled in the art. A bioreactor or a fermenter refers to any manufactured or engineered device or system known in the art that supports a biologically active environment. Thus, a bioreactor or a fermenter may be a vessel in which a chemical/biochemical process like the method of the present invention is carried out which involves organisms, preferably microorganisms and/or biochemically active substances, i.e., the enzyme(s) described above derived from such organisms or organisms harboring the above described enzyme(s). In a bioreactor or a fermenter, this process can either be aerobic or anaerobic. These bioreactors are commonly cylindrical and may range in size from litres to hundreds of cubic meters and are often made of stainless steel. In this respect, without being bound by theory, the fermenter or bioreactor may be designed in a way that it is suitable to cultivate the organisms, preferably microorganisms, in, e.g., a batch-culture, feed-batch-culture, perfusion culture or chemostate-culture, all of which are generally known in the art.

The culture medium can be any culture medium suitable for cultivating the respective organism or microorganism.

In yet a further embodiment, the method according to the invention can be carried out in vitro, e.g. in the presence of isolated enzyme or of cell lysates comprising the enzyme or partially purified enzyme preparations comprising the MDC variant of the present invention (and, preferably, also a Flavin prenyltransferase as described above). In vitro preferably means in a cell-free system.

In one embodiment, the enzyme(s) employed in the method is (are) used in purified form. However, such a method may be costly, since enzyme and substrate production and purification costs are high.

Thus, in another preferred embodiment, the enzymes employed in the method are present in the reaction as a non-purified extract, or else in the form of non-lysed bacteria, so as to economize on protein purification costs. However, the costs associated with such a method may still be quite high due to the costs of producing and purifying the substrates.

In an in vitro reaction the enzymes, native or recombinant, purified or not, are incubated in the presence of the substrate in physicochemical conditions allowing the enzymes to be active, and the incubation is allowed to proceed for a sufficient period of time allowing production of the desired product as described above. At the end of the incubation, one optionally measures the presence of isobutene by using any detection system known to one of skill in the art such as gas chromatography or colorimetric tests for measuring the formation of isobutene.

In a particularly preferred embodiment of the invention the method is carried out in vitro and the enzyme is immobilized. Means and methods for immobilizing enzymes on different supports are well-known to the person skilled in the art.

The method according to the invention furthermore comprises the step of collecting gaseous products, i.e. isobutene, degassing out of the reaction, i.e. recovering the product which degasses, e.g., out of the culture. Thus, in a preferred embodiment, the method is carried out in the presence of a system for collecting isobutene under gaseous form during the reaction.

As a matter of fact, isobutene adopts the gaseous state at room temperature and atmospheric pressure. Moreover, isobutene also adopts the gaseous state under culture conditions at 37° C. The method according to the invention therefore does not require extraction of isobutene from the liquid culture medium, a step which is always very costly when performed at industrial scale. The evacuation and storage of gaseous hydrocarbons, in particular of isobutene, and their possible subsequent physical separation and chemical conversion can be performed according to any method known to one of skill in the art.

Finally, the present invention relates to a composition comprising a variant of an MDC of the present invention, a nucleic acid molecule of the present invention, a vector of the present invention or a host cell of the present invention. As regards the variant of an MDC, the nucleic acid molecule, the vector or the host cell, the same applies as has been set forth above in connection with the methods according to the present invention.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

FIG. 1A: shows a schematic reaction of the enzymatic prenylation of a flavin mononucleotide (FMN) into the corresponding modified (prenylated) flavin cofactor.

FIG. 1B: Schematic reaction of the enzymatic conversion of 3-methylcrotonic acid into isobutene.

The invention will now be described by reference to the following examples which are merely illustrative and are not to be construed as a limitation of the scope of the present invention.

### EXAMPLES

### Example 1: Identification of Variants of a Ferulic Acid Decarboxylase (UbiD-Like) Enzyme from Yersinia frederiksenii with Increased Activity for the Reaction of Conversion of 3-Methylcrotonic Acid (3MC) into Isobutene (IBN)

The Ferulic acid decarboxylase enzyme of Yersinia frederiksenii ((UniProt ID A0A0T9UUQ9, RefSeq WP_050108772.1; SEQ ID NO:1) is capable of catalysing, amongst other reactions, the decarboxylation of 3-methylcrotonic acid (3MC) into isobutene (IBN). A directed evolution approach was used in order to specifically improve the catalytic efficiency of this reaction. This approach consisted in (1) the design of assay systems to test the activity of enzyme variants, (2) the generation of collections of single point or multiple mutants for Yersinia frederiksenii Ferulic acid decarboxylase, and (3) the use of the activity assays to screen the collection of mutants in order to identify variants with improved activity compared to the activity of the wild type Yersinia frederiksenii Ferulic acid decarboxylase.

This approach led to the identification and characterization of a collection of mutants with increased activity compared to the wild type enzyme.

### Materials and Methods

### A) Cloning of Ferulic Acid Decarboxylase Enzymes

The polynucleotide sequences coding for the different mutants identified during the evolution of the Yersinia frederiksenii Ferulic acid decarboxylase enzyme were generated using a range of standard molecular biology techniques. All these techniques used a codon-optimised polynucleotide sequence for expression in Escherichia coli as template.

In particular, all the enzyme encoding polynucleotide sequences were codon-optimized for the expression in Escherichia coli and subsequently chemically synthesized. They were then cloned in a pET25 (Novagen) expression vector or fused with a polynucleotide tag in 5′ or 3′ coding for a 6His purification tag before being cloned in a pET25 expression vector.

### B) Construction of Ferulic Acid Decarboxylase Mutants

The polynucleotide sequences coding for the different mutants identified during the evolution of the selected Ferulic acid decarboxylase enzymes were generated using a range of standard molecular biology techniques.

Different PCR-based techniques known in the art were used for the construction of single-point mutants. For the generation of enzyme variants bearing multiple mutations (at least two mutations), either PCR-based techniques or other methods known in the art were used to introduce these mutations.

Following mutagenesis, the mutated polynucleotide sequence was inserted into a pET25 expression vector with or without fusion to a tag as described above either using standard ligase-based subcloning techniques, whole plasmid extension by PCR or ligase-independent cloning techniques.

C) Selection of the Enzyme Mutants with Increased Activity

### In Vitro Assay in 384-Deepwell Microplates Based on Exogenous 3-Methylcrotonate (3MC) (VITRO384)

This assay is based on the use of a bacterial strain (BL21 (DE3), Novagen) transformed with two expression vectors leading to the production of the last two enzymes involved in the metabolic pathway converting 3MC to isobutene; namely the Flavin prenyltransferase UbiX protein from E. coli cloned in a pRSFDuet™ (Novagen) expression vector and the variant Ferulic acid decarboxylase from Yersinia frederiksenii cloned in one of the above expression vectors. This strain is first plated out onto LB-agar plates supplemented with the appropriate antibiotics. Cells were grown overnight at 32° C. until individual colonies reach the desired size. Single colonies were then picked and individually transferred into 50 μL of liquid LB medium supplemented with the appropriate antibiotic. Cell growth is carried out with shaking for 21 hours at 34° C. The LB cultures were used to inoculate 300 μL in 384 deepwell microplates of auto-induction medium (Studier F W, Prat. Exp. Pur. 41, (2005), 207-234) supplemented with the appropriate antibiotic and grown in a shaking incubator set at 700 rpm and 85% humidity for 24 h at 34° C. in order to produce the two types of recombinant enzymes. The cell pellet containing these two overexpressed recombinant enzymes is then resuspended in 30 μL of lysis mix (pH 7.5, Phosphate 50 mM, NaCl 20 mM, MgCl₂ 2 mM, Lysozyme 1 mg/mL, DNAse 0.03 mg/mL) and incubated for 1 hour in a shaking incubator at 34° C., 700 rpm. The mix is then supplemented with 10 μL of reaction mix (final composition: pH 7.5, Phosphate 50 mM, NaCl 20 mM, MgCl₂ 2 mM, Lysozyme 0.75 mg/mL, DNAse 0.0225 mg/mL, KCl 100 mM) supplemented with 1 to 200 mM (final) 3MC and incubated for a further 1 to 4 hours in a shaking incubator at 34° C., 700 rpm. During this step, the MDC enzyme catalyzes the decarboxylation of 3MC into IBN. After 5 to 10 min inactivation at 80° C. or 90° C., the IBN produced is quantified by gas chromatography as followed. 100 μL of headspace gases from each enzymatic reaction are injected in a Brucker GC-450 system equipped with a Flame Ionization Detector (FID). Compounds present in samples were separated by chromatography using a RTX-1 columns at 100° C. with a 1 mL·min⁻¹ constant flow of nitrogen as carrier gas. Upon injection, peak areas of isobutene were calculated.

d) Identification of Variants of Yersinia frederiksenii Ferulic Acid Decarboxylase with Increased Activity for the Reaction of Conversion of 3-Methylcrotonic Acid into Isobutene

The Ferulic acid decarboxylase gene presenting a capacity to catalyze the reaction of conversion of 3MC into IBN (UniProt ID A0A0T9UUQ9, RefSeq WP_050108772.1 from Yersinia frederiksenii, SEQ ID NO: 1) was subjected to directed mutagenesis in order to create single point mutations, multiple mutations, deletions or insertions variants. Each of these variants were subsequently tested for their increased activity to convert 3MC in IBN, using the previously described assay. 841 variants presented an increased capacity to convert 3MC into IBN.

The list of improved variants is presented in the following Table 2. The increase in activity is described relative to the wild-type enzyme (with “+” representing a low increase in activity and “++++” representing a high increase in activity). The mutations are described as followed: R387A means the wild-type amino-acid arginine (R) at position 387 is replaced by an alanine (A); dG462 means the wild-type amino-acid glycine (G) at position 462 is deleted; i443aR means an arginine (R) has been inserted after the amino-acid at position 443 (if a second amino-acid is inserted, it will be annotated with i443b).

TABLE 1
List of Yersinia frederiksenii as Ferulic acid decarboxylase variants presenting an increase in isobutene production from 3-methylcrotonic acid.

| | Activity |
|---|---|
| | relative |
| Mutants | to WT |
| R387A | + |
| L432A | + |
| L434V | + |
| S436A | + |
| G380V-K391L-L432W-L434M | + |
| L432F-L434W | + |
| L432F-L434F | + |
| L432W-L434W | + |
| K391I-L432W-L434M | + |
| L432A-S436A | + |
| L432F-L434W-S436A | + |
| R387A-L432F-L434W-S436A | + |
| N388M-L432F-L434W | + |
| K394R-L432F-L434W | + |
| C430S-L432F-L434W | + |
| L432F-L434W-S436N | + |
| N388M-L432F-L434W-S436A | + |
| C430P-L432F-L434W-S436A | + |
| C430S-L432F-L434W-S436A | + |
| K394R-L432F-L434W-S436A | + |
| N388M-L432F-L434W-S436N | + |
| C430P-L432F-L434W-S436N | + |
| C430S-L432F-L434W-S436N | + |
| K394R-L432F-L434W-S436N | + |
| N388M-C430P-L432F-L434W-S436A | + |
| N388M-C430S-L432F-L434W-S436A | + |
| N388M-K394R-L432F-L434W-S436A | + |
| K394R-C430P-L432F-L434W-S436A | + |
| K394R-C430S-L432F-L434W-S436A | + |
| N388M-C430P-L432F-L434W-S436N | + |
| N388M-C430S-L432F-L434W-S436N | + |
| N388M-K394R-L432F-L434W-S436N | + |
| K394R-C430S-L432F-L434W-S436N | + |
| K394R-C430P-L432F-L434W-S436N | + |
| N388M-K394R-C430S-L432F-L434W-S436A | + |
| N388M-K394R-C430P-L432F-L434W-S436A | + |
| N388M-K394R-C430P-L432F-L434W-S436N | + |
| N388M-K394R-C430S-L432F-L434W-S436N | + |
| H141Y-N388M-K394R-C430P-L432F-L434W-S436N | + |
| N388M-K394R-C430K-L432F-L434W-S436N | + |
| N388M-K394R-C430N-L432F-L434W-S436N | + |
| N388M-K394R-I395V-C430P-L432F-L434W-S436N | + |
| N388M-C430A-L432F-L434W-S436N | + |
| N388M-K394A-C430P-L432F-L434W-S436N | + |
| N388M-K394C-C430P-L432F-L434W-S436N | + |
| N388M-K394H-C430P-L432F-L434W-S436N | + |
| N388Q-C430P-L432F-L434W-S436N | + |
| S185T-N388M-C430P-L432F-L434W-S436N | + |
| N388M-K394H-C430S-L432F-L434W-S436N | + |
| S185T-N388M-C430S-L432F-L434W-S436N | + |
| N388M-K394R-C430A-L432F-L434W-S436N | + |
| N388M-K394R-C430N-L432F-L434W-S436N-H444L | + |
| N388M-K394R-C430N-L432F-L434W-S436N-H444N | + |
| N388M-K394R-C430N-L432F-L434W-S436N-H444R | + |
| N388M-K394R-C430N-L432F-L434W-S436N-H444T | + |
| N388M-K394R-C430N-L432F-L434W-S436N-H444V | + |
| N285M-N388M-K394R-C430N-L432F-L434W-S436N | + |
| N287I-N388M-K394R-C430N-L432F-L434W-S436N | + |
| N287K-N388M-K394R-C430N-L432F-L434W-S436N | + |
| N287Q-N388M-K394R-C430N-L432F-L434W-S436N | + |
| N287R-N388M-K394R-C430N-L432F-L434W-S436N | + |
| N287T-N388M-K394R-C430N-L432F-L434W-S436N | + |
| N287V-N388M-K394R-C430N-L432F-L434W-S436N | + |
| N332H-N388M-K394R-C430N-L432F-L434W-S436N | + |
| P284E-N388M-K394R-C430N-L432F-L434W-S436N | + |
| S322T-N388M-K394R-C430N-L432F-L434W-S436N | + |
| N388M-K394R-S429D-C430N-L432F-L434W-S436N | + |
| V382A-N388M-K394R-C430N-L432F-L434W-S436N | + |
| V382L-N388M-K394R-C430N-L432F-L434W-S436N | + |
| P284E-N285M-N388M-K394R-C430N-L432F-L434W-S436N | ++ |
| P284E-N287Q-N388M-K394R-C430N-L432F-L434W-S436N | ++ |
| P284E-N287V-N388M-K394R-C430N-L432F-L434W-S436N | ++ |
| P284E-N287R-N388M-K394R-C430N-L432F-L434W-S436N | ++ |
| P284E-N287T-N388M-K394R-C430N-L432F-L434W-S436N | ++ |
| P284E-N287K-N388M-K394R-C430N-L432F-L434W-S436N | ++ |
| P284E-S322T-N388M-K394R-C430N-L432F-L434W-S436N | ++ |
| N285M-N287Q-N388M-K394R-C430N-L432F-L434W-S436N | ++ |
| N285M-N287V-N388M-K394R-C430N-L432F-L434W-S436N | ++ |
| N285M-N287R-N388M-K394R-C430N-L432F-L434W-S436N | ++ |
| N285M-N287T-N388M-K394R-C430N-L432F-L434W-S436N | ++ |
| N285M-N287K-N388M-K394R-C430N-L432F-L434W-S436N | ++ |
| N285M-S322T-N388M-K394R-C430N-L432F-L434W-S436N | ++ |
| N287Q-S322T-N388M-K394R-C430N-L432F-L434W-S436N | ++ |
| N287V-S322T-N388M-K394R-C430N-L432F-L434W-S436N | ++ |
| N287R-S322T-N388M-K394R-C430N-L432F-L434W-S436N | ++ |
| N287T-S322T-N388M-K394R-C430N-L432F-L434W-S436N | ++ |
| N287K-S322T-N388M-K394R-C430N-L432F-L434W-S436N | ++ |
| P284E-N285M-N287Q-N388M-K394R-C430N-L432F-L434W-S436N | ++ |
| P284E-N285M-N287V-N388M-K394R-C430N-L432F-L434W-S436N | ++ |
| P284E-N285M-N287Q-S322T-N388M-K394R-C430N-L432F-L434W-S436N | ++ |
| P284E-N285M-N287T-N388M-K394R-C430N-L432F-L434W-S436N | ++ |
| P284E-N285M-N287K-N388M-K394R-C430N-L432F-L434W-S436N | ++ |
| P284E-N285M-S322T-N388M-K394R-C430N-L432F-L434W-S436N | ++ |
| P284E-N287Q-S322T-N388M-K394R-C430N-L432F-L434W-S436N | ++ |
| P284E-N287V-S322T-N388M-K394R-C430N-L432F-L434W-S436N | ++ |
| P284E-N287R-S322T-N388M-K394R-C430N-L432F-L434W-S436N | ++ |
| P284E-N287T-S322T-N388M-K394R-C430N-L432F-L434W-S436N | ++ |
| P284E-N287K-S322T-N388M-K394R-C430N-L432F-L434W-S436N | ++ |
| N285M-N287Q-S322T-N388M-K394R-C430N-L432F-L434W-S436N | ++ |
| N285M-N287V-S322T-N388M-K394R-C430N-L432F-L434W-S436N | ++ |
| N285M-N287R-S322T-N388M-K394R-C430N-L432F-L434W-S436N | ++ |
| N285M-N287T-S322T-N388M-K394R-C430N-L432F-L434W-S436N | ++ |
| N285M-N287K-S322T-N388M-K394R-C430N-L432F-L434W-S436N | ++ |
| P284E-N285M-N287R-N388M-K394R-C430N-L432F-L434W-S436N | ++ |
| P284E-N285M-N287V-S322T-N388M-K394R-C430N-L432F-L434W-S436N | ++ |
| P284E-N285M-N287R-S322T-N388M-K394R-C430N-L432F-L434W-S436N | ++ |
| P284E-N285M-N287T-S322T-N388M-K394R-C430N-L432F-L434W-S436N | ++ |
| P284E-N285M-N287K-S322T-N388M-K394R-C430N-L432F-L434W-S436N | ++ |
| I283T-N285P-N287T-Q290E-S322T-N388M-K394R-C430A-L432F-L434W-S436N-dG462-dA463 | ++ |
| S189C-P284E-N287R-S322T-N388M-K394R-C430N-L432F-L434W-S436N | +++ |
| I283S-P284E-N287R-S322T-N388M-K394R-C430N-L432F-L434W-S436N | +++ |
| I283F-P284E-N287R-S322T-N388M-K394R-C430N-L432F-L434W-S436N | +++ |
| I283H-P284E-N287R-S322T-N388M-K394R-C430N-L432F-L434W-S436N | +++ |
| I283V-P284E-N287R-S322T-N388M-K394R-C430N-L432F-L434W-S436N | +++ |
| P284D-N287R-S322T-N388M-K394R-C430N-L432F-L434W-S436N | +++ |
| I283T-P284E-N287R-S322T-N388M-K394R-C430N-L432F-L434W-S436N | +++ |
| I283L-P284E-N287R-S322T-N388M-K394R-C430N-L432F-L434W-S436N | +++ |
| P284E-N285T-N287R-S322T-N388M-K394R-C430N-L432F-L434W-S436N | +++ |
| P284E-E286W-N287R-S322T-N388M-K394R-C430N-L432F-L434W-S436N | +++ |
| P284E-N287S-S322T-N388M-K394R-C430N-L432F-L434W-S436N | +++ |
| P284E-N285H-N287R-S322T-N388M-K394R-C430N-L432F-L434W-S436N | +++ |
| P284E-N287P-S322T-N388M-K394R-C430N-L432F-L434W-S436N | +++ |
| P284E-N287R-S288N-S322T-N388M-K394R-C430N-L432F-L434W-S436N | +++ |
| P284E-N287R-C321V-S322T-N388M-K394R-C430N-L432F-L434W-S436N | +++ |
| P284E-N287R-S322T-A329S-N388M-K394R-C430N-L432F-L434W-S436N | +++ |
| L6M-P284E-N287R-S322T-N388M-K394R-C430N-L432F-L434W-S436N | +++ |
| P284E-N287R-S322T-N338G-N388M-K394R-C430N-L432F-L434W-S436N | +++ |
| P284E-N287R-S322T-V379I-N388M-K394R-C430N-L432F-L434W-S436N | +++ |
| P284E-N287R-S322T-V382L-N388M-K394R-C430N-L432F-L434W-S436N | +++ |
| P284E-N287R-S322T-N388Q-K394R-C430N-L432F-L434W-S436N | +++ |
| P284E-N287R-S322T-N388M-K394R-F426V-C430N-L432F-L434W-S436N | +++ |
| P284E-N287R-S322T-N388M-K394R-F426Y-C430N-L432F-L434W-S436N | +++ |
| P284E-N287R-S322T-N388M-K394R-F426W-C430N-L432F-L434W-S436N | +++ |
| V46I-P284E-N287R-S322T-N388M-K394R-C430N-L432F-L434W-S436N | +++ |
| P284E-N287R-S322T-N388M-K394R-C430N-L432F-L434W-S436W | +++ |
| P284E-N287R-S322T-N388M-K394R-C430N-L432F-L434W-S436N-C437M | +++ |
| P284E-N287R-S322T-N388M-K394R-C430N-L432F-L434W-S436N-E441D | +++ |
| P284E-N287R-S322T-N388M-K394R-C430N-L432F-L434W-S436N-E441T | +++ |
| P284E-N287R-S322T-N388M-K394R-C430N-L432F-L434W-S436N-E441H | +++ |
| P284E-N287R-S322T-N388M-K394R-C430N-L432F-L434W-S436N-H444L | +++ |
| P284E-N287R-S322T-N388M-K394R-C430N-L432F-L434W-S436N-K447W | +++ |
| P284E-N287R-S322T-N388M-K394R-C430N-L432F-L434W-S436N-K447F | +++ |
| F183Y-N285M-N287T-S322T-N388M-K394R-C430N-L432F-L434W-S436N | +++ |
| N285M-N287T-S322T-V333M-N388M-K394R-C430N-L432F-L434W-S436N | +++ |
| I283T-N285M-N287T-S322T-N388M-K394R-C430N-L432F-L434W-S436N | +++ |
| I283V-N285M-N287T-S322T-N388M-K394R-C430N-L432F-L434W-S436N | +++ |
| P284D-N285M-N287T-S322T-N388M-K394R-C430N-L432F-L434W-S436N | +++ |
| N285P-N287T-S322T-N388M-K394R-C430N-L432F-L434W-S436N | +++ |
| N285M-N287T-Q290E-S322T-N388M-K394R-C430N-L432F-L434W-S436N | +++ |
| N285M-N287T-I292L-S322T-N388M-K394R-C430N-L432F-L434W-S436N | +++ |
| N285M-N287T-I292V-S322T-N388M-K394R-C430N-L432F-L434W-S436N | +++ |
| N51D-N285M-N287T-S322T-N388M-K394R-C430N-L432F-L434W-S436N | +++ |
| N285M-N287T-C321T-S322T-N388M-K394R-C430N-L432F-L434W-S436N | +++ |
| N285M-N287T-C321V-S322T-N388M-K394R-C430N-L432F-L434W-S436N | +++ |
| N285M-N287T-S322T-N332R-N388M-K394R-C430N-L432F-L434W-S436N | +++ |
| N285M-N287T-S322T-N332S-N388M-K394R-C430N-L432F-L434W-S436N | +++ |
| N285M-N287T-S322T-T366A-Q385W-N388M-K394R-C430N-L432F-L434W-S436N | +++ |
| N285M-N287T-S322T-Q385L-N388M-K394R-C430N-L432F-L434W-S436N | +++ |
| N285M-N287T-S322T-Q385P-N388M-K394R-C430N-L432F-L434W-S436N | +++ |
| N285M-N287T-S322T-Q385S-N388M-K394R-C430N-L432F-L434W-S436N | +++ |
| N285M-N287T-S322T-N388M-S389A-K394R-C430N-L432F-L434W-S436N | +++ |
| N285M-N287T-S322T-N388M-S389Q-K394R-C430N-L432F-L434W-S436N | +++ |
| N285M-N287T-S322T-N388M-L392Y-K394R-C430N-L432F-L434W-S436N | +++ |
| R7C-N285M-N287T-S322T-N388M-K394Q-C430N-L432F-L434W-S436N | +++ |
| N285M-N287T-S322T-N388M-K394H-C430N-L432F-L434W-S436N | +++ |
| N285M-N287T-S322T-N388M-K394L-C430N-L432F-L434W-S436N | +++ |
| N285M-N287T-S322T-N388M-K394R-F426H-C430N-L432F-L434W-S436N | +++ |
| N285M-N287T-S322T-N388M-K394R-F426I-C430N-L432F-L434W-S436N | +++ |
| N285M-N287T-S322T-N388M-K394R-F426L-C430N-L432F-L434W-S436N | +++ |
| N285M-N287T-S322T-N388M-K394R-F426T-C430N-L432F-L434W-S436N | +++ |
| N285M-N287T-S322T-N388M-K394R-F426V-C430N-L432F-L434W-S436N | +++ |
| N285M-N287T-S322T-N388M-K394R-F426W-C430N-L432F-L434W-S436N | +++ |
| N285M-N287T-S322T-N388M-K394R-C430A-L432F-L434W-S436N | +++ |
| N285M-N287T-S322T-N388M-K394R-C430N-L431P-L432F-L434W-S436N | +++ |
| N285M-N287T-S322T-N388M-K394R-C430N-L432F-L434W-S436N-C437L | +++ |
| N285M-N287T-S322T-N388M-K394R-C430N-L432F-L434W-S436N-C437T | +++ |
| N285M-N287T-S322T-N388M-K394R-C430N-L432F-L434W-S436N-C437V | +++ |
| N285M-N287T-S322T-N388M-K394R-C430N-L432F-L434W-S436N-E441G | +++ |
| N285M-N287T-S322T-N388M-K394R-C430N-L432F-L434W-S436N-E441I | +++ |
| N285M-N287T-S322T-N388M-K394R-C430N-L432F-L434W-S436N-E441L | +++ |
| N285M-N287T-S322T-N388M-K394R-C430N-L432F-L434W-S436N-E441P | +++ |
| N285M-N287T-S322T-N388M-K394R-C430N-L432F-L434W-S436N-R443H | +++ |
| N285M-N287T-S322T-N388M-K394R-C430N-L432F-L434W-S436N-H444F | +++ |
| N285M-N287T-S322T-N388M-K394R-C430N-L432F-L434W-S436N-H444I | +++ |
| N285M-N287T-S322T-N388M-K394R-C430N-L432F-L434W-S436N-H444L | +++ |
| N285M-N287T-S322T-N388M-K394R-C430N-L432F-L434W-S436N-H444M | +++ |
| N285M-N287T-S322T-N388M-K394R-C430N-L432F-L434W-S436N-H444V | +++ |
| N285M-N287T-S322T-N388M-K394R-C430N-L432F-L434W-S436N-N445A | +++ |
| N285M-N287T-S322T-N388M-K394R-C430N-L432F-L434W-S436N-N445Q | +++ |
| N285M-N287T-S322T-N388M-K394R-C430N-L432F-L434W-S436N-N445T | +++ |
| N285M-N287T-S322T-N388M-K394R-C430N-L432F-L434W-S436N-R446D | +++ |
| N285M-N287T-S322T-N388M-K394R-C430N-L432F-L434W-S436N-R446G | +++ |
| N285M-N287T-S322T-N388M-K394R-C430N-L432F-L434W-S436N-R446P | +++ |
| N285M-N287T-S322T-N388M-K394R-C430N-L432F-L434W-S436N-K449W | +++ |
| N285M-N287T-S322T-N388M-K394R-C430N-L432F-L434W-S436N-K449N | +++ |
| E72K-P284E-N287Q-S322T-N388M-K394R-C430N-L432F-L434W-S436N | +++ |
| P284E-N287Q-S322T-N388M-K394R-C430N-L432F-L434W-S436N-K447H | +++ |
| P284E-N287Q-S322T-N332S-N388M-K394R-C430N-L432F-L434W-S436N | +++ |
| P284E-N287Q-S322T-N388M-K394R-C430N-L432F-L434W-S436M | +++ |
| P284E-N287Q-S322T-N388M-K394R-C430N-L432F-L434W-S436W | +++ |
| P284E-N287Q-S322T-T336H-N388M-K394R-C430N-L432F-L434W-S436N | +++ |
| P284E-N287Q-S322T-T336N-N388M-K394R-C430N-L432F-L434W-S436N | +++ |
| P284E-N287Q-S322T-T336Q-N388M-K394R-C430N-L432F-L434W-S436N | +++ |
| S89N-P284E-N287Q-S322T-A327S-N388M-K394R-C430N-L432F-L434W-S436N | +++ |
| I283C-P284E-N287Q-S322T-N388M-K394R-C430N-L432F-L434W-S436N | +++ |
| I283H-P284E-N287Q-S322T-N388M-K394R-C430N-L432F-L434W-S436N | +++ |
| I283S-P284E-N287Q-S322T-N388M-K394R-C430N-L432F-L434W-S436N | +++ |
| I283T-P284E-N287Q-S322T-N388M-K394R-C430N-L432F-L434W-S436N | +++ |
| I283V-P284E-N287Q-S322T-N388M-K394R-C430N-L432F-L434W-S436N | +++ |
| I283V-P284D-N287Q-S322T-N388M-K394R-C430N-L432F-L434W-S436N | +++ |
| P284E-N287Q-S322T-Q331G-N388M-K394R-C430N-L432F-L434W-S436N | +++ |
| P284E-N287Q-S322T-Q385F-N388M-K394R-C430N-L432F-L434W-S436N | +++ |
| P284E-N287Q-S322T-Q385P-N388M-K394R-C430N-L432F-L434W-S436N | +++ |
| P284E-N287Q-S322T-Q385R-N388M-K394R-C430N-L432F-L434W-S436N | +++ |
| P284E-N287Q-S322T-H384S-N388M-K394R-C430N-L432F-L434W-S436N | +++ |
| P284E-N287Q-S322T-N388M-K394G-C430N-L432F-L434W-S436N | +++ |
| P284E-N287Q-S322T-N388M-C430N-L432F-L434W-S436N | +++ |
| P284E-N287Q-S322T-N388M-K394L-C430N-L432F-L434W-S436N | +++ |
| R7C-P284E-N287Q-S322T-N388M-K394R-C430N-L432F-L434W-S436N | +++ |
| L48M-P284E-N287Q-S322T-N388M-K394R-C430N-L432F-L434W-S436N | +++ |
| P284E-N287Q-S322T-N388M-K394R-C430N-L432F-L434W-S436N-E440D | +++ |
| P284E-N287Q-S322T-N388M-K394R-C430N-L432F-L434W-S436N-E440I | +++ |
| P284E-N287Q-S322T-N388M-K394R-C430N-L432F-L434W-S436N-E441L | +++ |
| P284E-N287Q-S322T-N388M-K394R-C430N-L432F-L434W-S436N-E441T | +++ |
| P284E-N287Q-S322T-N388M-K394R-E428M-C430N-L432F-L434W-S436N | +++ |
| P284E-N287Q-S322T-N388M-K394R-E428T-C430N-L432F-L434W-S436N | +++ |
| P284E-N287Q-S322T-A327S-N388M-K394R-C430N-L432F-L434W-S436N | +++ |
| S189C-P284E-N287Q-S322T-N388M-K394R-C430N-L432F-L434W-S436N | +++ |
| P284E-N287Q-C321V-S322T-N388M-K394R-C430N-L432F-L434W-S436N | +++ |
| P284E-N287Q-S322T-N388M-K394R-F426L-C430N-L432F-L434W-S436N | +++ |
| P284E-N287Q-S322T-N388M-K394R-F426Y-C430N-L432F-L434W-S436N | +++ |
| N285M-N287T-Q290E-S322T-N388M-K394R-C430A-L432F-L434W-S436N | +++ |
| I283T-N285M-N287T-Q290E-S322T-N388M-K394R-C430A-L432F-L434W-S436N | +++ |
| N285P-N287T-Q290E-S322T-N388M-K394R-C430A-L432F-L434W-S436N | +++ |
| I283T-N285P-N287T-Q290E-S322T-N388M-K394R-C430A-L432F-L434W-S436N | +++ |
| P284D-N285P-N287T-Q290E-S322T-N388M-K394R-C430A-L432F-L434W-S436N | +++ |
| I283T-N285P-N287T-S322T-N388M-K394R-C430N-L432F-L434W-S436N | +++ |
| I283T-N285M-N287T-Q290E-S322T-N388M-K394R-C430N-L432F-L434W-S436N | +++ |
| I283T-N285M-N287T-S322T-N388M-K394R-C430A-L432F-L434W-S436N | +++ |
| P284D-N285P-N287T-S322T-N388M-K394R-C430N-L432F-L434W-S436N | +++ |
| I283T-N285P-N287T-S322T-N388M-K394R-C430A-L432F-L434W-S436N | +++ |
| P284D-N285P-N287T-Q290E-S322T-N388M-K394R-C430N-L432F-L434W-S436N | +++ |
| P284D-N285P-N287T-S322T-N388M-K394R-C430A-L432F-L434W-S436N | +++ |
| I283T-P284D-N285M-N287T-S322T-N388M-K394R-C430N-L432F-L434W-S436N | +++ |
| I283T-P284D-N285P-N287T-S322T-N388M-K394R-C430N-L432F-L434W-S436N | +++ |
| I283T-P284D-N285M-N287T-Q290E-S322T-N388M-K394R-C430N-L432F-L434W-S436N | +++ |
| I283T-P284D-N285M-N287T-S322T-N388M-K394R-C430A-L432F-L434W-S436N | +++ |
| I283T-P284D-N285P-N287T-Q290E-S322T-N388M-K394R-C430N-L432F-L434W-S436N | +++ |
| I283T-P284D-N285P-N287T-S322T-N388M-K394R-C430A-L432F-L434W-S436N | +++ |
| I283T-P284D-N285M-N287T-Q290E-S322T-N388M-K394R-C430A-L432F-L434W-S436N | +++ |
| I283T-P284D-N285P-N287T-Q290E-S322T-N388M-K394R-C430A-L432F-L434W-S436N | +++ |
| P284D-N285M-N287T-Q290E-S322T-N388M-K394R-C430N-L432F-L434W-S436N | +++ |
| P284D-N285M-N287T-S322T-N388M-K394R-C430A-L432F-L434W-S436N | +++ |
| N285P-N287T-Q290E-S322T-N388M-K394R-C430N-L432F-L434W-S436N | +++ |
| N285P-N287T-S322T-N388M-K394R-C430A-L432F-L434W-S436N | +++ |
| I283T-N285P-N287T-Q290E-S322T-N388M-K394R-C430N-L432F-L434W-S436N | +++ |
| I283T-P284D-N287R-S322T-N388M-K394R-C430N-L432F-L434W-S436N | +++ |
| I283T-P284D-N287P-S322T-N388M-K394R-C430N-L432F-L434W-S436N | +++ |
| I283T-P284D-N287R-S322T-N388M-C430N-L432F-L434W-S436N | +++ |
| I283T-P284D-E286W-N287P-S322T-N388M-K394R-C430N-L432F-L434W-S436N | +++ |
| I283T-P284D-N287P-S322T-N388M-C430N-L432F-L434W-S436N | +++ |
| I283C-P284E-E286W-N287P-S322T-N388M-K394R-C430N-L432F-L434W-S436N | +++ |
| I283T-P284E-E286W-N287P-S322T-N388M-K394R-C430N-L432F-L434W-S436N | +++ |
| I283C-P284E-E286W-N287P-S322T-N388M-C430N-L432F-L434W-S436N | +++ |
| I283T-P284E-E286W-N287P-S322T-N388M-C430N-L432F-L434W-S436N | +++ |
| I283T-P284D-E286W-N287P-S322T-N388M-C430N-L432F-L434W-S436N | +++ |
| I283C-P284E-N287R-S322T-N388M-K394R-C430N-L432F-L434W-S436N | +++ |
| I283C-P284D-N287P-S322T-N388M-K394R-C430N-L432F-L434W-S436N | +++ |
| I283C-P284D-N287R-S322T-N388M-C430N-L432F-L434W-S436N | +++ |
| I283C-P284D-N287P-S322T-N388M-C430N-L432F-L434W-S436N | +++ |
| I283C-P284E-N287R-S322T-N388M-C430N-L432F-L434W-S436N | +++ |
| I283T-P284E-N287R-S322T-N388M-C430N-L432F-L434W-S436N | +++ |
| P284D-N287R-S322T-N388M-C430N-L432F-L434W-S436N | +++ |
| I283C-P284E-E286W-N287R-S322T-N388M-C430N-L432F-L434W-S436N | +++ |
| I283C-P284E-N287P-S322T-N388M-C430N-L432F-L434W-S436N | +++ |
| I283T-P284E-N287P-S322T-N388M-C430N-L432F-L434W-S436N | +++ |
| I283T-P284E-E286W-N287R-S322T-N388M-K394R-C430N-L432F-L434W-S436N | +++ |
| I283T-P284E-N287P-S322T-N388M-K394R-C430N-L432F-L434W-S436N | +++ |
| P284E-E286W-N287R-S322T-N388M-C430N-L432F-L434W-S436N | +++ |
| P284E-N287P-S322T-N388M-C430N-L432F-L434W-S436N | +++ |
| I283T-P284E-E286W-N287R-S322T-N388M-C430N-L432F-L434W-S436N | +++ |
| P284D-N287P-S322T-N388M-C430N-L432F-L434W-S436N | +++ |
| I283C-P284E-E286W-N287R-S322T-N388M-K394R-C430N-L432F-L434W-S436N | +++ |
| I283C-P284E-N287P-S322T-N388M-K394R-C430N-L432F-L434W-S436N | +++ |
| P284D-N287P-S322T-N388M-K394R-C430N-L432F-L434W-S436N | +++ |
| I283C-P284D-N287Q-S322T-N388M-K394R-C430N-L432F-L434W-S436N | +++ |
| I283C-P284E-N287Q-S322T-N388M-C430N-L432F-L434W-S436N | +++ |
| I283C-P284D-N287Q-S322T-N388M-C430N-L432F-L434W-S436N | +++ |
| I283C-P284E-E286W-N287Q-S322T-N388M-C430N-L432F-L434W-S436N | +++ |
| I283T-P284D-N287Q-S322T-N388M-K394R-C430N-L432F-L434W-S436N | +++ |
| I283T-P284E-E286W-N287Q-S322T-N388M-K394R-C430N-L432F-L434W-S436N | +++ |
| I283T-P284E-N287Q-S322T-N388M-C430N-L432F-L434W-S436N | +++ |
| I283T-P284D-N287Q-S322T-N388M-C430N-L432F-L434W-S436N | +++ |
| I283T-P284E-E286W-N287Q-S322T-N388M-C430N-L432F-L434W-S436N | +++ |
| P284D-N287Q-S322T-N388M-C430N-L432F-L434W-S436N | +++ |
| P284E-E286W-N287Q-S322T-N388M-C430N-L432F-L434W-S436N | +++ |
| N285M-N287T-S322T-N388M-K394R-C430N-L432F-L434W-S436N-P458E | +++ |
| N285M-N287T-S322T-N388M-K394R-C430N-L432F-L434W-S436N-G462V | +++ |
| N285M-N287T-S322T-N388M-K394R-C430N-L432F-L434W-S436N-E459P | +++ |
| N285M-N287T-S322T-N388M-K394R-C430N-L432F-L434W-S436N-E459R | +++ |
| N285M-N287T-S322T-N388M-K394R-C430N-L432F-L434W-S436N-H461A | +++ |
| N285M-N287T-S322T-N388M-K394R-C430N-L432F-L434W-S436N-H461D | +++ |
| N285M-N287T-S322T-N388M-K394R-C430N-L432F-L434W-S436N-H461E | +++ |
| N285M-N287T-S322T-N388M-K394R-C430N-L432F-L434W-S436N-H461G | +++ |
| N285M-N287T-S322T-N388M-K394R-C430N-L432F-L434W-S436N-H461N | +++ |
| P45Q-N285M-N287T-S322T-N388M-K394R-C430N-L432F-L434W-S436N-H461S | +++ |
| N285M-N287T-S322T-N388M-K394R-C430N-L432F-L434W-S436N-G462A | +++ |
| N285M-N287T-S322T-N388M-K394R-C430N-L432F-L434W-S436N-G462C | +++ |
| N285M-N287T-S322T-N388M-K394R-C430N-L432F-L434W-S436N-G462P | +++ |
| N285M-N287T-S322T-N388M-K394R-C430N-L432F-L434W-S436N-G462I | +++ |
| N285M-N287T-S322T-N388M-K394R-C430N-L432F-L434W-S436N-A463L | +++ |
| N285M-N287T-S322T-N388M-K394R-C430N-L432F-L434W-S436N-A463P | +++ |
| N285M-N287T-S322T-N388M-K394R-C430N-L432F-L434W-S436N-Y469D | +++ |
| N285M-N287T-S322T-N388M-K394R-C430N-L432F-L434W-S436N-Y469E | +++ |
| N285M-N287T-S322T-N388M-K394R-C430N-L432F-L434W-S436N-Y469S | +++ |
| N285M-N287T-S322T-N388M-K394R-C430N-L432F-L434W-S436N-Y469T | +++ |
| N285M-N287T-S322T-N388M-K394R-C430N-L432F-L434W-S436N-Y471F | +++ |
| H141L-N285M-N287T-S322T-N388M-K394R-C430N-L432F-L434W-S436N | +++ |
| N144S-N285M-N287T-S322T-N388M-K394R-C430N-L432F-L434W-S436N | +++ |
| R15H-N285M-N287T-S322T-N388M-K394R-C430N-L432F-L434W-S436N | +++ |
| P284E-N287Q-S322T-N388M-K394R-C430N-L432F-L434W-S436N-E459L | +++ |
| V46I-P284E-N287Q-S322T-N388M-K394R-C430N-L432F-L434W-S436N-N460V | +++ |
| P284E-N287Q-S322T-N388M-K394R-C430N-L432F-L434W-S436N-N460G | +++ |
| P284E-N287Q-S322T-N388M-K394R-C430N-L432F-L434W-S436N-N460I | +++ |
| P284E-N287Q-S322T-N388M-K394R-C430N-L432F-L434W-S436N-H461A | +++ |
| P284E-N287Q-S322T-N388M-K394R-C430N-L432F-L434W-S436N-H461G | +++ |
| P284E-N287Q-S322T-N388M-K394R-C430N-L432F-L434W-S436N-H461N | +++ |
| P284E-N287Q-S322T-N388M-K394R-C430N-L432F-L434W-S436N-H461T | +++ |
| P284E-N287Q-S322T-N388M-K394R-C430N-L432F-L434W-S436N-H461D | +++ |
| P284E-N287Q-S322T-N388M-K394R-C430N-L432F-L434W-S436N-A463E | +++ |
| P284E-N287Q-S322T-N388M-K394R-C430N-L432F-L434W-S436N-A463G | +++ |
| P284E-N287Q-S322T-N388M-K394R-C430N-L432F-L434W-S436N-F467W | +++ |
| P284E-N287Q-S322T-N388M-K394R-C430N-L432F-L434W-S436N-Y469D | +++ |
| P284E-N287Q-S322T-N388M-K394R-C430N-L432F-L434W-S436N-Y469N | +++ |
| P284E-N287Q-S322T-N388M-K394R-C430N-L432F-L434W-S436N-Y469S | +++ |
| P284E-N287Q-S322T-N388M-K394R-C430N-L432F-L434W-S436N-Y469T | +++ |
| P284E-N287Q-S322T-N388M-K394R-C430N-L432F-L434W-S436N-A470G | +++ |
| P284E-N287Q-S322T-N388M-K394R-C430N-L432F-L434W-S436N-Y471F | +++ |
| H141Q-P284E-N287Q-S322T-N388M-K394R-C430N-L432F-L434W-S436N | +++ |
| H141S-P284E-N287Q-S322T-N388M-K394R-C430N-L432F-L434W-S436N | +++ |
| H141Y-P284E-N287Q-S322T-N388M-K394R-C430N-L432F-L434W-S436N | +++ |
| N285M-N287T-S322T-N388M-K394R-C430N-L432F-L434W-S436N-dP458 | +++ |
| N285M-N287T-S322T-N388M-K394R-C430N-L432F-L434W-S436N-dE459 | +++ |
| N285M-N287T-S322T-N388M-K394R-C430N-L432F-L434W-S436N-dN460 | +++ |
| N285M-N287T-S322T-N388M-K394R-C430N-L432F-L434W-S436N-dH461 | +++ |
| N285M-N287T-S322T-N388M-K394R-C430N-L432F-L434W-S436N-dG462 | +++ |
| N285M-N287T-S322T-N388M-K394R-C430N-L432F-L434W-S436N-dA463 | +++ |
| P284E-N287Q-S322T-N388M-K394R-C430N-L432F-L434W-S436N-dP458 | +++ |
| P284E-N287Q-S322T-N388M-K394R-C430N-L432F-L434W-S436N-dE459 | +++ |
| P284E-N287Q-S322T-N388M-K394R-C430N-L432F-L434W-S436N-dN460 | +++ |
| P284E-N287Q-S322T-N388M-K394R-C430N-L432F-L434W-S436N-dH461 | +++ |
| P284E-N287Q-S322T-N388M-K394R-C430N-L432F-L434W-S436N-dG462 | +++ |
| P284E-N287Q-S322T-N388M-K394R-C430N-L432F-L434W-S436N-dA463 | +++ |
| P284E-N287Q-S322T-N388M-K394R-C430N-L432F-L434W-S436N-H461G-Y469D | +++ |
| P284E-N287Q-S322T-N388M-K394R-C430N-L432F-L434W-S436N-A463G-Y469D | +++ |
| P284E-N287Q-S322T-N388M-K394R-C430N-L432F-L434W-S436N-H461G-A463G-Y469D | +++ |
| P284E-N287Q-S322T-N388M-K394R-C430N-L432F-L434W-S436N-N460G-H461G-A463G-Y469D | +++ |
| P284D-N287P-S322T-N388M-C430N-L432F-L434W-S436N-N460G | +++ |
| P284D-N287P-S322T-N388M-C430N-L432F-L434W-S436N-N460I | +++ |
| P284D-N287P-S322T-N388M-C430N-L432F-L434W-S436N-H461G | +++ |
| P284D-N287P-S322T-N388M-C430N-L432F-L434W-S436N-A463G | +++ |
| P284D-N287P-S322T-N388M-C430N-L432F-L434W-S436N-Y469D | +++ |
| P284D-N287P-S322T-N388M-C430N-L432F-L434W-S436N-N460G-H461G | +++ |
| P284D-N287P-S322T-N388M-C430N-L432F-L434W-S436N-N460G-A463G | +++ |
| P284D-N287P-S322T-N388M-C430N-L432F-L434W-S436N-N460G-Y469D | +++ |
| P284D-N287P-S322T-N388M-C430N-L432F-L434W-S436N-N460I-A463G | +++ |
| P284D-N287P-S322T-N388M-C430N-L432F-L434W-S436N-N460I-Y469D | +++ |
| P284D-N287P-S322T-N388M-C430N-L432F-L434W-S436N-H461G-A463G | +++ |
| P284D-N287P-S322T-N388M-C430N-L432F-L434W-S436N-H461G-Y469D | +++ |
| P284D-N287P-S322T-N388M-C430N-L432F-L434W-S436N-A463G-Y469D | +++ |
| P284D-N287P-S322T-N388M-C430N-L432F-L434W-S436N-N460G-H461G-A463G | +++ |
| P284D-N287P-S322T-N388M-C430N-L432F-L434W-S436N-N460G-H461G-Y469D | +++ |
| P284D-N287P-S322T-N388M-C430N-L432F-L434W-S436N-N460G-A463G-Y469D | +++ |
| P284D-N287P-S322T-N388M-C430N-L432F-L434W-S436N-N460I-H461G-Y469D | +++ |
| P284D-N287P-S322T-N388M-C430N-L432F-L434W-S436N-N460I-A463G-Y469D | +++ |
| P284D-N287P-S322T-N388M-C430N-L432F-L434W-S436N-H461G-A463G-Y469D | +++ |
| P284D-N287P-S322T-N388M-C430N-L432F-L434W-S436N-N460G-H461G-A463G-Y469D | +++ |
| P284D-N287P-S322T-N388M-C430N-L432F-L434W-S436N-N460I-H461G-A463G-Y469D | +++ |
| I283T-P284D-N287P-S322T-N388M-C430N-L432F-L434W-S436N-N460G | +++ |
| I283T-P284D-N287P-S322T-N388M-C430N-L432F-L434W-S436N-A463G | +++ |
| I283T-P284D-N287P-S322T-N388M-C430N-L432F-L434W-S436N-Y469D | +++ |
| I283T-P284D-N287P-S322T-N388M-C430N-L432F-L434W-S436N-N460G-A463G | +++ |
| I283T-P284D-N287P-S322T-N388M-C430N-L432F-L434W-S436N-N460G-Y469D | +++ |
| I283T-P284D-N287P-S322T-N388M-C430N-L432F-L434W-S436N-N460G-A463G-Y469D | +++ |
| I283T-P284D-N287P-S322T-N388M-C430N-L432F-L434W-S436N-N460I-H461G-Y469D | +++ |
| I283T-P284D-N287P-S322T-N388M-C430N-L432F-L434W-S436N-N460I-A463G-Y469D | +++ |
| I283T-P284D-N287P-S322T-N388M-C430N-L432F-L434W-S436N-H461G-A463G-Y469D | +++ |
| I283T-P284D-N287P-S322T-N388M-C430N-L432F-L434W-S436N-N460G-H461G-A463G-Y469D | +++ |
| I283T-P284D-N287P-S322T-N388M-C430N-L432F-L434W-S436N-N460I-H461G | +++ |
| I283T-P284D-N287P-S322T-N388M-C430N-L432F-L434W-S436N-N460I-A463G | +++ |
| I283T-P284D-N287P-S322T-N388M-C430N-L432F-L434W-S436N-N460I-Y469D | +++ |
| I283T-P284D-N287P-S322T-N388M-C430N-L432F-L434W-S436N-H461G-A463G | +++ |
| I283T-P284D-N287P-S322T-N388M-C430N-L432F-L434W-S436N-H461G-Y469D | +++ |
| I283T-P284D-N287P-S322T-N388M-C430N-L432F-L434W-S436N-A463G-Y469D | +++ |
| I283T-P284D-N287P-S322T-N388M-C430N-L432F-L434W-S436N-N460G-H461G-A463G | +++ |
| I283T-P284D-N287P-S322T-N388M-C430N-L432F-L434W-S436N-N460G-H461G-Y469D | +++ |
| I283T-P284D-N287Q-S322T-N388M-C430N-L432F-L434W-S436N-H461G | +++ |
| I283T-P284D-N287Q-S322T-N388M-C430N-L432F-L434W-S436N-A463G | +++ |
| I283T-P284D-N287Q-S322T-N388M-C430N-L432F-L434W-S436N-Y469D | +++ |
| I283T-P284D-N287Q-S322T-N388M-C430N-L432F-L434W-S436N-N460G-H461G | +++ |
| I283T-P284D-N287Q-S322T-N388M-C430N-L432F-L434W-S436N-N460G-A463G | +++ |
| I283T-P284D-N287Q-S322T-N388M-C430N-L432F-L434W-S436N-N460G-Y469D | +++ |
| I283T-P284D-N287Q-S322T-N388M-C430N-L432F-L434W-S436N-N460I-Y469D | +++ |
| I283T-P284D-N287Q-S322T-N388M-C430N-L432F-L434W-S436N-H461G-Y469D | +++ |
| I283T-P284D-N287Q-S322T-N388M-C430N-L432F-L434W-S436N-A463G-Y469D | +++ |
| I283T-P284D-N287Q-S322T-N388M-C430N-L432F-L434W-S436N-N460G-H461G-A463G | +++ |
| I283T-P284D-N287Q-S322T-N388M-C430N-L432F-L434W-S436N-N460G-H461G-Y469D | +++ |
| I283T-P284D-N287Q-S322T-N388M-C430N-L432F-L434W-S436N-N460I-H461G-Y469D | +++ |
| I283T-P284D-N287Q-S322T-N388M-C430N-L432F-L434W-S436N-N460G-H461G-A463G-Y469D | +++ |
| P284D-N287P-S322T-N388M-C430N-L432F-L434W-S436N-dG462 | +++ |
| I283T-P284D-N287P-S322T-N388M-C430N-L432F-L434W-S436N-dG462 | +++ |
| P284D-N287P-S322T-N388M-C430N-L432F-L434W-S436N-dA463 | +++ |
| I283T-P284D-N287P-S322T-N388M-C430N-L432F-L434W-S436N-dA463 | +++ |
| I283T-P284D-N287Q-S322T-N388M-C430N-L432F-L434W-S436N-dG462 | +++ |
| I283T-P284D-N287Q-S322T-N388M-C430N-L432F-L434W-S436N-dA463 | +++ |
| I283T-P284D-N285P-N287T-S322T-N388M-K394R-C430N-L432F-L434W-S436N-G462A | +++ |
| I283T-P284D-N285P-N287T-S322T-N388M-K394R-C430N-L432F-L434W-S436N-G462P | +++ |
| I283T-P284D-N285P-N287T-S322T-N388M-K394R-C430N-L432F-L434W-S436N-G462V | +++ |
| I283T-P284D-N285P-N287T-S322T-N388M-K394R-C430N-L432F-L434W-S436N-Y469D | +++ |
| I283T-P284D-N285P-N287T-S322T-N388M-K394R-C430N-L432F-L434W-S436N-G462A-A463P | +++ |
| I283T-P284D-N285P-N287T-S322T-N388M-K394R-C430N-L432F-L434W-S436N-G462A-Y469D | +++ |
| I283T-P284D-N285P-N287T-S322T-N388M-K394R-C430N-L432F-L434W-S436N-G462P-A463P | +++ |
| I283T-P284D-N285P-N287T-S322T-N388M-K394R-C430N-L432F-L434W-S436N-G462P-Y469D | +++ |
| N285M-N287T-S322T-N388M-K394R-C430N-L432F-L434W-S436N-G462V-A463P | +++ |
| N285M-N287T-S322T-N388M-K394R-C430N-L432F-L434W-S436N-G462V-Y469D | +++ |
| N285M-N287T-S322T-N388M-K394R-C430N-L432F-L434W-S436N-A463P-Y469D | +++ |
| N285M-N287T-S322T-N388M-K394R-C430N-L432F-L434W-S436N-G462A-A463P-Y469D | +++ |
| N285M-N287T-S322T-N388M-K394R-C430N-L432F-L434W-S436N-G462P-A463P-Y469D | +++ |
| I283T-P284D-N285P-N287T-S322T-N388M-K394R-C430N-L432F-L434W-S436N-G462V-A463P | +++ |
| I283T-P284D-N285P-N287T-S322T-N388M-K394R-C430N-L432F-L434W-S436N-G462V-Y469D | +++ |
| I283T-P284D-N285P-N287T-S322T-N388M-K394R-C430N-L432F-L434W-S436N-A463P-Y469D | +++ |
| I283T-P284D-N285P-N287T-S322T-N388M-K394R-C430N-L432F-L434W-S436N-G462A-A463P-Y469D | +++ |
| I283T-P284D-N285P-N287T-S322T-N388M-K394R-C430N-L432F-L434W-S436N-G462P-A463P-Y469D | +++ |
| I283T-P284D-N285P-N287T-S322T-N388M-K394R-C430N-L432F-L434W-S436N-G462V-A463P-Y469D | +++ |
| I283T-P284D-N285M-N287T-S322T-N388M-K394R-C430A-L432F-L434W-S436N-G462A | +++ |
| I283T-P284D-N285M-N287T-S322T-N388M-K394R-C430A-L432F-L434W-S436N-G462P | +++ |
| I283T-P284D-N285M-N287T-S322T-N388M-K394R-C430A-L432F-L434W-S436N-G462V | +++ |
| I283T-P284D-N285M-N287T-S322T-N388M-K394R-C430A-L432F-L434W-S436N-Y469D | +++ |
| I283T-P284D-N285M-N287T-S322T-N388M-K394R-C430A-L432F-L434W-S436N-G462A-A463P | +++ |
| I283T-P284D-N285M-N287T-S322T-N388M-K394R-C430A-L432F-L434W-S436N-G462A-Y469D | +++ |
| I283T-P284D-N285M-N287T-S322T-N388M-K394R-C430A-L432F-L434W-S436N-G462P-A463P | +++ |
| I283T-P284D-N285M-N287T-S322T-N388M-K394R-C430A-L432F-L434W-S436N-G462P-Y469D | +++ |
| I283T-P284D-N285M-N287T-S322T-N388M-K394R-C430A-L432F-L434W-S436N-G462V-A463P | +++ |
| I283T-P284D-N285M-N287T-S322T-N388M-K394R-C430A-L432F-L434W-S436N-G462V-Y469D | +++ |
| I283T-P284D-N285M-N287T-S322T-N388M-K394R-C430A-L432F-L434W-S436N-A463P-Y469D | +++ |
| I283T-P284D-N285M-N287T-S322T-N388M-K394R-C430A-L432F-L434W-S436N-G462A-A463P-Y469D | +++ |
| I283T-P284D-N285M-N287T-S322T-N388M-K394R-C430A-L432F-L434W-S436N-G462P-A463P-Y469D | +++ |
| I283T-P284D-N285M-N287T-S322T-N388M-K394R-C430A-L432F-L434W-S436N-G462V-A463P-Y469D | +++ |
| I283T-N285M-N287T-Q290E-S322T-N388M-K394R-C430A-L432F-L434W-S436N-G462A | +++ |
| I283T-N285M-N287T-Q290E-S322T-N388M-K394R-C430A-L432F-L434W-S436N-G462P | +++ |
| I283T-N285M-N287T-Q290E-S322T-N388M-K394R-C430A-L432F-L434W-S436N-G462V | +++ |
| I283T-N285M-N287T-Q290E-S322T-N388M-K394R-C430A-L432F-L434W-S436N-A463P | +++ |
| I283T-N285M-N287T-Q290E-S322T-N388M-K394R-C430A-L432F-L434W-S436N-Y469D | +++ |
| I283T-N285M-N287T-Q290E-S322T-N388M-K394R-C430A-L432F-L434W-S436N-G462A-A463P | +++ |
| I283T-N285M-N287T-Q290E-S322T-N388M-K394R-C430A-L432F-L434W-S436N-G462A-Y469D | +++ |
| I283T-N285M-N287T-Q290E-S322T-N388M-K394R-C430A-L432F-L434W-S436N-G462P-A463P | +++ |
| I283T-N285M-N287T-Q290E-S322T-N388M-K394R-C430A-L432F-L434W-S436N-G462P-Y469D | +++ |
| I283T-N285M-N287T-Q290E-S322T-N388M-K394R-C430A-L432F-L434W-S436N-G462V-A463P | +++ |
| I283T-N285M-N287T-Q290E-S322T-N388M-K394R-C430A-L432F-L434W-S436N-G462V-Y469D | +++ |
| I283T-N285M-N287T-Q290E-S322T-N388M-K394R-C430A-L432F-L434W-S436N-A463P-Y469D | +++ |
| I283T-N285M-N287T-Q290E-S322T-N388M-K394R-C430A-L432F-L434W-S436N-G462A-A463P-Y469D | +++ |
| I283T-N285M-N287T-Q290E-S322T-N388M-K394R-C430A-L432F-L434W-S436N-G462P-A463P-Y469D | +++ |
| I283T-N285M-N287T-Q290E-S322T-N388M-K394R-C430A-L432F-L434W-S436N-G462V-A463P-Y469D | +++ |
| I283T-N285P-N287T-Q290E-S322T-N388M-K394R-C430A-L432F-L434W-S436N-G462A | +++ |
| I283T-N285P-N287T-Q290E-S322T-N388M-K394R-C430A-L432F-L434W-S436N-G462P | +++ |
| I283T-N285P-N287T-Q290E-S322T-N388M-K394R-C430A-L432F-L434W-S436N-G462V | +++ |
| I283T-N285P-N287T-Q290E-S322T-N388M-K394R-C430A-L432F-L434W-S436N-A463P | +++ |
| I283T-N285P-N287T-Q290E-S322T-N388M-K394R-C430A-L432F-L434W-S436N-Y469D | +++ |
| I283T-N285P-N287T-Q290E-S322T-N388M-K394R-C430A-L432F-L434W-S436N-G462A-A463P | +++ |
| I283T-N285P-N287T-Q290E-S322T-N388M-K394R-C430A-L432F-L434W-S436N-G462A-Y469D | +++ |
| I283T-N285P-N287T-Q290E-S322T-N388M-K394R-C430A-L432F-L434W-S436N-G462P-A463P | +++ |
| I283T-N285P-N287T-Q290E-S322T-N388M-K394R-C430A-L432F-L434W-S436N-G462P-Y469D | +++ |
| I283T-N285P-N287T-Q290E-S322T-N388M-K394R-C430A-L432F-L434W-S436N-G462V-A463P | +++ |
| I283T-N285P-N287T-Q290E-S322T-N388M-K394R-C430A-L432F-L434W-S436N-G462V-Y469D | +++ |
| I283T-N285P-N287T-Q290E-S322T-N388M-K394R-C430A-L432F-L434W-S436N-A463P-Y469D | +++ |
| I283T-N285P-N287T-Q290E-S322T-N388M-K394R-C430A-L432F-L434W-S436N-G462A-A463P-Y469D | +++ |
| I283T-N285P-N287T-Q290E-S322T-N388M-K394R-C430A-L432F-L434W-S436N-G462P-A463P-Y469D | +++ |
| I283T-N285P-N287T-Q290E-S322T-N388M-K394R-C430A-L432F-L434W-S436N-G462V-A463P-Y469D | +++ |
| N285M-N287T-S322T-N388M-K394R-C430N-L432F-L434W-S436N-G462A-A463P | +++ |
| N285M-N287T-S322T-N388M-K394R-C430N-L432F-L434W-S436N-G462A-Y469D | +++ |
| N285M-N287T-S322T-N388M-K394R-C430N-L432F-L434W-S436N-G462P-A463P | +++ |
| N285M-N287T-S322T-N388M-K394R-C430N-L432F-L434W-S436N-G462P-Y469D | +++ |
| R7C-I283T-N285P-N287T-Q290E-S322T-Q385D-N388M-K394R-C430A-L432F-L434W-S436N-dG462-dA463 | +++ |
| I283T-N285P-N287T-Q290E-S322T-N388M-K394R-i421aP-C430A-L432F-L434W-S436N-dG462-dA463 | +++ |
| P184A-I283T-N285P-N287T-Q290E-S322T-N388M-K394R-C430A-L432F-L434W-S436N-dG462-dA463 | +++ |
| I283T-N285P-N287T-Q290E-S322T-N388M-K394C-C430A-L432F-L434W-S436N-dG462-dA463 | +++ |
| I283T-N285P-N287T-Q290E-S322T-N388M-K394R-R422S-C430A-L432F-L434W-S436N-dG462-dA463 | +++ |
| I283T-N285P-N287T-Q290E-S322T-N388M-K394R-H419P-C430A-L432F-L434W-S436N-dG462-dA463 | +++ |
| I283T-N285P-N287T-Q290E-S322T-N388M-K394R-C430A-L432F-L434W-S436N-N460L-dG462-dA463 | +++ |
| I283T-N285P-N287T-Q290E-S322T-N388M-K394R-C430A-L432F-L434W-S436N-S439L-dG462-dA463 | +++ |
| I283T-N285P-N287T-Q290E-S322T-N388M-K394R-C430A-L432F-L434W-S436N-P458L-dG462-dA463 | +++ |
| I283T-N285P-N287T-Q290E-S322T-N388M-K394R-C430A-L432F-L434W-S436N-L457A-dG462-dA463 | +++ |
| I283T-N285P-N287T-Q290E-S322T-N388M-K394R-C430A-L432F-L434W-S436N-P458C-dG462-dA463 | +++ |
| I283T-N285P-N287T-Q290E-S322T-N388M-K394R-C430A-L432F-L434W-S436N-E459T-dG462-dA463 | +++ |
| I283T-N285P-N287T-Q290E-S322T-N388M-K394R-C430A-L432F-L434W-S436N-P458M-dG462-dA463 | +++ |
| I283T-N285P-N287T-Q290E-S322T-N388M-K394R-C430A-L432F-L434W-S436N-P458I-dG462-dA463 | +++ |
| I283T-N285P-N287T-Q290E-S322T-N388M-K394R-C430A-L432F-L434W-S436N-E459G-dG462-dA463 | +++ |
| I283T-N285P-N287T-Q290E-S322T-N388M-K394R-C430A-L432F-L434W-S436N-P458R-dG462-dA463 | +++ |
| I283T-N285P-N287T-Q290E-S322T-N388M-K394R-C430A-L432F-L434W-S436N-P458Q-dG462-dA463 | +++ |
| I283T-N285P-N287T-Q290E-S322T-N388M-K394R-C430A-L432F-L434W-S436N-N460H-dG462-dA463 | +++ |
| I283T-N285P-N287T-Q290E-S322T-N388M-K394R-C430A-L432F-L434W-S436N-N460Y-dG462-dA463 | +++ |
| I283T-N285P-N287T-Q290E-S322T-N388M-K394R-C430A-L432F-L434W-S436N-N460I-dG462-dA463 | +++ |
| I283T-N285P-N287T-Q290E-S322T-N388M-K394R-C430A-L432F-L434W-S436N-N460M-dG462-dA463 | +++ |
| I283T-P284D-N287P-S322T-N388M-K423C-C430N-L432F-L434W-S436N-A463G-Y469D | ++++ |
| I283T-P284D-N285A-N287P-S322T-N388M-C430N-L432F-L434W-S436N-A463G-Y469D | ++++ |
| I283T-P284D-N285I-N287P-S322T-N388M-C430N-L432F-L434W-S436N-A463G-Y469D | ++++ |
| I283T-P284D-N285L-N287P-S322T-N388M-C430N-L432F-L434W-S436N-A463G-Y469D | ++++ |
| I283T-P284D-N287P-S322T-N388M-C430N-L432F-L434W-S436T-A463G-Y469D | ++++ |
| I283F-P284D-N287P-S322T-N388M-C430N-L432F-L434W-S436N-A463G-Y469D | ++++ |
| I283S-P284D-N287P-S322T-N388M-C430N-L432F-L434W-S436N-A463G-Y469D | ++++ |
| I283T-P284D-N287P-S288T-S322T-N388M-C430N-L432F-L434W-S436N-A463G-Y469D | ++++ |
| I283T-P284D-N287P-S322T-N388M-S429A-C430N-L432F-L434W-S436N-A463G-Y469D | ++++ |
| I283T-P284D-N287P-S322T-H384A-N388M-C430N-L432F-L434W-S436N-A463G-Y469D | ++++ |
| I283T-P284D-N287P-S322T-H384T-N388M-C430N-L432F-L434W-S436N-A463G-Y469D | ++++ |
| I283T-P284D-N287P-S322T-N388M-C430N-L432F-L434W-S436N-H444R-A463G-Y469D | ++++ |
| I283T-P284D-N287P-S322T-N388M-P393A-C430N-L432F-L434W-S436N-A463G-Y469D | ++++ |
| I283T-P284D-N287P-S322T-N388M-R422F-C430N-L432F-L434W-S436N-A463G-Y469D | ++++ |
| I283T-P284D-N287P-S322T-N388M-R422Q-C430N-L432F-L434W-S436N-A463G-Y469D | ++++ |
| I283T-P284D-N287P-S322T-N388M-R422V-C430N-L432F-L434W-S436N-A463G-Y469D | ++++ |
| I283T-P284D-N287P-S322T-N388M-E421T-C430N-L432F-L434W-S436N-A463G-Y469D | ++++ |
| I283T-P284D-N287P-S322T-N388M-E421V-C430N-L432F-L434W-S436N-A463G-Y469D | ++++ |
| I283T-P284D-N287P-S322T-N388M-C430N-L432F-L434W-S436N-E459P-A463G-Y469D | ++++ |
| I283T-P284D-N287P-S322T-N388M-C430N-L432F-L434W-S436N-A463G-Y469T | ++++ |
| I283T-P284D-E286C-N287P-S322T-N388M-C430N-L432F-L434W-S436N-A463G-Y469D | ++++ |
| I283T-P284D-N287P-S322T-N388M-C430N-L432F-L434W-S436N-A463F-Y469D | ++++ |
| I283T-P284D-N287P-S322T-N388M-C430N-L432F-L434W-S436N-G462P-A463G-Y469D | ++++ |
| I283T-P284D-N287P-S322T-N388M-i421aG-C430N-L432F-L434W-S436N-A463G-Y469D | ++++ |
| I283F-P284E-N287Q-S322T-N388M-K394R-C430N-L432F-L434W-S436N-dG462 | ++++ |
| P284E-N287Q-S322T-N388M-K394R-C430N-L432F-L434W-S436N-dG462-Y469S | ++++ |
| P284E-N287Q-S322T-N388M-K394R-C430N-L432F-L434W-S436N-dG462-Y469D | ++++ |
| I283L-N285M-N287T-S322T-A337T-N388M-K394R-C430N-L432F-L434W-S436N-dG462 | ++++ |
| N285M-N287T-S322T-N388M-K394R-C430N-L432F-L434W-S436N-N460G-dG462 | ++++ |
| N285M-N287T-S322T-N388M-K394R-C430N-L432F-L434W-S436N-dG462-A463M | ++++ |
| N285M-N287T-S322T-N388M-K394R-C430N-L432F-L434W-S436N-dG462-A463F | ++++ |
| N285M-N287T-S322T-N388M-K394R-C430N-L432F-L434W-S436N-dG462-A463V | ++++ |
| N285M-N287T-S322T-N388M-K394R-C430N-L432F-L434W-S436N-dG462-Y469S | ++++ |
| N285M-N287T-S322T-N388M-K394R-C430N-L432F-L434W-S436N-dG462-Y469T | ++++ |
| N285M-N287T-S322T-N388M-K394R-C430N-L432F-L434W-S436N-dG462-Y469N | ++++ |
| N285M-N287T-S322T-N388M-K394R-C430N-L432F-L434W-S436N-dG462-A470S | ++++ |
| N285M-N287T-S322T-N388M-K394R-C430N-L432F-L434W-S436N-dA463-A470S | ++++ |
| N285M-N287T-S322T-N388M-K394R-C430N-L432F-L434W-S436N-dA463-S465I | ++++ |
| N285M-N287T-S322T-N388M-K394R-C430N-L432F-L434W-S436N-dA463-S465V | ++++ |
| I283Q-P284E-N287Q-S322T-N388M-K394R-C430N-L432F-L434W-S436N-dA463 | ++++ |
| I283C-P284E-N287Q-S322T-N388M-K394R-C430N-L432F-L434W-S436N-dA463 | ++++ |
| I283L-P284E-N287Q-S322T-N388M-K394R-C430N-L432F-L434W-S436N-dA463 | ++++ |
| P284D-N287Q-S322T-N388M-K394R-C430N-L432F-L434W-S436N-dA463 | ++++ |
| P284E-N287Q-L289H-S322T-N388M-K394R-C430N-L432F-L434W-S436N-dA463 | ++++ |
| P284E-N287Q-L289R-S322T-N388M-K394R-C430N-L432F-L434W-S436N-dA463 | ++++ |
| P284E-N287Q-S322T-N388M-K394R-R422H-C430N-L432F-L434W-S436N-dA463 | ++++ |
| P284E-N287Q-S322T-N388M-K394R-C430N-L432F-L434W-S436N-dA463-A470T | ++++ |
| P284E-N287Q-S322T-N388M-K394R-C430N-L432F-L434W-S436N-G462R-dA463 | ++++ |
| P284E-N287Q-S322T-N388M-K394R-C430N-L432F-L434W-S436N-dA463-S465A | ++++ |
| P284E-N287Q-S322T-N388M-K394R-C430N-L432F-L434W-S436N-dA463-Y469T | ++++ |
| P284E-N287Q-S322T-N388M-K394R-C430N-L432F-L434W-S436N-dA463-Y469E | ++++ |
| P284E-N287Q-S322T-N388M-K394R-C430N-L432F-L434W-S436N-dA463-A470S | ++++ |
| I283T-N285P-E286D-N287T-Q290E-S322T-N388M-K394R-C430A-L432F-L434W-S436N-G462P-A463P | ++++ |
| I283T-N285P-N287T-L289Y-Q290E-S322T-N388M-K394R-C430A-L432F-L434W-S436N-G462P-A463P | ++++ |
| I283T-N285P-N287T-L289A-Q290E-S322T-N388M-K394R-C430A-L432F-L434W-S436N-G462P-A463P | ++++ |
| I283T-N285P-N287T-Q290E-S322T-Q365M-N388M-K394R-C430A-L432F-L434W-S436N-G462P-A463P | ++++ |
| I283T-N285P-N287T-Q290E-S322T-Q365S-N388M-K394R-C430A-L432F-L434W-S436N-G462P-A463P | ++++ |
| I283L-N285P-N287T-Q290E-S322T-N388M-K394R-C430A-L432F-L434W-S436N | ++++ |
| I283H-P284D-N285M-N287T-S322T-N388M-K394R-C430A-L432F-L434W-S436N-G462P-A463P | ++++ |
| I283Q-P284D-N285M-N287T-S322T-N388M-K394R-C430A-L432F-L434W-S436N-G462P-A463P | ++++ |
| I283T-P284D-N285H-N287T-S322T-N388M-K394R-C430A-L432F-L434W-S436N-G462P-A463P | ++++ |
| I283T-P284D-N285V-N287T-S322T-N388M-K394R-C430A-L432F-L434W-S436N-G462P-A463P | ++++ |
| I283T-P284D-N285L-N287T-S322T-N388M-K394R-C430A-L432F-L434W-S436N-G462P-A463P | ++++ |
| I283T-P284D-N285T-N287T-S322T-N388M-K394R-C430A-L432F-L434W-S436N-G462P-A463P | ++++ |
| I283T-P284D-N285S-N287T-S322T-N388M-K394R-C430A-L432F-L434W-S436N-G462P-A463P | ++++ |
| I283T-P284D-N285M-E286N-N287T-S322T-N388M-K394R-C430A-L432F-L434W-S436N-G462P-A463P | ++++ |
| I283T-P284D-N285M-E286A-N287T-S322T-N388M-K394R-C430A-L432F-L434W-S436N-G462P-A463P | ++++ |
| I283T-P284D-N285M-E286G-N287T-S322T-N388M-K394R-C430A-L432F-L434W-S436N-G462P-A463P | ++++ |
| I283T-P284D-N285M-E286H-N287T-S322T-N388M-K394R-C430A-L432F-L434W-S436N-G462P-A463P | ++++ |
| I283T-P284D-N285M-E286D-N287T-S322T-N388M-K394R-C430A-L432F-L434W-S436N-G462P-A463P | ++++ |
| I283T-P284D-N285M-N287T-S288T-S322T-N388M-K394R-C430A-L432F-L434W-S436N-G462P-A463P | ++++ |
| I283T-P284D-N285M-N287T-L289Q-S322T-N388M-K394R-C430A-L432F-L434W-S436N-G462P-A463P | ++++ |
| I283T-P284D-N285M-N287T-L289R-S322T-N388M-K394R-C430A-L432F-L434W-S436N-G462P-A463P | ++++ |
| I283T-P284D-N285M-N287T-L289M-S322T-N388M-K394R-C430A-L432F-L434W-S436N-G462P-A463P | ++++ |
| I283T-P284D-N285M-N287T-S322T-N388M-K394R-C430T-L432F-L434W-S436N-G462P-A463P | ++++ |
| I283T-P284D-N287P-S322T-H384A-N388M-C430N-L432F-L434W-S436N-dG462 | ++++ |
| P25L-I283T-P284D-N287P-S322T-N388M-C430N-L432F-L434W-S436N-dG462 | ++++ |
| I283T-P284D-N287P-S322T-N388M-R422G-C430N-L432F-L434W-S436N-dG462 | ++++ |
| I283T-P284D-N287P-S322T-P368S-N388M-E421V-C430N-L432F-L434W-S436N-dG462 | ++++ |
| I283T-P284D-N287P-S322T-N388M-R422L-C430N-L432F-L434W-S436N-dG462 | ++++ |
| I283T-P284D-N287P-S322T-N388M-C430N-L432F-L434W-S436N-dG462-A463I | ++++ |
| I283T-P284D-N287P-S322T-N388M-C430N-L432F-L434W-S436N-dG462-A463T | ++++ |
| I283T-P284D-N287P-S322T-N388M-C430N-L432F-L434W-S436N-dG462-A463F | ++++ |
| I283T-P284D-N287P-S322T-N388M-C430N-L432F-L434W-S436N-H461V-dG462 | ++++ |
| I283T-P284D-N287P-S322T-N388M-C430N-L432F-L434W-S436N-dG462-S465V | ++++ |
| I283T-P284D-N287P-S322T-N388M-C430N-L432F-L434W-S436N-H461A-dG462 | ++++ |
| I283T-P284D-N287P-S322T-N388M-C430N-L432F-L434W-S436N-H461T-dG462 | ++++ |
| I283T-P284D-N287P-S322T-N388M-C430N-L432F-L434W-S436N-H461Y-dG462 | ++++ |
| I283T-P284D-N287P-S322T-N388M-C430N-L432F-L434W-S436N-dG462-A463S | ++++ |
| I283T-P284D-N287P-S322T-N388M-C430N-L432F-L434W-S436N-H461A-dG462-A463T | ++++ |
| I283T-P284D-N287P-S322T-N388M-C430N-L432F-L434W-S436N-H461A-dG462-S465V | ++++ |
| I283T-P284D-N287P-S322T-N388M-C430N-L432F-L434W-S436N-H461A-dG462-S465I | ++++ |
| I283T-P284D-N287P-S322T-N388M-C430N-L432F-L434W-S436N-H461Y-dG462-A463T | ++++ |
| I283T-P284D-N287P-S322T-N388M-C430N-L432F-L434W-S436N-H461Y-dG462-S465V | ++++ |
| I283T-P284D-N287P-S322T-N388M-C430N-L432F-L434W-S436N-H461V-dG462-A463T | ++++ |
| I283T-P284D-N287P-S322T-N388M-C430N-L432F-L434W-S436N-H461V-dG462-S465V | ++++ |
| I283T-P284D-N287P-S322T-N388M-C430N-L432F-L434W-S436N-dG462-A463T-S465V | ++++ |
| I283T-P284D-N287P-S322T-N388M-C430N-L432F-L434W-S436N-H461A-dG462-A463T-S465V | ++++ |
| I283T-P284D-N287P-S322T-N388M-C430N-L432F-L434W-S436N-H461Y-dG462-A463T-S465V | ++++ |
| I283T-P284D-N287P-S322T-N388M-C430N-L432F-L434W-S436N-H461V-dG462-A463T-S465V | ++++ |
| I283T-P284D-N287P-S322T-N388M-E421V-R422F-C430N-L432F-L434W-S436N-dG462 | ++++ |
| I283T-P284D-N287P-S322T-N388M-E421V-C430N-L432F-L434W-S436N-H461T-dG462 | ++++ |
| I283T-P284D-N287P-S322T-N388M-E421V-C430N-L432F-L434W-S436N-H461V-dG462 | ++++ |
| I283T-P284D-N287P-S322T-N388M-E421V-C430N-L432F-L434W-S436N-dG462-A463T | ++++ |
| I283T-P284D-N287P-S322T-N388M-R422F-C430N-L432F-L434W-S436N-H461T-dG462 | ++++ |
| I283T-P284D-N287P-S322T-N388M-R422F-C430N-L432F-L434W-S436N-H461V-dG462 | ++++ |
| I283T-P284D-N287P-S322T-N388M-R422F-C430N-L432F-L434W-S436N-dG462-A463T | ++++ |
| I283T-P284D-N287P-S322T-N388M-C430N-L432F-L434W-S436N-H461T-dG462-A463T | ++++ |
| I283T-P284D-N287P-S322T-N388M-E421V-R422F-C430N-L432F-L434W-S436N-H461T-dG462 | ++++ |
| I283T-P284D-N287P-S322T-N388M-E421V-R422F-C430N-L432F-L434W-S436N-H461V-dG462 | ++++ |
| I283T-P284D-N287P-S322T-N388M-E421V-R422F-C430N-L432F-L434W-S436N-dG462-A463T | ++++ |
| I283T-P284D-N287P-S322T-N388M-E421V-C430N-L432F-L434W-S436N-H461T-dG462-A463T | ++++ |
| I283T-P284D-N287P-S322T-N388M-E421V-C430N-L432F-L434W-S436N-H461V-dG462-A463T | ++++ |
| I283T-P284D-N287P-S322T-N388M-R422F-C430N-L432F-L434W-S436N-H461T-dG462-A463T | ++++ |
| I283T-P284D-N287P-S322T-N388M-R422F-C430N-L432F-L434W-S436N-H461V-dG462-A463T | ++++ |
| I283T-P284D-N287P-S322T-N388M-E421V-R422F-C430N-L432F-L434W-S436N-H461T-dG462-A463T | ++++ |
| I283T-P284D-N287P-S322T-N388M-E421V-R422F-C430N-L432F-L434W-S436N-H461V-dG462-A463T | ++++ |
| I283T-P284D-N287P-S322T-N388M-E421V-C430N-L432F-L434W-S436N-dG462 | ++++ |
| I283T-P284D-N287P-S322T-N388M-R422F-C430N-L432F-L434W-S436N-dG462 | ++++ |
| I283T-N285P-N287T-Q290E-S322T-N388M-K394R-C430A-L432F-L434W-S436N-dH461 | ++++ |
| I283T-N285P-N287T-Q290E-S322T-N388M-K394R-C430A-L432F-L434W-S436N-dG462 | ++++ |
| I283T-N285P-N287T-Q290E-S322T-N388M-K394R-C430A-L432F-L434W-S436N-dA463 | ++++ |
| I283T-P284D-i284aP-N287P-S322T-N388M-C430N-L432F-L434W-S436N-dG462 | ++++ |
| I283T-P284D-N285M-N287T-S322T-N388M-K394R-C430A-L432F-L434W-S436N-C437M-G462P-A463P | ++++ |
| I283T-P284D-N285M-N287T-S322T-N388M-K394R-C430A-L432F-L434W-S436N-G462P-A463P-I464P | ++++ |
| I283T-P284D-N285M-N287T-L289H-S322T-N388M-K394R-C430A-L432F-L434W-S436N-G462P-A463P | ++++ |
| I283T-P284D-N285I-N287T-i287aS-S322T-N388M-K394R-C430A-L432F-L434W-S436N-G462P-A463P | ++++ |
| I283T-P284D-N285M-N287T-S322T-N388M-K394R-C430A-L432F-L434W-S436N-G462T-A463P | ++++ |
| I283T-N285P-N287T-Q290E-S322T-N388M-K394R-dR422-C430A-L432F-L434W-S436N-dG462 | ++++ |
| L223Y-I283T-N285P-N287T-Q290E-S322T-N388M-K394R-K423G-C430A-L432F-L434W-S436N-dG462 | ++++ |
| I283T-P284D-i284aP-N287P-S322T-N388M-E421V-C430N-L432F-L434W-S436N-dG462-A463T | ++++ |
| I283T-P284D-i284aP-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-H461V-dG462-A463T | ++++ |
| I283T-P284D-i284aP-N287P-S322T-N388M-E421V-R422F-C430N-L432F-L434W-S436N-dG462-A463T | ++++ |
| I283T-P284D-i284aP-N287P-S322T-N388M-E421V-R422F-C430N-L432F-L434W-S436N-H461T-dG462-A463T | ++++ |
| I283T-P284D-i284aP-N287P-S322T-N388M-E421V-R422F-C430N-L432F-L434W-S436N-H461T-dG462 | ++++ |
| I283T-P284D-i284aP-N287P-S322T-N388M-C430N-L432F-L434W-S436N-H461A-dG462-A463T | ++++ |
| I283T-P284D-i284aP-N287P-S322T-N388M-C430N-L432F-L434W-S436N-H461A-dG462 | ++++ |
| E126K-H141Q-I283T-N285P-N287T-Q290E-S322T-N388M-K394R-C430A-L432F-L434W-S436N-dG462 | ++++ |
| P184S-I283T-N285P-N287T-Q290E-S322T-N388M-K394R-C430A-L432F-L434W-S436N-dG462 | ++++ |
| L223Y-I283T-N285P-N287T-Q290E-S322T-N388M-K394R-C430A-L432F-L434W-S436N-dG462 | ++++ |
| V227Y-I283T-N285P-N287T-Q290E-S322T-N388M-K394R-C430A-L432F-L434W-S436N-dG462 | ++++ |
| I283L-N285P-N287T-Q290E-S322T-N388M-K394R-C430A-L432F-L434W-S436N-dG462 | ++++ |
| I283T-N285P-N287T-L289W-Q290E-S322T-N388M-K394R-C430A-L432F-L434W-S436N-dG462 | ++++ |
| I283T-N285P-N287T-L289R-Q290E-S322T-N388M-K394R-C430A-L432F-L434W-S436N-dG462 | ++++ |
| I283T-N285P-N287T-L289C-Q290E-S322T-N388M-K394R-C430A-L432F-L434W-S436N-dG462 | ++++ |
| I283T-N285P-N287T-Q290E-S322T-Q365T-N388M-K394R-C430A-L432F-L434W-S436N-dG462 | ++++ |
| I283T-N285P-N287T-Q290E-S322T-N388M-K394R-K423G-C430A-L432F-L434W-S436N-dG462 | ++++ |
| I283T-N285P-N287T-Q290E-S322T-N388M-K394R-C430V-L432F-L434W-S436N-dG462 | ++++ |
| I283T-N285P-N287T-Q290E-S322T-N388M-K394R-C430A-L432F-L434W-S436N-H444T-dG462 | ++++ |
| I283T-N285P-N287T-Q290E-S322T-N388M-K394R-C430A-L432F-L434W-S436N-H444R-dG462 | ++++ |
| I283T-N285P-N287T-Q290E-S322T-N388M-K394R-C430A-L432F-L434W-S436N-H444F-dG462 | ++++ |
| I283T-N285P-N287T-Q290E-S322T-N388M-K394R-C430A-L432F-L434W-S436N-N460G-dG462 | ++++ |
| I283T-N285P-N287T-Q290E-S322T-N388M-K394R-C430A-L432F-L434W-S436N-dG462-A463F | ++++ |
| I283T-N285P-N287T-Q290E-S322T-N388M-K394R-C430A-L432F-L434W-S436N-dG462-A470S | ++++ |
| I283T-N285P-N287T-Q290E-S322T-N388M-K394R-R422D-C430A-L432F-L434W-S436N-dG462 | ++++ |
| I283T-N285P-N287T-Q290E-S322T-N388M-K394R-C430A-L432F-L434W-S436N-dG462-A463V | ++++ |
| I283T-N285P-N287T-Q290E-S322T-N388M-K394R-C430A-L432F-L434W-S436N-dG462-Y471F | ++++ |
| I35V-Q140L-I283T-N285P-N287T-Q290E-S322T-N388M-K394R-C430A-L432F-L434W-S436N-dG462 | ++++ |
| E224P-I283T-N285P-N287T-Q290E-S322T-N388M-K394R-C430A-L432F-L434W-S436N-dG462 | ++++ |
| E224Q-I283T-N285P-N287T-Q290E-S322T-N388M-K394R-C430A-L432F-L434W-S436N-dG462 | ++++ |
| L222V-I283T-N285P-N287T-Q290E-S322T-N388M-K394R-C430A-L432F-L434W-S436N-dG462 | ++++ |
| V227L-I283T-N285P-N287T-Q290E-S322T-N388M-K394R-C430A-L432F-L434W-S436N-dG462 | ++++ |
| I283T-N285P-E286A-N287T-Q290E-S322T-N388M-K394R-C430A-L432F-L434W-S436N-dG462 | ++++ |
| I283T-N285P-N287T-Q290E-S322T-R361K-N388M-K394R-C430A-L432F-L434W-S436N-dG462 | ++++ |
| I283T-N285P-N287T-Q290E-S322T-M355V-N388M-K394R-C430A-L432F-L434W-S436N-dG462 | ++++ |
| I283T-N285P-N287T-Q290E-S322T-M355T-N388M-K394R-C430A-L432F-L434W-S436N-dG462 | ++++ |
| I283T-N285P-N287T-Q290E-S322T-M355C-N388M-K394R-C430A-L432F-L434W-S436N-dG462 | ++++ |
| I283T-N285P-N287T-Q290E-S322T-N388M-K394R-E421W-C430A-L432F-L434W-S436N-dG462 | ++++ |
| I283T-N285P-N287T-Q290E-S322T-N388S-K394R-C430A-L432F-L434W-S436N-dG462 | ++++ |
| I283T-N285P-N287T-Q290E-S322T-N388M-K394H-C430A-L432F-L434W-S436N-dG462 | ++++ |
| I283T-N285P-N287T-Q290E-S322T-N388M-K394R-H419P-C430A-L432F-L434W-S436N-dG462 | ++++ |
| I283T-N285P-N287T-Q290E-S322T-N388M-K394R-H419A-C430A-L432F-L434W-S436N-dG462 | ++++ |
| I283T-N285P-N287T-Q290E-S322T-N388M-K394R-E421L-C430A-L432F-L434W-S436N-dG462 | ++++ |
| I283T-N285P-N287T-Q290E-S322T-N388M-K394R-S429V-C430A-L432F-L434W-S436N-dG462 | ++++ |
| I283T-N285P-N287T-Q290E-S322T-N388M-K394R-C430A-I431K-L432F-L434W-S436N-dG462 | ++++ |
| I283T-N285P-N287T-Q290E-S322T-N388M-K394R-C430A-I431N-L432F-L434W-S436N-dG462 | ++++ |
| I283T-N285P-N287T-Q290E-S322T-N388M-K394R-C430A-I431P-L432F-L434W-S436N-dG462 | ++++ |
| I283T-N285P-N287T-Q290E-S322T-N388M-K394R-C430A-I431C-L432F-L434W-S436N-dG462 | ++++ |
| I283T-N285P-N287T-Q290E-S322T-N388M-K394R-C430A-I431T-L432F-L434W-S436N-dG462 | ++++ |
| I283T-N285P-N287T-Q290E-S322T-N388M-K394R-C430A-I431A-L432F-L434W-S436N-dG462 | ++++ |
| I283T-N285P-N287T-Q290E-S322T-N388M-K394R-C430S-L432F-L434W-S436N-dG462 | ++++ |
| I283T-N285P-N287T-Q290E-S322T-N388M-K394R-C430A-L432F-L434W-S436N-C437L-dG462 | ++++ |
| I283T-N285P-N287T-Q290E-S322T-N388M-K394R-C430A-L432F-L434W-S436N-Y438W-dG462 | ++++ |
| I283T-N285P-N287T-Q290E-S322T-N388M-K394R-C430A-L432F-L434W-S436N-C437T-dG462 | ++++ |
| I283T-N285P-N287T-Q290E-S322T-N388M-K394R-C430A-L432F-L434W-S436N-C437M-dG462 | ++++ |
| I283T-N285P-N287T-Q290E-S322T-N388M-K394R-C430A-L432F-L434W-S436N-C437I-dG462 | ++++ |
| I283T-N285P-N287T-Q290E-S322T-N388M-K394R-C430A-L432F-L434W-S436N-Y438M-dG462 | ++++ |
| I283T-N285P-N287T-Q290E-S322T-N388M-K394R-C430A-L432F-L434W-S436N-S439P-dG462 | ++++ |
| I283T-N285P-N287T-Q290E-S322T-N388M-K394R-C430A-L432F-L434W-S436N-K447T-dG462 | ++++ |
| D27N-I283T-N285P-N287T-Q290E-S322T-N388M-K394R-C430A-L432F-L434W-S436N-G448T-dG462 | ++++ |
| I283T-N285P-N287T-Q290E-S322T-N388M-K394R-C430A-L432F-L434W-S436N-G448C-dG462 | ++++ |
| I283T-N285P-N287T-Q290E-S322T-N388M-K394R-E421F-C430A-L432F-L434W-S436N-dG462 | ++++ |
| I283T-N285P-N287T-Q290E-S322T-N388M-K394R-i421aD-C430A-L432F-L434W-S436N-dG462 | ++++ |
| I283T-N285P-N287T-Q290E-S322T-N388M-K394R-i421aT-C430A-L432F-L434W-S436N-dG462 | ++++ |
| I283T-N285P-N287T-Q290E-S322T-N388M-K394R-i421aS-C430A-L432F-L434W-S436N-dG462 | ++++ |
| I283T-N285P-N287T-Q290E-S322T-N388M-K394R-i421aI-C430A-L432F-L434W-S436N-dG462 | ++++ |
| I283T-N285P-N287T-Q290E-S322T-N388M-K394R-i421aE-C430A-L432F-L434W-S436N-dG462 | ++++ |
| I283T-N285P-N287T-Q290E-S322T-N388M-K394R-i421aR-C430A-L432F-L434W-S436N-dG462 | ++++ |
| I283T-N285P-N287T-Q290E-S322T-N388M-K394R-i421aP-C430A-L432F-L434W-S436N-dG462 | ++++ |
| I283T-N285P-N287T-Q290E-S322T-N388M-K394R-i424aA-C430A-L432F-L434W-S436N-dG462 | ++++ |
| I283T-N285P-N287T-Q290E-S322T-N388M-K394R-i424aW-C430A-L432F-L434W-S436N-dG462 | ++++ |
| I283T-N285P-N287T-Q290E-S322T-N388M-K394R-i424aL-C430A-L432F-L434W-S436N-dG462 | ++++ |
| I283T-N285P-N287T-Q290E-S322T-N388M-K394R-i424aS-C430A-L432F-L434W-S436N-dG462 | ++++ |
| I283T-N285P-N287T-Q290E-S322T-N388M-K394R-i424aD-C430A-L432F-L434W-S436N-dG462 | ++++ |
| I283T-N285P-N287T-Q290E-S322T-N388M-K394R-i426aL-C430A-L432F-L434W-S436N-dG462 | ++++ |
| I283T-N285P-N287T-Q290E-S322T-N388M-K394R-i429aN-C430A-L432F-L434W-S436N-dG462 | ++++ |
| I283T-N285P-N287T-Q290E-S322T-N388M-K394R-C430A-i430aC-L432F-L434W-S436N-dG462 | ++++ |
| I283T-N285P-N287T-Q290E-S322T-N388M-K394R-C430A-i430aG-L432F-L434W-S436N-dG462 | ++++ |
| I283T-N285P-N287T-Q290E-S322T-N388M-K394R-C430A-i430aN-L432F-L434W-S436N-dG462 | ++++ |
| I283T-N285P-N287T-Q290E-S322T-N388M-K394R-C430A-i430aS-L432F-L434W-S436N-dG462 | ++++ |
| I283T-N285P-N287T-Q290E-S322T-N388M-K394R-C430A-i430aR-L432F-L434W-S436N-dG462 | ++++ |
| I283T-N285P-N287T-Q290E-S322T-N388M-K394R-C430A-i430aL-L432F-L434W-S436N-dG462 | ++++ |
| I283T-N285P-N287T-Q290E-S322T-N388M-K394R-C430A-i430aI-L432F-L434W-S436N-dG462 | ++++ |
| I283T-N285P-N287T-Q290E-S322T-N388M-K394R-C430A-i430aQ-L432F-L434W-S436N-dG462 | ++++ |
| I283T-N285P-N287T-Q290E-S322T-N388M-K394R-C430A-i430aH-L432F-L434W-S436N-dG462 | ++++ |
| I283T-N285P-N287T-Q290E-S322T-N388M-K394R-C430A-L432F-L434W-S436N-i438aD-dG462 | ++++ |
| I283T-N285P-N287T-Q290E-S322T-N388M-K394R-C430A-L432F-L434W-S436N-i443aR-dG462 | ++++ |
| I283T-N285P-N287T-Q290E-S322T-N388M-K394R-C430A-L432F-L434W-S436N-i443aV-dG462 | ++++ |
| I283T-N285P-N287T-Q290E-S322T-N388M-K394R-C430A-L432F-L434W-S436N-i443aL-dG462 | ++++ |
| I283T-N285P-N287T-Q290E-S322T-N388M-K394R-C430A-L432F-L434W-S436N-i443aT-dG462 | ++++ |
| I283T-N285P-N287T-Q290E-S322T-N388M-K394R-C430A-L432F-L434W-S436N-i444aH-dG462 | ++++ |
| I283T-N285P-N287T-Q290E-S322T-N388M-K394R-C430A-L432F-L434W-S436N-i444aP-dG462 | ++++ |
| I283T-N285P-N287T-Q290E-S322T-N388M-K394R-C430A-L432F-L434W-S436N-i444aV-dG462 | ++++ |
| I283T-N285P-N287T-Q290E-S322T-N388M-K394R-C430A-L432F-L434W-S436N-i444aR-dG462 | ++++ |
| I283T-N285P-N287T-Q290E-S322T-N388M-K394R-C430A-L432F-L434W-S436N-i444aN-dG462 | ++++ |
| I283T-N285P-N287T-Q290E-S322T-N388M-K394R-C430A-L432F-L434W-S436N-i444aG-dG462 | ++++ |
| I283T-N285P-N287T-Q290E-S322T-N388M-K394R-C430A-L432F-L434W-S436N-i444aT-dG462 | ++++ |
| I283T-N285P-N287T-Q290E-S322T-N388M-K394R-C430A-L432F-L434W-S436N-i444aL-dG462 | ++++ |
| I283T-N285P-N287T-Q290E-S322T-N388M-K394R-C430A-L432F-L434W-S436N-i444aE-dG462 | ++++ |
| I283T-N285P-N287T-Q290E-S322T-N388M-K394R-C430A-L432F-L434W-S436N-i444aK-dG462 | ++++ |
| I283T-N285P-N287T-Q290E-S322T-N388M-K394R-C430A-L432F-L434W-S436N-i444aS-dG462 | ++++ |
| I283T-N285P-N287T-Q290E-S322T-N388M-K394R-C430A-L432F-L434W-S436N-i445aQ-dG462 | ++++ |
| I283T-N285P-N287T-Q290E-S322T-N388M-K394R-C430A-L432F-L434W-S436N-i447aY-dG462 | ++++ |
| I283T-N285P-N287T-Q290E-S322T-N388M-K394R-C430A-L432F-L434W-S436N-i447aL-dG462 | ++++ |
| I283T-N285P-N287T-Q290E-S322T-N388M-K394R-C430A-L432F-L434W-S436N-i447aK-dG462 | ++++ |
| I283T-N285P-N287T-Q290E-S322T-N388M-K394R-C430A-L432F-L434W-S436N-i447aP-dG462 | ++++ |
| I283T-N285P-N287T-Q290E-S322T-N388M-K394R-C430A-L432F-L434W-S436N-i448aS-dG462 | ++++ |
| I283T-N285P-N287T-Q290E-S322T-N388M-K394R-C430A-i431aG-L432F-L434W-S436N-dG462 | ++++ |
| I283T-N285P-N287T-Q290E-S322T-N388M-K394R-C430A-i431aL-L432F-L434W-S436N-dG462 | ++++ |
| I283T-N285P-N287T-Q290E-S322T-N388M-K394R-C430A-i431aN-L432F-L434W-S436N-dG462 | ++++ |
| I283T-N285P-N287T-Q290E-S322T-N388M-K394R-C430A-L432F-L434W-S436N-i449aS-dG462 | ++++ |
| L223Y-I283T-N285P-N287T-Q290E-S322T-N388M-K394R-E421F-C430A-L432F-L434W-S436N-dG462 | ++++ |
| I283T-N285P-N287T-Q290E-S322T-N388M-K394R-E421F-C430A-i430aG-L432F-L434W-S436N-dG462 | ++++ |
| I283T-N285P-N287T-Q290E-S322T-N388M-K394R-E421F-C430A-i430aL-L432F-L434W-S436N-dG462 | ++++ |
| I283T-N285P-N287T-Q290E-S322T-N388M-K394R-E421F-C430A-i430aN-L432F-L434W-S436N-dG462 | ++++ |
| I283T-N285P-N287T-Q290E-S322T-N388M-K394R-E421F-C430A-L432F-L434W-S436N-i448aS-dG462 | ++++ |
| L223Y-I283T-N285P-N287T-Q290E-S322T-N388M-K394R-E421F-K423G-C430A-L432F-L434W-S436N-dG462 | ++++ |
| L223Y-I283T-N285P-N287T-Q290E-S322T-N388M-K394R-K423G-C430A-i430aG-L432F-L434W-S436N-dG462 | ++++ |
| L223Y-I283T-N285P-N287T-Q290E-S322T-N388M-K394R-K423G-C430A-i430aL-L432F-L434W-S436N-dG462 | ++++ |
| L223Y-I283T-N285P-N287T-Q290E-S322T-N388M-K394R-K423G-C430A-i430aN-L432F-L434W-S436N-dG462 | ++++ |
| L223Y-I283T-N285P-N287T-Q290E-S322T-N388M-K394R-K423G-C430A-L432F-L434W-S436N-i448aS-dG462 | ++++ |
| L223Y-I283T-N285P-N287T-Q290E-S322T-N388M-K394R-i429aN-C430A-L432F-L434W-S436N-dG462 | ++++ |
| L223Y-I283T-N285P-N287T-Q290E-S322T-N388M-K394R-C430A-i430aG-L432F-L434W-S436N-dG462 | ++++ |
| L223Y-I283T-N285P-N287T-Q290E-S322T-N388M-K394R-i429aN-i429bG-C430A-L432F-L434W-S436N-dG462 | ++++ |
| I283T-N285P-N287T-Q290E-S322T-N388M-K394R-C430A-i430aG-L432F-L434W-S436N-C437L-dG462 | ++++ |
| L223Y-I283T-N285P-N287T-Q290E-S322T-N388M-K394R-C430A-i430aG-L432F-L434W-S436N-C437L-dG462 | ++++ |
| L223Y-I283T-N285P-N287T-Q290E-S322T-N388M-K394R-C430A-i430aN-L432F-L434W-S436N-dG462 | ++++ |
| L223Y-I283T-N285P-N287T-Q290E-S322T-N388M-K394R-C430A-L432F-L434W-S436N-C437L-dG462 | ++++ |
| L223Y-I283T-N285P-N287T-Q290E-S322T-N388M-K394R-C430A-i430aL-L432F-L434W-S436N-dG462 | ++++ |
| I283T-N285P-N287T-Q290E-S322T-N388M-K394R-C430A-i430aG-L432F-L434W-S436N-V451S-dG462 | ++++ |
| I283T-N285P-N287T-Q290E-S322T-N388M-K394R-i425aW-C430A-i430aG-L432F-L434W-S436N-dG462 | ++++ |
| H141C-I283T-N285P-N287T-Q290E-S322T-N388M-K394R-C430A-i430aG-L432F-L434W-S436N-dG462 | ++++ |
| L222V-I283T-N285P-N287T-Q290E-S322T-N388M-K394R-C430A-i430aG-L432F-L434W-S436N-dG462 | ++++ |
| I283T-N285P-N287T-Q290E-S322T-I330V-N388M-K394R-C430A-i430aG-L432F-L434W-S436N-dG462 | ++++ |
| I283T-N285P-N287T-Q290E-S322T-N388M-S389H-K394R-C430A-i430aG-L432F-L434W-S436N-dG462 | ++++ |
| H141C-I283T-N285P-N287T-Q290E-S322T-N388M-S389H-K394R-C430A-i430aG-L432F-L434W-S436N-dG462 | ++++ |
| I283T-N285P-N287T-Q290E-S322T-I330V-N388M-S389H-K394R-C430A-i430aG-L432F-L434W-S436N-dG462 | ++++ |
| H141C-I283T-N285P-N287T-Q290E-S322T-I330V-N388M-S389H-K394R-C430A-i430aG-L432F-L434W-S436N-dG462 | ++++ |
| I283T-N285P-N287T-Q290E-S322T-I330V-N388M-S389H-K394R-C430A-i430aG-L432F-L434W-S436N-V451S-dG462 | ++++ |
| E224P-I283T-N285P-N287T-Q290E-S322T-N388M-K394R-C430A-i430aG-L432F-L434W-S436N-dG462 | ++++ |
| L222V-E224P-I283T-N285P-N287T-Q290E-S322T-N388M-K394R-C430A-i430aG-L432F-L434W-S436N-dG462 | ++++ |
| I283T-N285P-N287T-Q290E-S322T-N388M-S389H-K394R-C430A-i430aG-L432F-L434W-S436N-I453L-dG462 | ++++ |
| E224A-I283T-N285P-N287T-Q290E-S322T-N388M-S389H-K394R-C430A-i430aG-L432F-L434W-S436N-dG462 | ++++ |
| E224P-I283T-N285P-N287T-Q290E-S322T-N388M-S389H-K394R-C430A-i430aG-L432F-L434W-S436N-dG462 | ++++ |
| I283T-P284D-i284aP-N287P-S322T-N388M-E421V-i421aR-C430N-L432F-L434W-S436N-H461T-dG462-A463T | ++++ |
| I283T-P284D-i284aP-N287P-S322T-N388M-E421V-i422aK-C430N-L432F-L434W-S436N-H461T-dG462-A463T | ++++ |
| I283T-P284D-i284aP-N287P-S322T-N388M-E421V-C430N-I431V-L432F-L434W-S436N-H461T-dG462-A463T | ++++ |
| I283T-P284D-i284aP-N287P-S322T-Q365I-N388M-E421V-C430N-L432F-L434W-S436N-H461T-dG462-A463T | ++++ |
| I283T-P284D-i284aP-N287P-S322T-Q385G-N388M-E421V-C430N-L432F-L434W-S436N-H461T-dG462-A463T | ++++ |
| I283T-P284D-i284aP-N287P-S322T-Q385L-N388M-E421V-C430N-L432F-L434W-S436N-H461T-dG462-A463T | ++++ |
| I283T-P284D-i284aP-N287P-S322T-Q385S-N388M-E421V-C430N-L432F-L434W-S436N-H461T-dG462-A463T | ++++ |
| I283T-P284D-i284aP-N287P-S322T-Q385T-N388M-E421V-C430N-L432F-L434W-S436N-H461T-dG462-A463T | ++++ |
| I283T-P284D-i284aP-N287P-S322T-H384G-N388M-E421V-C430N-L432F-L434W-S436N-H461T-dG462-A463T | ++++ |
| I283T-P284D-i284aP-N287P-S322T-H384L-N388M-E421V-C430N-L432F-L434W-S436N-H461T-dG462-A463T | ++++ |
| I283T-P284D-i284aP-N287P-L289R-S322T-N388M-E421V-C430N-L432F-L434W-S436N-H461T-dG462-A463T | ++++ |
| I283T-P284D-i284aP-E286A-N287P-S322T-N388M-E421V-C430N-L432F-L434W-S436N-H461T-dG462-A463T | ++++ |
| I283T-P284D-i284aP-E286S-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-H461T-dG462-A463T-D468L | ++++ |
| I283T-P284D-i284aP-N287P-S322T-N388M-E421V-C430N-L432F-L434W-S436N-G448C-H461T-dG462-A463T | ++++ |
| I283T-P284D-i284aP-N287P-S322T-N388M-E421V-C430N-L432F-L434W-S436N-G448E-H461T-dG462-A463T | ++++ |
| I283T-P284D-i284aP-N287P-S322T-N388M-E421V-C430N-L432F-L434W-S436N-G448V-H461T-dG462-A463T | ++++ |
| I283T-P284D-i284aP-N287P-S322T-N388M-E421V-C430N-L432F-L434W-S436N-H461T-dG462-A463T-Y469A | ++++ |
| I283T-P284D-i284aP-N287P-S322T-N388M-E421V-C430N-L432F-L434W-S436N-H461T-dG462-A463T-Y469H | ++++ |
| I283T-P284D-i284aP-N287P-S322T-N388M-E421V-C430N-L432F-L434W-S436N-C437F-H461T-dG462-A463T | ++++ |
| I283T-P284D-i284aP-N287P-S322T-N388M-E421V-C430N-L432F-L434W-S436N-C437G-H461T-dG462-A463T | ++++ |
| I283T-P284D-i284aP-N287P-S322T-N388M-E421V-C430N-i430aR-L432F-L434W-S436N-H461T-dG462-A463T | ++++ |
| I283T-P284D-i284aP-N287P-S322T-N388M-E421V-C430N-I431M-L432F-L434W-S436N-H461T-dG462-A463T | ++++ |
| P45A-I283T-P284D-i284aP-N287P-S322T-N388M-E421V-C430N-L432F-L434W-S436N-i443aG-i443bA-H461T-dG462-A463T | ++++ |
| I283T-P284D-i284aP-N287P-S322T-N388M-E421V-C430N-L432F-L434W-S436N-H461T-G462R-i462aA-A463T | ++++ |
| R15L-I283T-P284D-i284aP-N287P-S322T-K378M-N388M-E421V-C430N-L432F-L434W-S436N-H461T-dG462-A463T | ++++ |
| V272L-I283T-P284D-i284aP-N287P-S322T-N388M-E421V-C430N-L432F-L434W-S436N-H461T-dG462-A463T | ++++ |
| V272R-I283T-P284D-i284aP-N287P-S322T-N388M-E421V-C430N-L432F-L434W-S436N-H461T-dG462-A463T | ++++ |
| V272I-I283T-P284D-i284aP-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-C437F-G448E-H461T-dG462-A463T | ++++ |
| I283T-P284D-i284aP-N287P-S322T-N388M-E421V-C430N-L432F-L434W-S436N-C437F-G448E-H461T-G462R-i462aA-A463T | ++++ |
| I283T-P284D-i284aP-N287P-S322T-N388M-E421V-C430N-L432F-L434W-S436N-C437F-H461T-G462R-i462aA-A463T | ++++ |
| I283T-P284D-i284aP-N287P-S322T-N388M-E421V-C430N-L432F-L434W-S436N-G448E-H461T-G462R-i462aA-A463T | ++++ |
| V272R-I283T-P284D-i284aP-N287P-S322T-N388M-E421V-C430N-L432F-L434W-S436N-C437F-H461T-dG462-A463T | ++++ |
| V272R-I283T-P284D-i284aP-N287P-S322T-N388M-E421V-C430N-L432F-L434W-S436N-C437F-G448E-H461T-dG462-A463T | ++++ |
| V272R-I283T-P284D-i284aP-N287P-S322T-N388M-E421V-C430N-L432F-L434W-S436N-C437F-G448E-H461T-G462R-i462aA-A463T | ++++ |
| V272R-I283T-P284D-i284aP-N287P-S322T-N388M-E421V-C430N-L432F-L434W-S436N-C437F-H461T-G462R-i462aA-A463T | ++++ |
| V272R-I283T-P284D-i284aP-N287P-S322T-N388M-E421V-C430N-L432F-L434W-S436N-G448E-H461T-dG462-A463T | ++++ |
| V272R-I283T-P284D-i284aP-N287P-S322T-N388M-E421V-C430N-L432F-L434W-S436N-G448E-H461T-G462R-i462aA-A463T | ++++ |
| V272R-I283T-P284D-i284aP-N287P-S322T-N388M-E421V-C430N-L432F-L434W-S436N-H461T-G462R-i462aA-A463T | ++++ |
| V272I-I283T-P284D-i284aP-N287P-S322T-N388M-E421V-C430N-L432F-L434W-S436N-C437F-H461T-G462R-i462aA-A463T | ++++ |
| V272K-I283T-P284D-i284aP-N287P-S322T-N388M-E421V-C430N-L432F-L434W-S436N-C437F-H461T-G462R-i462aA-A463T | ++++ |
| A33P-I283T-P284D-i284aP-N287P-S322T-N388M-E421V-C430N-L432F-L434W-S436N-C437F-H461T-G462R-i462aA-A463T | ++++ |
| I283T-P284D-i284aP-N287P-S322T-H384S-N388M-E421V-C430N-L432F-L434W-S436N-C437F-H461T-G462R-i462aA-A463T | ++++ |
| I283T-P284D-i284aP-N287P-S322T-N388M-I418V-E421V-C430N-L432F-L434W-S436N-C437F-H461T-G462R-i462aA-A463T | ++++ |
| K43E-I283T-P284D-i284aP-N287P-S322T-N388M-E421V-C430P-L432F-L434W-S436N-C437F-H461T-G462R-i462aA-A463T | ++++ |
| I283T-P284D-i284aP-N287P-S322T-N388M-E421V-C430R-L432F-L434W-S436N-C437F-H461T-G462R-i462aA-A463T | ++++ |
| I283T-P284D-i284aP-N287P-S322T-N388M-E421V-C430N-L432F-L434W-S436N-C437F-Y438L-H461T-G462R-i462aA-A463T | ++++ |
| I283T-P284D-i284aP-N287P-S322T-N388M-E421V-C430N-L432F-L434W-S436Q-C437F-H461T-G462R-i462aA-A463T | ++++ |
| I283T-P284D-i284aP-N287P-S322T-N388M-E421V-C430N-L432F-L434W-S436N-C437F-E441V-H461T-G462R-i462aA-A463T | ++++ |
| I283T-P284D-i284aP-N287P-S322T-N388M-E421V-C430N-L432F-L434W-S436N-C437F-i444aT-H461T-G462R-i462aA-A463T | ++++ |
| I283T-P284D-i284aP-N287P-S322T-N388M-E421V-C430R-L432F-L434W-S436N-C437F-Y438L-H461T-G462R-i462aA-A463T | ++++ |
| V272I-I283T-P284D-i284aP-N287P-S322T-N388M-E421V-C430R-L432F-L434W-S436N-C437F-H461T-G462R-i462aA-A463T | ++++ |
| V272I-I283T-P284D-i284aP-N287P-S322T-N388M-E421V-C430R-L432F-L434W-S436N-C437F-Y438L-H461T-G462R-i462aA-A463T | ++++ |
| V272I-I283T-P284D-i284aP-N287P-S322T-N388M-E421V-C430N-L432F-L434W-S436N-C437F-Y438L-H461T-G462R-i462aA-A463T | ++++ |
| V272R-I283T-P284D-i284aP-N287P-S322T-N388M-I418V-E421V-C430N-L432F-L434W-S436N-G448E-H461T-G462R-i462aA-A463T | ++++ |
| G17S-V272R-I283T-P284D-i284aP-N287P-S322T-N388M-E421K-C430N-L432F-L434W-S436N-G448E-H461T-G462R-i462aA-A463T | ++++ |
| V272R-I283T-P284D-i284aP-N287P-S322T-N388M-E421V-E428T-C430N-L432F-L434W-S436N-G448E-H461T-G462R-i462aA-A463T | ++++ |
| V272R-I283T-P284D-i284aP-N287P-S322T-N388M-E421V-C430N-L432F-L434W-S436N-E441H-G448E-H461T-G462R-i462aA-A463T | ++++ |
| V46A-V272R-I283T-P284D-i284aP-N287P-S322T-N388M-E421V-C430N-L432F-L434W-S436N-G448E-i458aG-H461T-G462R-i462aA- | ++++ |
| A463T | |
| S39G-V272R-I283T-P284D-i284aP-N287P-S322T-N388M-E421V-F426L-E428T-C430N-L432F-L434W-S436N-G448E-H461T-G462R- | ++++ |
| i462aA-A463T | |
| V272R-I283T-P284D-i284aP-N287P-S322T-N388M-E421V-E428T-C430N-L432F-L434W-S436N-K447I-G448E-H461T-G462R-i462aA- | |
| A463T | ++++ |
| E224P-I283T-N285P-N287T-Q290E-S322T-N388M-S389H-K394R-C430A-i430aG-L432F-L434W-S436N-I453L-dG462 | ++++ |

The list of mutations (substitutions) involved in the variants of the Yersinia frederiksenii Ferulic acid decarboxylase with increased activity is described in Table 3.

The list of deletions involved in the variants of the Yersinia frederiksenii Ferulic acid decarboxylase with increased activity is described in Table 4.

The list of insertions involved in the variants of the Yersinia frederiksenii Ferulic acid decarboxylase with increased activity is described in Table 5.

TABLE 3
List of the mutated positions involved in the variants of Yersinia
frederiksenii Ferulic acid decarboxylase with increased activity

| Position | Wild-Type amino Acid | Mutations |
|---|---|---|
| 6 | L | M |
| 7 | R | C |
| 15 | R | H, L |
| 17 | G | S |
| 25 | P | L |
| 27 | D | N |
| 33 | A | P |
| 35 | I | V |
| 39 | S | G |
| 43 | K | E |
| 45 | P | A, Q |
| 46 | V | A, I |
| 48 | L | M |
| 51 | N | D |
| 72 | E | K |
| 89 | S | N |
| 126 | E | K |
| 140 | Q | L |
| 141 | H | C, L, Q, S, Y |
| 144 | N | S |
| 183 | F | Y |
| 184 | P | A, S |
| 185 | S | T |
| 189 | S | C |
| 222 | L | V |
| 223 | L | Y |
| 224 | E | A, P, Q |
| 227 | V | L, Y |
| 272 | V | I, K, L, R |
| 283 | I | C, F, H, L, Q, S, T, V |
| 284 | P | D, E |
| 285 | N | A, H, I, L, M, P, S, T, V |
| 286 | E | A, C, D, G, H, N, S, W |
| 287 | N | I, K, P, Q, R, S, T, V |
| 288 | S | N, T |
| 289 | L | A, C, H, M, Q, R, W, Y |
| 290 | Q | E |
| 292 | I | L, V |
| 321 | C | T, V |
| 322 | S | T |
| 327 | A | S |
| 329 | A | S |
| 330 | I | V |
| 331 | Q | G |
| 332 | N | H, R, S |
| 333 | V | M |
| 336 | T | H, N, Q |
| 337 | A | T |
| 338 | N | G |
| 355 | M | C, T, V |
| 361 | R | K |
| 365 | Q | I, M, S, T |
| 366 | T | A |
| 368 | P | S |
| 378 | K | M |
| 379 | V | I |
| 380 | G | V |
| 382 | V | A, L |
| 384 | H | A, G, L, S, T |
| 385 | Q | D, F, G, L, P, R, S, T, W |
| 387 | R | A |
| 388 | N | M, Q, S |
| 389 | S | A, H, Q |
| 391 | K | I, L |
| 392 | L | Y |
| 393 | P | A |
| 394 | K | A, C, G, H, L, Q, R |
| 395 | I | V |
| 418 | I | V |
| 419 | H | A, P |
| 421 | E | F, K, L, T, V, W |
| 422 | R | D, F, G, H, L, Q, S, V |
| 423 | K | C, G |
| 426 | F | H, I, L, T, V, W, Y |
| 428 | E | M, T |
| 429 | S | A, D, V |
| 430 | C | A, K, N, P, R, S, T, V |
| 431 | I | A, C, K, M, N, P, T, V |
| 432 | L | A, F, W |
| 434 | L | F, M, V, W |
| 436 | S | A, M, N, Q, T, W |
| 437 | C | F, G, I, L, M, T, V |
| 438 | Y | L, M, W |
| 439 | S | L, P |
| 440 | E | D, I |
| 441 | E | D, G, H, I, L, P, T, V |
| 443 | R | H |
| 444 | H | F, I, L, M, N, R, T, V |
| 445 | N | A, Q, T |
| 446 | R | D, G, P |
| 447 | K | F, H, I, T, W |
| 448 | G | C, E, T, V |
| 449 | K | N, W |
| 451 | V | S |
| 453 | I | L |
| 457 | L | A |
| 458 | P | C, E, I, L, M, Q, R |
| 459 | E | G, L, P, R, T |
| 460 | N | G, H, I, L, M, V, Y |
| 461 | H | A, D, E, G, N, S, T, V, Y |
| 462 | G | A, C, I, P, R, T, V |
| 463 | A | E, F, G, I, L, M, P, S, T, V |
| 464 | I | P |
| 465 | S | A, I, V |
| 467 | F | W |
| 468 | D | L |
| 469 | Y | A, D, E, H, N, S, T |
| 470 | A | G, S, T |
| 471 | Y | F |

TABLE 4
List of the deleted positions involved in the variants of Yersinia
frederiksenii Ferulic acid decarboxylase with increased activity

| Position Deleted | Wild-Type amino Acid |
|---|---|
| 422 | R |
| 458 | P |
| 459 | E |
| 460 | N |
| 461 | H |
| 462 | G |
| 463 | A |

TABLE 5
List of the inserted positions involved in the variants of Yersinia
frederiksenii 3- Ferulic acid decarboxylase with increased activity

| Position | Insertions |
|---|---|
| 284 | P |
| 287 | S |
| 421 | D, E, G, I, P, R, S, T |
| 422 | K |
| 424 | A, D, L, S, W |
| 425 | W |
| 426 | L |
| 429 | G, N |
| 430 | C, G, H, I, L, N, Q, R, S |
| 431 | G, L, N |
| 438 | D |
| 443 | A, G, L, R, T, V |
| 444 | E, G, H, K, L, N, P, R, S, T, V |
| 445 | Q |
| 447 | K, L, P, Y |
| 448 | S |
| 449 | S |
| 458 | G |
| 462 | A |

1. A variant of a 3-methylcrotonic acid decarboxylase (MDC) showing an improved activity in converting 3-methylcrotonic acid into isobutene over the corresponding MDC from which it is derived and having an amino acid sequence as shown in SEQ ID NO:1 or an amino acid sequence having at least 55% sequence identity to SEQ ID NO:1, in which one or more amino acid residues at a position selected from the group consisting of positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470 and 471 in the amino acid sequence shown in SEQ ID NO:1 or at a position corresponding to any of these positions, are substituted with another amino acid residue or deleted or wherein an insertion has been effected at one or more of these positions.

2. The MDC variant of claim 1, wherein
(1) an amino acid residue at position 7 in the amino acid sequence shown in SEQ ID NO: 1 or at a position corresponding to this position, is deleted or substituted with cysteine; and/or
(2) an amino acid residue at position 17 in the amino acid sequence shown in SEQ ID NO: 1 or at a position corresponding to this position, is deleted or substituted with serine; and/or
(3) an amino acid residue at position 27 in the amino acid sequence shown in SEQ ID NO: 1 or at a position corresponding to this position, is deleted or substituted with asparagine; and/or
(4) an amino acid residue at position 33 in the amino acid sequence shown in SEQ ID NO: 1 or at a position corresponding to this position, is deleted or substituted with proline; and/or
(5) an amino acid residue at position 35 in the amino acid sequence shown in SEQ ID NO: 1 or at a position corresponding to this position, is deleted or substituted with valine; and/or
(6) an amino acid residue at position 45 in the amino acid sequence shown in SEQ ID NO: 1 or at a position corresponding to this position, is deleted or substituted with alanine or glutamine; and/or
(7) an amino acid residue at position 46 in the amino acid sequence shown in SEQ ID NO: 1 or at a position corresponding to this position, is deleted or substituted with alanine or isoleucine; and/or
(8) an amino acid residue at position 48 in the amino acid sequence shown in SEQ ID NO: 1 or at a position corresponding to this position, is deleted or substituted with methionine; and/or
(9) an amino acid residue at position 51 in the amino acid sequence shown in SEQ ID NO: 1 or at a position corresponding to this position, is deleted or substituted with aspartate; and/or
(10) an amino acid residue at position 140 in the amino acid sequence shown in SEQ ID NO: 1 or at a position corresponding to this position, is deleted or substituted with leucine; and/or
(11) an amino acid residue at position 144 in the amino acid sequence shown in SEQ ID NO: 1 or at a position corresponding to this position, is deleted or substituted with serine; and/or
(12) an amino acid residue at position 183 in the amino acid sequence shown in SEQ ID NO: 1 or at a position corresponding to this position, is deleted or substituted with tyrosine; and/or
(13) an amino acid residue at position 184 in the amino acid sequence shown in SEQ ID NO: 1 or at a position corresponding to this position, is deleted or substituted with alanine or serine; and/or
(14) an amino acid residue at position 185 in the amino acid sequence shown in SEQ ID NO: 1 or at a position corresponding to this position, is deleted or substituted with threonine; and/or
(15) an amino acid residue at position 122 in the amino acid sequence shown in SEQ ID NO: 1 or at a position corresponding to this position, is deleted or substituted with valine; and/or
(16) an amino acid residue at position 227 in the amino acid sequence shown in SEQ ID NO: 1 or at a position corresponding to this position, is deleted or substituted with leucine or tyrosine; and/or
(17) an amino acid residue at position 284 in the amino acid sequence shown in SEQ ID NO: 1 or at a position corresponding to this position, is deleted or substituted with aspartate or glutamate or a proline is inserted; and/or
(18) an amino acid residue at position 285 in the amino acid sequence shown in SEQ ID NO: 1 or at a position corresponding to this position, is deleted or substituted with alanine, histidine, isoleucine, leucine, methionine, proline, serine, threonine or valine; and/or
(19) an amino acid residue at position 286 in the amino acid sequence shown in SEQ ID NO: 1 or at a position corresponding to this position, is deleted or substituted with alanine, cysteine, aspartate, glycine, histidine, asparagine, serine or tryptophan; and/or
(20) an amino acid residue at position 287 in the amino acid sequence shown in SEQ ID NO: 1 or at a position corresponding to this position, is deleted or substituted with isoleucine, lysine, proline, glutamine, arginine, serine, threonine or valine or a serine is inserted; and/or
(21) an amino acid residue at position 288 in the amino acid sequence shown in SEQ ID NO: 1 or at a position corresponding to this position, is deleted or substituted with asparagine or threonine; and/or
(22) an amino acid residue at position 289 in the amino acid sequence shown in SEQ ID NO: 1 or at a position corresponding to this position, is deleted or substituted with alanine, cysteine, histidine, methionine, glutamine, arginine, tryptophan or tyrosine; and/or
(23) an amino acid residue at position 290 in the amino acid sequence shown in SEQ ID NO: 1 or at a position corresponding to this position, is deleted or substituted with glutamate; and/or
(24) an amino acid residue at position 292 in the amino acid sequence shown in SEQ ID NO: 1 or at a position corresponding to this position, is deleted or substituted with leucine or valine; and/or
(25) an amino acid residue at position 321 in the amino acid sequence shown in SEQ ID NO: 1 or at a position corresponding to this position, is deleted or substituted with threonine or valine; and/or
(26) an amino acid residue at position 322 in the amino acid sequence shown in SEQ ID NO: 1 or at a position corresponding to this position, is deleted or substituted with threonine; and/or
(27) an amino acid residue at position 327 in the amino acid sequence shown in SEQ ID NO: 1 or at a position corresponding to this position, is deleted or substituted with serine; and/or
(28) an amino acid residue at position 329 in the amino acid sequence shown in SEQ ID NO: 1 or at a position corresponding to this position, is deleted or substituted with serine; and/or
(29) an amino acid residue at position 330 in the amino acid sequence shown in SEQ ID NO: 1 or at a position corresponding to this position, is deleted or substituted with valine; and/or
(30) an amino acid residue at position 331 in the amino acid sequence shown in SEQ ID NO: 1 or at a position corresponding to this position, is deleted or substituted with glycine; and/or
(30) an amino acid residue at position 331 in the amino acid sequence shown in SEQ ID NO: 1 or at a position corresponding to this position, is deleted or substituted with glycine; and/or
(31) an amino acid residue at position 337 in the amino acid sequence shown in SEQ ID NO: 1 or at a position corresponding to this position, is deleted or substituted with threonine; and/or
(32) an amino acid residue at position 338 in the amino acid sequence shown in SEQ ID NO: 1 or at a position corresponding to this position, is deleted or substituted with glycine; and/or
(33) an amino acid residue at position 355 in the amino acid sequence shown in SEQ ID NO: 1 or at a position corresponding to this position, is deleted or substituted with cysteine, threonine or valine; and/or
(34) an amino acid residue at position 365 in the amino acid sequence shown in SEQ ID NO: 1 or at a position corresponding to this position, is deleted or substituted with isoleucine, methionine, serine or threonine; and/or
(35) an amino acid residue at position 378 in the amino acid sequence shown in SEQ ID NO: 1 or at a position corresponding to this position, is deleted or substituted with methionine; and/or
(36) an amino acid residue at position 380 in the amino acid sequence shown in SEQ ID NO: 1 or at a position corresponding to this position, is deleted or substituted with valine; and/or
(37) an amino acid residue at position 384 in the amino acid sequence shown in SEQ ID NO: 1 or at a position corresponding to this position, is deleted or substituted with alanine, glycine, leucine, serine or threonine; and/or
(38) an amino acid residue at position 387 in the amino acid sequence shown in SEQ ID NO: 1 or at a position corresponding to this position, is deleted or substituted with alanine; and/or
(39) an amino acid residue at position 388 in the amino acid sequence shown in SEQ ID NO: 1 or at a position corresponding to this position, is deleted or substituted with methionine, glutamine or serine; and/or
(40) an amino acid residue at position 389 in the amino acid sequence shown in SEQ ID NO: 1 or at a position corresponding to this position, is deleted or substituted with alanine, histidine or glutamine; and/or
(41) an amino acid residue at position 391 in the amino acid sequence shown in SEQ ID NO: 1 or at a position corresponding to this position, is deleted or substituted with isoleucine or leucine; and/or
(42) an amino acid residue at position 392 in the amino acid sequence shown in SEQ ID NO: 1 or at a position corresponding to this position, is deleted or substituted with tyrosine; and/or
(43) an amino acid residue at position 393 in the amino acid sequence shown in SEQ ID NO: 1 or at a position corresponding to this position, is deleted or substituted with alanine; and/or
(44) an amino acid residue at position 394 in the amino acid sequence shown in SEQ ID NO: 1 or at a position corresponding to this position, is deleted or substituted with alanine, cysteine, glycine, histidine, leucine, glutamine or arginine; and/or
(45) an amino acid residue at position 395 in the amino acid sequence shown in SEQ ID NO: 1 or at a position corresponding to this position, is deleted or substituted with valine; and/or
(46) an amino acid residue at position 419 in the amino acid sequence shown in SEQ ID NO: 1 or at a position corresponding to this position, is deleted or substituted with alanine or proline; and/or
(47) at position 424 in the amino acid sequence shown in SEQ ID NO:1 or at a position corresponding to this position, an alanine, aspartate, leucine, serine or tryptophan is inserted; and/or
(48) at position 425 in the amino acid sequence shown in SEQ ID NO:1 or at a position corresponding to this position, a tryptophan is inserted.
(49) an amino acid residue at position 428 in the amino acid sequence shown in SEQ ID NO: 1 or at a position corresponding to this position, is deleted or substituted with methionine or threonine; and/or
(50) an amino acid residue at position 429 in the amino acid sequence shown in SEQ ID NO: 1 or at a position corresponding to this position, is deleted or substituted with alanine, aspartate or valine or a glycine or an asparagine is inserted; and/or
(51) an amino acid residue at position 431 in the amino acid sequence shown in SEQ ID NO: 1 or at a position corresponding to this position, is deleted or substituted with alanine, cysteine, lysine, methionine, asparagine, proline, threonine or valine or a glycine, leucine or asparagine is inserted; and/or
(52) an amino acid residue at position 432 in the amino acid sequence shown in SEQ ID NO: 1 or at a position corresponding to this position, is deleted or substituted with alanine, phenylalanine or tryptophan; and/or
(53) an amino acid residue at position 434 in the amino acid sequence shown in SEQ ID NO: 1 or at a position corresponding to this position, is deleted or substituted with phenylalanine, methionine, valine or tryptophan; and/or
(54) an amino acid residue at position 436 in the amino acid sequence shown in SEQ ID NO: 1 or at a position corresponding to this position, is deleted or substituted with alanine, methionine, asparagine, glutamine, threonine or tryptophan; and/or
(55) an amino acid residue at position 437 in the amino acid sequence shown in SEQ ID NO: 1 or at a position corresponding to this position, is deleted or substituted with phenylalanine, glycine, isoleucine, leucine, methionine, threonine or valine; and/or
(56) an amino acid residue at position 438 in the amino acid sequence shown in SEQ ID NO: 1 or at a position corresponding to this position, is deleted or substituted with leucine, methionine or tryptophan or an aspartate is inserted; and/or
(57) an amino acid residue at position 439 in the amino acid sequence shown in SEQ ID NO: 1 or at a position corresponding to this position, is deleted or substituted with leucine or proline; and/or
(58) an amino acid residue at position 443 in the amino acid sequence shown in SEQ ID NO: 1 or at a position corresponding to this position, is deleted or substituted with histidine or an alanine, glycine, leucine, arginine, threonine or valine is inserted; and/or
(59) an amino acid residue at position 444 in the amino acid sequence shown in SEQ ID NO: 1 or at a position corresponding to this position, is deleted or substituted with phenylalanine, isoleucine, leucine, methionine, asparagine, arginine, threonine or valine or a glutamate, glycine, histidine, lysine, leucine, asparagine, proline, arginine, serine, threonine or valine is inserted; and/or
(60) an amino acid residue at position 446 in the amino acid sequence shown in SEQ ID NO: 1 or at a position corresponding to this position, is deleted or substituted with aspartate, glycine or proline; and/or
(61) an amino acid residue at position 447 in the amino acid sequence shown in SEQ ID NO: 1 or at a position corresponding to this position, is deleted or substituted with phenylalanine, histidine, isoleucine, threonine or tryptophan or a lysine, leucine, proline or tyrosine is inserted; and/or
(62) an amino acid residue at position 448 in the amino acid sequence shown in SEQ ID NO: 1 or at a position corresponding to this position, is deleted or substituted with cysteine, glutamate, threonine or valine or a serine is inserted; and/or
(63) an amino acid residue at position 449 in the amino acid sequence shown in SEQ ID NO: 1 or at a position corresponding to this position, is deleted or substituted with asparagine or tryptophan valine or a serine is inserted; and/or
(64) an amino acid residue at position 451 in the amino acid sequence shown in SEQ ID NO: 1 or at a position corresponding to this position, is deleted or substituted with serine; and/or
(65) an amino acid residue at position 453 in the amino acid sequence shown in SEQ ID NO: 1 or at a position corresponding to this position, is deleted or substituted with leucine; and/or
(66) an amino acid residue at position 457 in the amino acid sequence shown in SEQ ID NO: 1 or at a position corresponding to this position, is deleted or substituted with alanine; and/or
(67) an amino acid residue at position 458 in the amino acid sequence shown in SEQ ID NO: 1 or at a position corresponding to this position, is deleted or substituted with cysteine, glutamate, isoleucine, leucine, methionine, glutamine or arginine or a glycine is inserted; and/or
(68) an amino acid residue at position 459 in the amino acid sequence shown in SEQ ID NO: 1 or at a position corresponding to this position, is deleted or substituted with glycine, leucine, proline, arginine or threonine; and/or
(69) an amino acid residue at position 460 in the amino acid sequence shown in SEQ ID NO: 1 or at a position corresponding to this position, is deleted or substituted glycine, histidine, isoleucine, leucine, methionine, valine or tyrosine; and/or
(70) an amino acid residue at position 462 in the amino acid sequence shown in SEQ ID NO: 1 or at a position corresponding to this position, is deleted or substituted with alanine, cysteine, isoleucine, proline, arginine, threonine or valine or an alanine is inserted; and/or
(71) an amino acid residue at position 464 in the amino acid sequence shown in SEQ ID NO: 1 or at a position corresponding to this position, is deleted or substituted with proline; and/or
(72) an amino acid residue at position 465 in the amino acid sequence shown in SEQ ID NO: 1 or at a position corresponding to this position, is deleted or substituted with alanine, isoleucine or valine; and/or
(73) an amino acid residue at position 467 in the amino acid sequence shown in SEQ ID NO: 1 or at a position corresponding to this position, is deleted or substituted with tryptophan; and/or
(74) an amino acid residue at position 468 in the amino acid sequence shown in SEQ ID NO: 1 or at a position corresponding to this position, is deleted or substituted with leucine; and/or
(75) an amino acid residue at position 470 in the amino acid sequence shown in SEQ ID NO: 1 or at a position corresponding to this position, is deleted or substituted with glycine, serine or threonine; and/or
(76) an amino acid residue at position 471 in the amino acid sequence shown in SEQ ID NO: 1 or at a position corresponding to this position, is deleted or substituted with phenylalanine.

3. The MDC variant of claim 1, wherein said variant furthermore shows at least one modification at positions 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463, 469 in the amino acid sequence shown in SEQ ID NO:1 or at a position corresponding to any of these positions.

4. The MDC variant of claim 1, wherein said variant furthermore shows at least one modification at positions 6, 15, 25, 39, 43, 72, 89, 126, 141, 189, 223, 224, 272, 283, 332, 333, 336, 361, 366, 368, 379, 382, 385, 418, 421, 422, 423, 426, 430, 440, 441, 445, 461, 463, 469 in the amino acid sequence shown in SEQ ID NO:1 or at a position corresponding to any of these positions is an MDC variant, wherein
(1) an amino acid residue at position 6 in the amino acid sequence shown in SEQ ID NO: 1 or at a position corresponding to this position, is deleted or substituted with methionine; and/or
(2) an amino acid residue at position 15 in the amino acid sequence shown in SEQ ID NO: 1 or at a position corresponding to this position, is deleted or substituted with histidine or leucine; and/or
(3) an amino acid residue at position 25 in the amino acid sequence shown in SEQ ID NO: 1 or at a position corresponding to this position, is deleted or substituted with leucine; and/or
(4) an amino acid residue at position 39 in the amino acid sequence shown in SEQ ID NO: 1 or at a position corresponding to this position, is deleted or substituted with glycine; and/or
(5) an amino acid residue at position 43 in the amino acid sequence shown in SEQ ID NO: 1 or at a position corresponding to this position, is deleted or substituted with glutamate; and/or
(6) an amino acid residue at position 72 in the amino acid sequence shown in SEQ ID NO: 1 or at a position corresponding to this position, is deleted or substituted with lysine; and/or
(7) an amino acid residue at position 89 in the amino acid sequence shown in SEQ ID NO: 1 or at a position corresponding to this position, is deleted or substituted with asparagine; and/or
(8) an amino acid residue at position 126 in the amino acid sequence shown in SEQ ID NO: 1 or at a position corresponding to this position, is deleted or substituted with lysine; and/or
(9) an amino acid residue at position 141 in the amino acid sequence shown in SEQ ID NO: 1 or at a position corresponding to this position, is deleted or substituted with cysteine, leucine, glutamine, serine or tyrosine; and/or
(10) an amino acid residue at position 189 in the amino acid sequence shown in SEQ ID NO: 1 or at a position corresponding to this position, is deleted or substituted with cysteine; and/or
(11) an amino acid residue at position 223 in the amino acid sequence shown in SEQ ID NO: 1 or at a position corresponding to this position, is deleted or substituted with tyrosine; and/or
(12) an amino acid residue at position 224 in the amino acid sequence shown in SEQ ID NO: 1 or at a position corresponding to this position, is deleted or substituted with alanine, proline or glutamine; and/or
(13) an amino acid residue at position 272 in the amino acid sequence shown in SEQ ID NO: 1 or at a position corresponding to this position, is deleted or substituted with isoleucine, lysine, leucine or arginine; and/or
(14) an amino acid residue at position 283 in the amino acid sequence shown in SEQ ID NO: 1 or at a position corresponding to this position, is deleted or substituted with cysteine, phenylalanine, histidine, leucine, glutamine, serine, threonine or valine; and/or
(15) an amino acid residue at position 332 in the amino acid sequence shown in SEQ ID NO: 1 or at a position corresponding to this position, is deleted or substituted with histidine, arginine or serine; and/or
(16) an amino acid residue at position 333 in the amino acid sequence shown in SEQ ID NO: 1 or at a position corresponding to this position, is deleted or substituted with methionine; and/or
(17) an amino acid residue at position 336 in the amino acid sequence shown in SEQ ID NO: 1 or at a position corresponding to this position, is deleted or substituted with histidine, asparagine or glutamine; and/or
(18) an amino acid residue at position 361 in the amino acid sequence shown in SEQ ID NO: 1 or at a position corresponding to this position, is deleted or substituted with lysine; and/or
(19) an amino acid residue at position 3665 in the amino acid sequence shown in SEQ ID NO: 1 or at a position corresponding to this position, is deleted or substituted with alanine; and/or
(20) an amino acid residue at position 368 in the amino acid sequence shown in SEQ ID NO: 1 or at a position corresponding to this position, is deleted or substituted with serine; and/or
(21) an amino acid residue at position 379 in the amino acid sequence shown in SEQ ID NO: 1 or at a position corresponding to this position, is deleted or substituted with isoleucine; and/or
(22) an amino acid residue at position 382 in the amino acid sequence shown in SEQ ID NO: 1 or at a position corresponding to this position, is deleted or substituted with alanine or leucine; and/or
(23) an amino acid residue at position 385 in the amino acid sequence shown in SEQ ID NO: 1 or at a position corresponding to this position, is deleted or substituted with aspartate, phenylalanine, glycine, leucine, proline, arginine, serine, threonine or tryptophan; and/or
(24) an amino acid residue at position 418 in the amino acid sequence shown in SEQ ID NO: 1 or at a position corresponding to this position, is deleted or substituted with valine; and/or
(25) an amino acid residue at position 421 in the amino acid sequence shown in SEQ ID NO: 1 or at a position corresponding to this position, is deleted or substituted with phenylalanine, lysine, leucine, threonine, valine or tryptophan or an aspartate, glutamate, glycine, isoleucine, proline, arginine serine or threonine is inserted; and/or
(26) an amino acid residue at position 422 in the amino acid sequence shown in SEQ ID NO: 1 or at a position corresponding to this position, is deleted or substituted with aspartate, phenylalanine, glycine, histidine, leucine, glutamine, serine or valine or a lysine is inserted; and/or
(27) an amino acid residue at position 423 in the amino acid sequence shown in SEQ ID NO: 1 or at a position corresponding to this position, is deleted or substituted with cysteine or glycine; and/or
(28) an amino acid residue at position 426 in the amino acid sequence shown in SEQ ID NO: 1 or at a position corresponding to this position, is deleted or substituted with histidine, isoleucine, leucine, threonine, valine, tryptophan or tyrosine or a leucine is inserted; and/or
(29) an amino acid residue at position 430 in the amino acid sequence shown in SEQ ID NO: 1 or at a position corresponding to this position, is deleted or substituted with alanine, lysine, asparagine, proline, arginine, serine, threonine or valine or a cysteine, glycine, histidine, isoleucine, leucine, asparagine, glutamine, arginine or serine is inserted; and/or
(30) an amino acid residue at position 440 in the amino acid sequence shown in SEQ ID NO: 1 or at a position corresponding to this position, is deleted or substituted with aspartate or isoleucine; and/or
(31) an amino acid residue at position 441 in the amino acid sequence shown in SEQ ID NO: 1 or at a position corresponding to this position, is deleted or substituted with aspartate, glycine, histidine, isoleucine, leucine, proline, threonine or valine; and/or
(32) an amino acid residue at position 445 in the amino acid sequence shown in SEQ ID NO: 1 or at a position corresponding to this position, is deleted or substituted with alanine, glutamine or threonine or a glutamine is inserted; and/or
(33) an amino acid residue at position 461 in the amino acid sequence shown in SEQ ID NO: 1 or at a position corresponding to this position, is deleted or substituted with alanine, aspartate, glutamate, glycine, asparagine, serine, threonine, valine or tyrosine; and/or
(34) an amino acid residue at position 463 in the amino acid sequence shown in SEQ ID NO: 1 or at a position corresponding to this position, is deleted or substituted with glutamate, phenylalanine, glycine, isoleucine, leucine, methionine, proline, serine, threonine or valine; and/or
(35) an amino acid residue at position 469 in the amino acid sequence shown in SEQ ID NO: 1 or at a position corresponding to this position, is deleted or substituted with alanine, aspartate, glutamate, histidine, asparagine, serine or threonine.

5. A nucleic acid molecule encoding the MDC variant of claim 1.

6. A vector comprising the nucleic acid molecule of claim 5.

7. A host cell comprising the nucleic acid molecule of claim 5.

8. A method of converting 3-methylcrotonic acid to isobutene, wherein said method comprises the use of the MDC variant of claim 1 for the conversion of 3-methylcrotonic acid into isobutene.

9. A method for producing isobutene from 3-methylcrotonic acid by incubating 3-methylcrotonic acid with the MDC variant of claim 1.

10. The method of claim 9, wherein the enzymatic conversion is carried out in vitro.

11. A composition comprising a variant of an MDC of claim 1.

12. The composition of claim 11, further comprising 3-methylcrotonic acid.

13. A host cell comprising the vector of claim 7.

14. A method of converting 3-methylcrotonic acid to isobutene, wherein said method comprises the use of the host cell of claim 7 for the conversion of 3-methylcrotonic acid into isobutene.

15. A composition comprising the nucleic acid of claim 5.

16. The composition of claim 15, further comprising 3-methylcrotonic acid.

17. A composition comprising the vector of claim 6.

18. The composition of claim 17, further comprising 3-methylcrotonic acid.

19. A composition comprising the host cell of 7.

20. The composition of claim 19, further comprising 3-methylcrotonic acid.


SEQUENCE LISTING (9 sequences)

SEQ ID NO: 1 - protein, 490 aa, Yersinia frederiksenii
MFLKSLREQINLLKRQGDLRIINRPVDSYLEAAAIIRRSTETKAPVPLMTNIKGYPGCSIVGGLAALSSNPEYPLSRVAMSLGLSTTASAQSIVQYLIDGLKKTPYLPVEIDSNSAPCKQNILRGEDASLAHFPIPQVHQHDGNRYVNTWGVFIVESPDGQWCNWSIQRVQYLNDRQMIALVFPSQHISDIWEEWVKIGKPMPYALVQGCEPIVPYIAGLPLLERNVNEAEYIGALYGKGIEVVKCETHDLRVPASSEVVIEGYFAITREGVEGPFGEFAGYIPNENSLQPILSITAISWQDDPIWPVVAEGKPVDEYHTCSGIGDAAAIQNVLLTANLPVSFVWVPLYSACHLMIVSINRHWRQTHPDLTSEQLTKKVGDVIHQTRNSIKLPKIVVLDDDVDPADTESLLWALATRIHPERKRYFYESCILPLLSCYSEEERHNRKGKRVIIDALLPENHGAISSFDYAYPDDIKQRVLMHWETDFSHD

SEQ ID NO: 2 - protein, 189 aa, Escherichia coli
MKRLIVGISGASGAIYGVRLLQVLRDVTDIETHLVMSQAARQTLSLETDFSLREVQALADVTHDARDIAASISSGSFQTLGMVILPCSIKTLSGIVHSYTDGLLTRAADVVLKERRPLVLCVRETPLHLGHLRLMTQAAEIGAVIMPPVPAFYHRPQSLDDVINQTVNRVLDQFAITLPEDLFARWQGA

SEQ ID NO: 3 - protein, 229 aa, Candida albicans
MIARVCLRRSNVLPIFQIPSRKYSINYEKVNNSIYNNVIKPKRIVLAITGATGTQIGVRLLEILKELGVETHLVMSKWGIATLKYETDYQVDYVTSLATKTYSARDVTAPISSGSFVHDGMIVAPCSMKSLSAIRTGFTEDLIVRAADVSLKERRKLLLVARETPLSDIHLDNMLYLSRMGVTIFPPVPAFYTKPKTIDDIVEQTCGRILDNFGINIDTFERWDGINHR

SEQ ID NO: 4 - protein, 227 aa, Aspergillus niger
MFNSLLSGTTTPNSGRASPPASEMPIDNDHVAVARPAPRRRRIVVAMTGATGAMLGIKVLIALRRLNVETHLVMSKWAEATIKYETDYHPSNVRALADYVHNINDMAAPVSSGSFRADGMIVVPCSMKTLAAIHSGFCDDLISRTADVMLKERRRLVLVARETPLSEIHLRNMLEVTRAGAVIFPPVPAFYIKAGSIEDLIDQSVGRMLDLFDLDTGDFERWNGWEK

SEQ ID NO: 5 - protein, 242 aa, Saccharomyces cerevisiae
MLLFPRRTNIAFFKTTGIFANFPLLGRTITTSPSFLTHKLSKEVTRASTSPPRPKRIVVAITGATGVALGIRLLQVLKELSVETHLVISKWGAATMKYETDWEPHDVAALATKTYSVRDVSACISSGSFQHDGMIVVPCSMKSLAAIRIGFTEDLITRAADVSIKENRKLLLVTRETPLSSIHLENMLSLCRAGVIIFPPVPAFYTRPKSLHDLLEQSVGRILDCFGIHADTFPRWEGIKSK

SEQ ID NO: 6 - protein, 180 aa, Cryptococcus sp.
MRRKRYVVAVTGATGATLAIRLLQALRALDIETHLIISKWAVKTLKYETDMIERELKDLADYSYSNSDLAAPPSSGSFIHDGMFIIPCSMKTLAAVRIGLGDELISRSADVCLKEGRKLMLVVRETPLNDIHLENMLFLRRAGAIIFPPVPAYYIRPQTIDDLTNQTVGRILDSSKCSQK

SEQ ID NO: 7 - protein, 192 aa, Bacillus subtilis
MKLVIGMTGATGAIFGIRLLEYLKAAEIETHLVVSPWANVTITHETDYTLKDVEKLASYTYSHKDQAAAISSGSFETDGMIIAPCSMKSLASIRTGMADNLLTRAADVILKERKKLVLLTRETPLSQIHLENMLALTKMGSVILPPMPAFYNKPADMDELIDHIVFRTLDQFGIRLPEAKRWYGIEKQKGGI

SEQ ID NO: 8 - protein, 209 aa, Pseudomonas aeruginosa
MSGPERITLAMTGASGAQYGLRLLDCLVQEEREVHFLISKAAQLVMATETDVALPAKPQAMQAFLTEYCGAAAGQIRVFGQNDWMAPPASGSSAPNAMVICPCSTGTLSAVATGACNNLIERAADVALKERRPLVLVPREAPFSSIHLENMLKLSNLGAVILPAAPGFYHQPQSVEDLVDFVVARILNTLGIPQDMLPRWGEQHLVSDE

SEQ ID NO: 9 - protein, 219 aa, Enterobacter sp.
MLRQVRANALTCNSPQNPAQSALKSVRAKIMKRLIVGLSGASGAIYGVRLLQVLRNVAEVETHLVMSQAARQTLSLETDLSLRDVQALADVVHDARDIAASISSGSFKTAGMVILPCSIKTLSGIVNSYTDTLVTRAADVVLKERRPLVLCVRETPLHLGHLRLMTQAAELGAIIMPPVPAFYHRPTSLDDVINQTVNRVLDQFDIDLPEDLFTRWQGA